(19) United States
(12) Patent Application Publication
(10) Pub. No.: US 2023/0399370 A1
(43) Pub. Date: Dec. 14, 2023

(54) METHODS OF TREATING GLIOBLASTOMA

(71) Applicant: Alaunos Therapeutics, Inc., Houston, TX (US)

(72) Inventors: Laurence James Neil COOPER, Houston, TX (US); Jill Yanetti BUCK, Andover, MA (US); Arnold Bruce GELB, Cambridge, MA (US); Nathan Aubri DEMARS, North Andover, MA (US); Nira HADAR, Newton, MA (US)

(73) Assignee: Alaunos Therapeutics, Inc., Houston, TX (US)

(21) Appl. No.: 17/778,522

(22) PCT Filed: Nov. 20, 2020
(86) PCT No.: PCT/US2020/061546

§ 371 Date: May 20, 2022

Related U.S. Application Data
(60) Provisional application No. 63/113,018, filed on Nov. 12, 2020.
(60) Provisional application No. 63/025,749, filed on May 15, 2020.
(60) Provisional application No. 62/939,471, filed on Nov. 22, 2019.

(51) Int. Cl. C07K 14/54; C12N 15/86; A61P 35/00

(52) U.S. Cl. CPC C07K 14/5434; C12N 15/86; A61P 35/00; C12N 2830/002; C12N 2710/10343

(57) ABSTRACT

The present invention provides methods of treating glioblastoma by administering an adenoviral vector that conditionally expresses IL-12 via gene switch under control of an oral activator ligand.

### RELATED APPLICATIONS

This application claims benefit of United States Provisional patent application Nos. 62/939,471, filed Nov. 22, 2019, 63/025,749, filed May 15, 2020, and 62/113,018, filed Nov. 12, 2020, the entire content of which is incorporated herein by reference.

### FIELD OF THE INVENTION

The present invention relates generally to controlled IL-12 gene therapy for treatment of glioblastomas.

### BACKGROUND OF THE INVENTION

Interleukin-12 (IL-12) is a member of the type I cytokine family involved in contributing to a number of biological processes including, but not limited to, protective immune response and suppression of tumorigenesis. A growing body of evidence suggests that IL-12 may be a promising target to control cancer.

Despite the fact that IL-12 remains promising as a cancer therapeutic agent based on its potent supportive activity on Type-1 anti-tumor NK cells, CD4⁺ T cells and CD8⁺ T cells, the reported toxicity of recombinant human IL-12 (rhIL-12) in patients, together with limited sources of GMP-grade rhlL-12 for clinical application, have prevented successful IL-12-based therapeutic approaches. Thus, gene therapy approaches represent safer, more tenable treatment options. Indeed, phase I clinical trials implementing intra- or peri-tumoral delivery of recombinant viral- or plasmid-based IL-12 cDNA or IL-12 gene modified autologous fibroblasts have been found to be safe and well-tolerated.

However, objective clinical responses in patients with melanoma or a diverse range of carcinomas receiving these gene therapies have been rare, variable, transient and largely focused at the site of treatment. In cases where disease resolution was partial or complete, increased frequencies of tumor-infiltrating lymphocytes and elevated levels of circulating tumor-specific CD8⁺ T cells have been noted, consistent with the improved cross-priming of antigen-specific T cells in these patients.

Previous use of a recombinant adenovirus encoding mIL-12 under a CMV-based promoter (rAd.cIL12) IL-12 was constitutive, hence the immunologic impact of this cytokine early within the tumor lesion and later within tumor-draining lymph nodes could not be resolved with regard to therapeutic outcome. Thus, a need exists for conditional expression of IL-12 for the purpose of regulating both the level of transgene expression and the timing of the transgene activation.

The present invention solves these needs.

### SUMMARY OF THE INVENTION

The present disclosure provides a method of treating unifocal glioblastoma in a subject in need thereof comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16 transactivation domain-retinoic acid-X-receptor fusion protein (VP-16-RXR) (e.g., the amino acid sequence of SEQ ID NO: 3 or amino acids 2-324 of SEQ ID NO: 3); and (iv) a fourth polynucleotide encoding a Gal4 DNA binding domain and an ecdysone receptor (EcR) binding domain fusion protein (Gal4-EcR) (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby treating the unifocal glioblastoma in the subject.

The present disclosure further provides a method of increasing intratumoral IL-12 expression in a subject having unifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the intratumoral IL-12 expression in the subject.

The present disclosure further provides a method of increasing intratumoral IFN-γ expression in a subject having unifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the intratumoral IFN-γ expression in the subject.

The present disclosure further provides a method of increasing serum IL-12 concentration in a subject having unifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the serum IL-12 concentration in the subject.

The present disclosure further provides a method of increasing serum IFN-γ concentration in a subject having unifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the serum IFN-γ concentration in the subject.

The present disclosure further provides a method of increasing the survival time of a subject having unifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the survival time of the subject.

In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than survival times in subjects not administered the adenoviral vector, e.g., the Ad-RTS-hIL-12 viral vector. In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than the anticipated survival time of a subject according to a control survival time, e.g., a historical control survival time.

In some embodiments, the IL-12 p40 polypeptide is a human IL-12 p40 peptide. In some embodiments, the IL-12 p35 polypeptide is a human IL-12 p35 peptide.

In some embodiments, the first polynucleotide and the second polynucleotide are joined by a first linker. In some embodiments, the third polynucleotide and the fourth polynucleotide are joined by a second linker. In some embodiments, the first linker and/or the second linker is an internal ribosome entry site (IRES) sequence. In some embodiments, the first linker and the second linker are different IRES sequences.

In some embodiments, the vector is a replication-deficient adenoviral vector.

In some embodiments, the subject has not received a steroid for at least 4 weeks prior to injection of the adenoviral vector, e.g., the Ad-RTS-hIL-12 viral vector. In some embodiments, the subject has not previously received bevacizumab.

In some embodiments, an initial dose of the vector and an initial dose of the diacylhydrazine ligand are administered concurrently or sequentially. In some embodiments, an initial dose of the diacylhydrazine ligand is administered at a period of time prior to an initial dose of the vector. In some embodiments, the initial dose of the diacylhydrazine ligand is administered at about 1 to 5 hours prior to the administration of the vector. In some embodiments, one or more subsequent doses of the diacylhydrazine ligand are administered once daily after the administration of the initial dose of the diacylhydrazine ligand. In some embodiments, the subsequent daily doses of the diacylhydrazine ligand are administered for a period of time of about 3-28 days. In some embodiments, the period of time is 14 days.

In some embodiments, the method further comprises administering to the subject a corticosteroid. In some embodiments, the corticosteroid is dexamethasone. In some embodiments, the cumulative dose of corticosteroid during the administration of diacylhydrazine ligand is less than or equal to about 20 mg. In some embodiments, the cumulative dose of dexamethasone during the administration of diacylhydrazine ligand is less than 20 mg at least 14 days after the diacylhydrazine ligand is first administered. In some embodiments, an initial dose of 10 mg of corticosteroid is administered on the same day as the initial dose of the diacylhydrazine ligand. In some embodiments, the corticosteroid is dexamethasone. In some embodiments, the cumulative dose of corticosteroid during the administration of diacylhydrazine ligand is greater than about 20 mg. In some embodiments, the cumulative dose of dexamethasone during the administration of diacylhydrazine ligand is greater than 20 mg at least 14 days after the diacylhydrazine ligand is first administered.

In some embodiments, the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp). In some embodiments, the vector is administered at a dose of about 2×10¹¹ vp.

In some embodiments, the diacylhydrazine ligand is administered at a unit daily dose of about 1 mg to about 120 mg. In some embodiments, the diacylhydrazine ligand is administered at unit daily dose of about 5, 10, 15, 20, 25, 30, 35, 40, 50, 60, 70, 80, 90, 100 or 120 mg.

In some embodiments, the diacylhydrazine ligand is administered at a unit daily dose of about 5 mg. In some embodiments, the diacylhydrazine ligand is administered at a unit daily dose of about 10 mg. In some embodiments, the diacylhydrazine ligand is administered at a unit daily dose of about 15 mg. In some embodiments, the diacylhydrazine ligand is administered at a unit daily dose of about 20 mg. In some embodiments, the diacylhydrazine ligand is administered at a unit daily dose of about 30 mg. In some embodiments, the diacylhydrazine ligand is administered at a unit daily dose of about 40 mg. In some embodiments, the diacylhydrazine ligand is veledimex.

In some embodiments, the method further comprises selecting the subject with unifocal glioblastoma before injecting the adenoviral vector, e.g., the Ad-RTS-hIL-12 viral vector, or administering (e.g., orally administering) the diacylhydrazine ligand. In some embodiments, the subject has been diagnosed with unifocal glioblastoma.

The present disclosure further provides a method of treating unifocal glioblastoma in a subject in need thereof comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby treating the unifocal glioblastoma in the subject.

The present disclosure further provides a method of increasing intratumoral IL-12 expression in a subject having unifocal glioblastoma comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the intratumoral IL-12 expression in the subject.

The present disclosure further provides a method of increasing intratumoral IFN-γ expression in a subject having unifocal glioblastoma comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the intratumoral IFN-γ expression in the subject.

The present disclosure further provides a method of increasing serum IL-12 concentration in a subject having unifocal glioblastoma comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the serum IL-12 concentration in the subject.

The present disclosure further provides a method of increasing serum IFN-γ concentration in a subject having unifocal glioblastoma comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the serum IFN-γ concentration in the subject.

The present disclosure further provides a method of increasing the survival time of a subject having unifocal glioblastoma comprising (a) selecting a subject with unifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), wherein the subject is also administered dexamethasone at a cumulative dose of less than 20 mg for at least two weeks after veledimex is first administered, thereby increasing the survival time of the subject.

In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than survival times in subjects not administered the adenoviral vector, e.g., the Ad-RTS-hIL-12 viral vector. In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than the anticipated survival time of a subject according to a control survival time, e.g., a historical control survival time.

In some embodiments, the IL-12 p40 polypeptide is a human IL-12 p40 peptide. In some embodiments, the IL-12 p35 polypeptide is a human IL-12 p35 peptide.

In some embodiments, the first polynucleotide and the second polynucleotide is joined by a first linker. In some embodiments, the third polynucleotide and the fourth polynucleotide is joined by a second linker. In some embodiments, the first linker and/or the second linker is an internal ribosome entry site (IRES) sequence. In some embodiments, the first linker and the second linker are different IRES sequences.

In some embodiments, the vector is a replication-deficient adenoviral vector.

In some embodiments, the subject has not received a steroid for at least 4 weeks prior to injection of the adenoviral vector, e.g., the Ad-RTS-hIL-12 viral vector. In some embodiments, the subject has not previously received bevacizumab.

In some embodiments, an initial dose of the vector and an initial dose of veledimex are administered concurrently or sequentially. In some embodiments, an initial dose of veledimex is administered at a period of time prior to an initial dose of the vector. In some embodiments, the initial dose of veledimex is administered at about 1 to 5 hours prior to the administration of the vector. In some embodiments, one or more subsequent doses of veledimex are administered once daily after the administration of the initial dose of veledimex. In some embodiments, the subsequent daily doses of veledimex are administered for a period of time of about 3-28 days. In some embodiments, the period of time is 14 days.

In some embodiments, the method further comprises administering to the subject a corticosteroid. In some embodiments, the corticosteroid is dexamethasone. In some embodiments, the cumulative dose of corticosteroid during the administration of veledimex is less than or equal to about 20 mg. In some embodiments, the cumulative dose of dexamethasone during the administration of veledimex is less than 20 mg at least 14 days after the veledimex is first administered. In some embodiments, an initial dose of 10 mg of corticosteroid is administered on the same day as the initial dose of veledimex. In some embodiments, the corticosteroid is dexamethasone. In some embodiments, the cumulative dose of corticosteroid during the administration of veledimex is greater than about 20 mg. In some embodiments, the cumulative dose of dexamethasone during the administration of veledimex is greater than 20 mg at least 14 days after the veledimex is first administered.

In some embodiments, the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp). In some embodiments, the vector is administered at a dose of about 2×10¹¹ vp.

In some embodiments, the veledimex is administered at a unit daily dose of about 1 mg to about 120 mg. In some embodiments, the veledimex is administered at unit daily dose of about 5, 10, 15, 20, 25, 30, 35, 40, 50, 60, 70, 80, 90, 100 or 120 mg.

In some embodiments, the veledimex is administered at a unit daily dose of about 5 mg. In some embodiments, the veledimex is administered at a unit daily dose of about 10 mg. In some embodiments, the veledimex is administered at a unit daily dose of about 15 mg. In some embodiments, the veledimex is administered at a unit daily dose of about 20 mg.

The present disclosure further provides a method of treating multifocal glioblastoma in a subject in need thereof comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16 transactivation domain-retinoic acid-X-receptor fusion protein (VP-16-RXR) (e.g., the amino acid sequence of SEQ ID NO: 3 or amino acids 2-324 of SEQ ID NO: 3); and (iv) a fourth polynucleotide encoding a Gal4 DNA binding domain and an ecdysone receptor (EcR) binding domain fusion protein (Gal4-EcR) (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby treating the multifocal glioblastoma in the subject.

The present disclosure further provides a method of increasing intratumoral IL-12 expression in a subject having multifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the intratumoral IL-12 expression in the subject.

The present disclosure further provides a method of increasing intratumoral IFN-γ expression in a subject having multifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the intratumoral IFN-γ expression in the subject.

The present disclosure further provides a method of increasing serum IL-12 concentration in a subject having multifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the serum IL-12 concentration in the subject.

The present disclosure further provides a method of increasing serum IFN-γ concentration in a subject having multifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the serum IFN-7 concentration in the subject.

The present disclosure further provides a method of increasing the survival time of a subject having multifocal glioblastoma comprising (a) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (b) administering (e.g., orally administering) to the subject a diacylhydrazine ligand, e.g., veledimex (e.g., a therapeutically effective amount of the diacylhydrazine ligand, e.g., veledimex) that activates the ligand-dependent transcription factor complex, thereby increasing the survival time of the subject.

In some embodiments, the IL-12 p40 polypeptide is a human IL-12 p40 peptide. In some embodiments, the IL-12 p35 polypeptide is a human IL-12 p35 peptide.

In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than survival times in subjects not administered the adenoviral vector, e.g., the Ad-RTS-hIL-12 viral vector. In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than the anticipated survival time of a subject according to a control survival time, e.g., a historical control survival time.

In some embodiments, the first polynucleotide and the second polynucleotide are joined by a first linker. In some embodiments, the third polynucleotide and the fourth polynucleotide are joined by a second linker. In some embodiments, the first linker and/or the second linker is an internal ribosome entry site (IRES) sequence. In some embodiments, the first linker and the second linker are different IRES sequences.

In some embodiments, the vector is a replication-deficient adenoviral vector.

In some embodiments, the subject has not received a steroid for at least 4 weeks prior to injection of the adenoviral vector, e.g., the Ad-RTS-hIL-12 viral vector. In some embodiments, the subject has not previously received bevacizumab.

In some embodiments, an initial dose of the vector and an initial dose of the diacylhydrazine ligand are administered concurrently or sequentially. In some embodiments, an initial dose of the diacylhydrazine ligand is administered at a period of time prior to an initial dose of the vector. In some embodiments, the initial dose of the diacylhydrazine ligand is administered at about 1 to 5 hours prior to the administration of the vector. In some embodiments, one or more subsequent doses of the diacylhydrazine ligand are administered once daily after the administration of the initial dose of the diacylhydrazine ligand. In some embodiments, the subsequent daily doses of the diacylhydrazine ligand are administered for a period of time of about 3-28 days. In some embodiments, the period of time is 14 days.

In some embodiments, the method further comprises administering to the subject a corticosteroid. In some embodiments, the corticosteroid is dexamethasone. In some embodiments, the cumulative dose of corticosteroid during the administration of diacylhydrazine ligand is less than or equal to about 20 mg. In some embodiments, the cumulative dose of dexamethasone during the administration of diacylhydrazine ligand is less than 20 mg at least 14 days after the diacylhydrazine ligand is first administered. In some embodiments, an initial dose of 10 mg of corticosteroid is administered on the same day as the initial dose of the diacylhydrazine ligand. In some embodiments, the corticosteroid is dexamethasone. In some embodiments, the cumulative dose of corticosteroid during the administration of diacylhydrazine ligand is greater than about 20 mg. In some embodiments, the cumulative dose of dexamethasone during the administration of diacylhydrazine ligand is greater than 20 mg at least 14 days after the diacylhydrazine ligand is first administered.

In some embodiments, the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp). In some embodiments, the vector is administered at a dose of about 2×10¹¹ vp.

In some embodiments, the diacylhydrazine ligand is administered at a unit daily dose of about 1 mg to about 120 mg. In some embodiments, the diacylhydrazine ligand is administered at unit daily dose of about 5, 10, 15, 20, 25, 30, 35, 40, 50, 60, 70, 80, 90, 100 or 120 mg.

In some embodiments, the diacylhydrazine ligand is administered at a unit daily dose of about 5 mg. In some embodiments, the diacylhydrazine ligand is administered at a unit daily dose of about 10 mg. In some embodiments, the diacylhydrazine ligand is administered at a unit daily dose of about 15 mg. In some embodiments, the diacylhydrazine ligand is administered at a unit daily dose of about 20 mg. In some embodiments, the diacylhydrazine ligand is administered at a unit daily dose of about 30 mg. In some embodiments, the diacylhydrazine ligand is administered at a unit daily dose of about 40 mg. In some embodiments, the diacylhydrazine ligand is veledimex.

In some embodiments, the method further comprises selecting the subject with multifocal glioblastoma before injecting the adenoviral vector, e.g., the Ad-RTS-hIL-12 viral vector, or administering (e.g., orally administering) the diacylhydrazine ligand. In some embodiments, the subject has been diagnosed with multifocal glioblastoma

The present disclosure further provides a method of treating multifocal glioblastoma in a subject in need thereof comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby treating the multifocal glioblastoma in the subject.

The present disclosure further provides a method of increasing intratumoral IL-12 expression in a subject having multifocal glioblastoma comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the intratumoral IL-12 expression in the subject.

The present disclosure further provides a method of increasing intratumoral IFN-γ expression in a subject having multifocal glioblastoma comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the intratumoral IFN-γ expression in the subject.

The present disclosure further provides a method of increasing serum IL-12 concentration in a subject having multifocal glioblastoma comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the serum IL-12 concentration in the subject.

The present disclosure further provides a method of increasing serum IFN-γ concentration in a subject having multifocal glioblastoma comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), thereby increasing the serum IFN-γ concentration in the subject.

The present disclosure further provides a method of increasing the survival time of a subject having multifocal glioblastoma comprising (a) selecting a subject with multifocal glioblastoma; (b) intratumorally injecting (e.g., injection into an existing tumor or the periphery thereof or into a tumor resection site or the periphery thereof; e.g., injection intraoperatively into the cavity wall immediately following tumor resection; e.g., injecting into the glioblastoma or periphery thereof or into a resection site of the glioblastoma or periphery thereof, e.g., injecting intraoperatively into the cavity wall immediately following glioblastoma resection) into the subject an adenoviral vector, e.g., an Ad-RTS-hIL-12 viral vector (e.g., a therapeutically effective amount of the adenoviral vector, e.g., the Ad-RTS-hIL-12 viral vector), wherein the vector comprises: (i) a first polynucleotide encoding an IL-12 p40 polypeptide comprising an amino acid sequence at least 85% identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence at least 85% identical to wild-type human IL-12 p35 polypeptide (e.g., the amino acid sequence of SEQ ID NO: 2 or amino acids 23-219 of SEQ ID NO: 2); (iii) a third polynucleotide encoding VP-16-RXR (e.g., the amino acid sequence of SEQ ID NO: 3 or amino acids 2-324 of SEQ ID NO: 3); and (iv) a fourth polynucleotide encoding Gal4-EcR (e.g., the amino acid sequence of SEQ ID NO: 4 or amino acids 2-488 of SEQ ID NO: 4), wherein the VP-16-RXR fusion protein and the Gal4-EcR fusion protein form a ligand dependent transcription factor complex; and (c) administering (e.g., orally administering) to the subject daily veledimex (e.g., a therapeutically effective amount of veledimex), wherein the subject is also administered dexamethasone at a cumulative dose of less than 20 mg for at least two weeks after veledimex is first administered, thereby increasing the survival time of the subject.

In some embodiments, the IL-12 p40 polypeptide is a human IL-12 p40 peptide. In some embodiments, the IL-12 p35 polypeptide is a human IL-12 p35 peptide.

In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than survival times in subjects not administered the adenoviral vector, e.g., the Ad-RTS-hIL-12 viral vector. In some embodiments, the increase in survival time is at least 1.1-fold, 1.2-fold, 1.3-fold, 1.4-fold, 1.5-fold, 1.6-fold, 1.7-fold, 1.8-fold, 1.9-fold, or 2.0-fold higher than the anticipated survival time of a subject according to a control survival time, e.g., a historical control survival time.

In some embodiments, the first polynucleotide and the second polynucleotide is joined by a first linker. In some embodiments, the third polynucleotide and the fourth polynucleotide is joined by a second linker. In some embodiments, the first linker and/or the second linker is an internal ribosome entry site (IRES) sequence. In some embodiments, the first linker and the second linker are different IRES sequences.

In some embodiments, the vector is a replication-deficient adenoviral vector.

In some embodiments, the subject has not received a steroid for at least 4 weeks prior to injection of the adenoviral vector, e.g., the Ad-RTS-hIL-12 viral vector. In some embodiments, the subject has not previously received bevacizumab.

In some embodiments, an initial dose of the vector and an initial dose of veledimex are administered concurrently or sequentially. In some embodiments, an initial dose of veledimex is administered at a period of time prior to an initial dose of the vector. In some embodiments, the initial dose of veledimex is administered at about 1 to 5 hours prior to the administration of the vector. In some embodiments, one or more subsequent doses of veledimex are administered once daily after the administration of the initial dose of veledimex. In some embodiments, the subsequent daily doses of veledimex are administered for a period of time of about 3-28 days. In some embodiments, the period of time is 14 days.

In some embodiments, the method further comprises administering to the subject a corticosteroid. In some embodiments, the corticosteroid is dexamethasone. In some embodiments, the cumulative dose of corticosteroid during the administration of veledimex is less than or equal to about 20 mg. In some embodiments, an initial dose of 10 mg of corticosteroid is administered on the same day as the initial dose of veledimex. In some embodiments, the corticosteroid is dexamethasone. In some embodiments, the cumulative dose of corticosteroid during the administration of veledimex is greater than about 20 mg. In some embodiments, the cumulative dose of dexamethasone during the administration of veledimex is greater than 20 mg at least 14 days after veledimex is first administered.

In some embodiments, the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp). In some embodiments, the vector is administered at a dose of about 2×10¹¹ vp.

In some embodiments, veledimex is administered at a unit daily dose of about 1 mg to about 120 mg. In some embodiments, veledimex is administered at unit daily dose of about 5, 10, 15, 20, 25, 30, 35, 40, 50, 60, 70, 80, 90, 100 or 120 mg.

In some embodiments, veledimex is administered at a unit daily dose of about 5 mg. In some embodiments, veledimex is administered at a unit daily dose of about 10 mg. In some embodiments, veledimex is administered at a unit daily dose of about 15 mg. In some embodiments, veledimex is administered at a unit daily dose of about 20 mg.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents and other references mentioned herein are expressly incorporated by reference in their entirety. In cases of conflict, the present specification, including definitions, will control. In addition, the materials, methods and examples described herein are illustrative only and are not intended to be limiting.

Other features and advantages of the invention will be apparent from and encompassed by the following detailed description and claims.

### BRIEF DESCRIPTION OF THE DRAWINGS

FIGS. 1A and 1B are schematic representations of vectors. FIG. 1A depicts the structure of an Ad-RTS-hIL-12 viral vector in which part or all of the E1 and E3 regions have been deleted and gene switch components (sometimes designated “RTS” for “RHEOSWITCH THERAPEUTIC SYSTEM”) replace the E1 region. The box labeled “IL 12” represents the IL-12p40 and IL-12p35 coding sequences separated by an IRES (Internal Ribosome Entry Site) polynucleotide sequence. FIG. 1B depicts the structure of an alternative hIL-12 vector incorporating an RTS switch.

FIGS. 2A and 2B are schematic representations of dosing regimens for Ad-RTS-hIL-12 plus veledimex therapy. FIG. 2A describes the “Main Study.” FIG. 2B describes the “Expansion Substudy” (“Substudy”).

FIG. 3 shows a schematic diagram illustrating how Ad-RTS-hIL-12+veledimex therapy drives downstream production of IFN-7 and other cytokines via a cascade that elicits a brisk cytotoxic immune response.

FIGS. 4A-4C are graphs showing sustained serum cytokine production during dosing and immune activation in subjects from the Main Study and the Substudy. FIG. 4A shows the serum IL-12 production on Days 0, 3, 7, 14, and 28. FIG. 4B shows serum Interferon-gamma (IFN-γ) production on Days 0, 3, 7, 14, and 28. Together, this shows sustained intratumoral production of cytokines in serum from recurrent glioblastoma patients. FIG. 4C shows “cytoindex” ratios of cytotoxic Tcells/Tregs as measured by the CD3⁺CD8⁺/CD3⁺CD4⁺CD25^{hi}FOXP3⁺CD127^{lo/-} immune cell ratio using flow cytometry. IL-12 mediated activation of peripheral blood immune cells was assessed by serial measurement of the cytoindex on Days 0, 7, 14, and 28. An increase in cytoindex indicates an enhanced peripheral cytotoxic immune response.

FIGS. 5A and 5B depicts graphs showing sustained serum cytokine production during dosing and immune activation in subjects from the Main Study and the Substudy. FIG. 5A shows serum IL-12 production on Days 0, 3, 7, 14, and 28. FIG. 5B shows interferon gamma (IFN-γ) production on Days 0, 3, 7, 14, and 28. The data are segregated between patients administered ≤20 mg dexamethasone vs. >20 mg dexamethasone during veledimex dosing.

FIG. 6 depicts serial MRI images showing evidence of immune mediated anti-tumor response. Five MRI scanned images of a 51-year-old female from the Expansion substudy are depicted (Pre-Baseline, Baseline, 28 Days after Ad+V, 56 Days after Ad+V; 40 Weeks after Ad+V). Images show pseudoprogression at Day 28 with the lesion improving and becoming non-measurable by Day 56 continuing through Week 40 (partial response per iRANO criteria).

FIG. 7 shows a survival curve of unifocal vs multifocal subjects from the Main Study and the Expansion Substudy (independent of steroids).

FIG. 8 shows a survival curve of unifocal subjects from the Main Study and the Expansion Substudy depicted by cumulative concurrent steroid dosage during veledimex dosing (≤20 mg dexamethasone vs. >20 mg dexamethasone).

FIGS. 9A-9C show veledimex concentrations in the cerebrospinal fluid (CSF), tumor samples and plasma of patients in the Main Study. FIG. 9A shows CSF veledimex concentrations in patients receiving 20 mg or 30 mg veledimex. FIG. 9B shows tumor veledimex concentrations in patients receiving 10 mg, 20 mg, 30 mg or 40 mg veledimex in the resection group. FIG. 9C shows plasma concentrations of veledimex in patients receiving 10 mg, 20 mg, 30 mg or 40 mg veledimex in Group 1 as well as patients receiving 20 mg veledimex in Group 2.

FIG. 10 depicts serial MRI images showing progressive disease on imaging at Day 14 following Ad+V was subsequently shown to be pseudoprogression (PsP) with stable disease at Week 16 and 40, followed by partial response at Week 72 and Week 96. MRI scan images of a 51-year-old male from the Main Study are depicted.

FIG. 11 depicts serial MRI images showing progressive disease on imaging at Day 28 following Ad+V was subsequently shown to be pseudoprogression (PsP) with stable disease at Day 56 and partial response based on scans at Week 30 and Week 48. MRI scan images of a 40 year old male with one prior line of therapy are depicted.

FIG. 12 shows a survival curve of subjects from the Main Study, separated by cohorts from Group 1 (10 mg, 30 mg or 40 mg veledimex and 20 mg of veledimex) and Group 2.

FIG. 13 shows a survival curve of unifocal subjects from Group 1 of the Main Study, comparing the combined 10 mg, 30 mg and 40 mg cohorts and the 20 mg cohort from Group 1 with ≤20 mg dexamethasone vs. >20 mg dexamethasone during veledimex dosing.

### DETAILED DESCRIPTION OF THE INVENTION

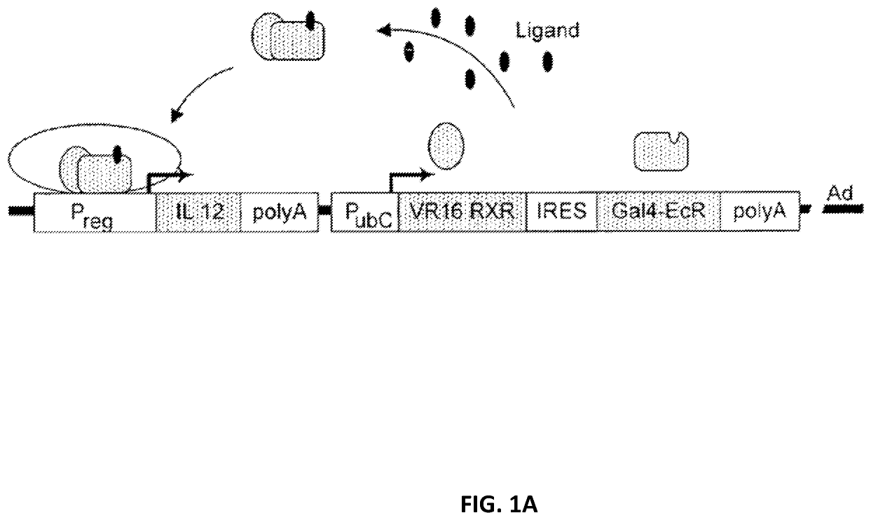

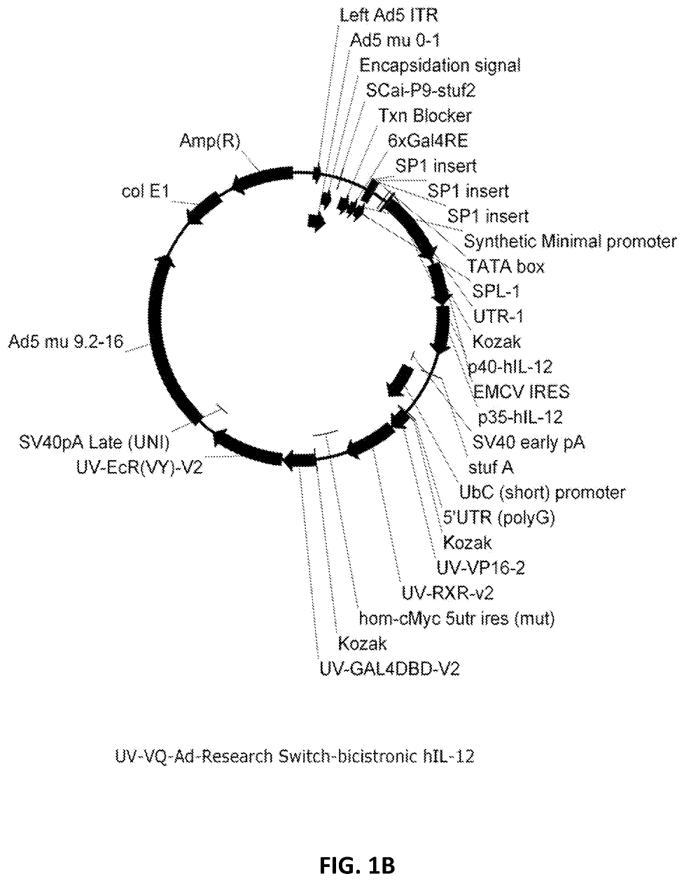

The invention is based in part upon the discovery that administration of Ad-RTS-hIL-12, and optionally low-dose dexamethasone, to patients with recurrent glioblastoma surprisingly resulted in an increase in survival for patients having unifocal disease. Ad-RTS-hIL-12 refers to an adenoviral polynucleotide vector harboring the human IL-12 p40 and p35 genes (hIL-12) under the control of a gene switch of the RheoSwitch Therapeutic System® (RTS®), which can express the IL-12 protein in the presence of activating ligand, e.g., as shown schematically in FIG. 1A. In particular, an adenoviral vector (e.g. the Ad-RTS-hIL-12 viral vector) encodes (i) a first polynucleotide encoding an IL-12 p40 polypeptide comprising an amino acid sequence, e.g., at least 75%, 80%, 85%, 90%, 91%, 92%, 93%, 94%, 95%, 96%, 97%, 98%, 99%, 100%, or any percentage in between, identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence, e.g., at least 75%, 80%, 85%, 90%, 91%, 92%, 93%, 94%, 95%, 96%, 97%, 98%, 99%, 100%, or any percentage in between, identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16 transactivation domain-retinoic acid-X-receptor fusion protein (VP-16-RXR), e.g., comprising an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 3 to or amino acids 2-324 of SEQ ID NO: 3; and (iv) a fourth polynucleotide encoding a Gal4 DNA binding domain and an ecdysone receptor (EcR) binding domain fusion protein (Gal4-EcR), e.g., comprising an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 4 or to amino acids 2-488 of SEQ ID NO: 4, wherein the VP-16-RXR fusion protein and the Gal4-EcR fusion protein form a ligand dependent transcription factor complex. The activating ligand is a diacylhydrazine ligand such as veledimex.

Thus, in various embodiments the invention provides methods of delaying the progression of, treating, increasing survival time for, alleviating a symptom of or otherwise ameliorating or treating one or more symptoms of glioblastoma in a subject, or increasing intratumoral expression of IL-12 in a subject, by administering a therapeutically effective amount of an adenoviral vector, e.g., an Ad-RTS-hIL-12 viral vector, and an activating ligand. By increasing survival time of a subject, it is meant that the subject survives for a period of time of at least 1.1, 1.2, 1.3, 1.4, 1.5, 1.6, 1.7, 1.8, 1.9, 2.0-fold or more than a typical subject left untreated or treated with conventional therapies for glioblastoma.

Glioblastoma, also known as glioblastoma multiforme (GBM), is one of the most aggressive cancers that begins within the brain. Initially, signs and symptoms of glioblastoma are nonspecific. These may include headaches, personality changes, nausea and symptoms similar to those of a stroke. The diagnosis typically is made by a combination of clinical findings from CT scan, MRI scan and tissue biopsy. Despite aggressive treatment, the cancer usually recurs. The typical length of survival following diagnosis is 12 to 15 months, with fewer than of 3 to 7% people surviving longer than five years. Without any type of treatment, survival is typically three months. The glioblastoma may be primary glioblastoma, secondary glioblastoma, recurrent glioblastoma or progressive glioblastoma. The glioblastoma may be unifocal glioblastoma or multifocal glioblastoma.

### Adenoviral Vectors for Gene Delivery

Suitable viral vectors used in the present disclosure include, but are not limited to, adenovirus-based vectors. Adenovirus (Ad) is a 36 kb double-stranded DNA virus that efficiently transfers DNA in vivo to a variety of different target cell types. The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. In certain embodiments, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, WO 00/00628, and WO 2020/006274, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996), incorporated herein by reference.

In other embodiments, the adenoviral vector is replication-deficient. The term “replication-deficient” used herein means that the adenoviral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient adenoviral vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). In other embodiments, the replication-deficient adenoviral vector comprises an adenoviral genome deficient in at least one replication-essential gene function of one or more regions of an adenoviral genome (e.g., two or more regions of an adenoviral genome to result in a multiply replication-deficient adenoviral vector). The one or more regions of the adenoviral genome are selected from the group consisting of the E1, E2, and E4 regions. The can comprise a deficiency in at least one replication-essential gene function of the E1 region (denoted an E1-deficient adenoviral vector), particularly a deficiency in a replication-essential gene function of each of the adenoviral E1A region and the adenoviral E1B region. In addition to such a deficiency in the E1 region, the recombinant adenovirus also can have a mutation in the major late promoter (MLP), as discussed in International Patent Application WO 00/000628, incorporated by reference, herein. In a particular embodiment, the vector is deficient in at least one replication-essential gene function of the E1 region and at least part of the nonessential E3 region (e.g., an Xba I deletion of the E3 region) (denoted an E1/E3-deficient adenoviral vector).

In certain embodiments, the adenoviral vector is “multiply-deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Alternatively, the adenoviral vector lacks replication-essential gene functions in all or part of the E1 region and all or part of the E2 region (denoted an E1/E2-deficient adenoviral vector). Adenoviral vectors lacking replication-essential gene functions in all or part of the E1 region, all or part of the E2 region, and all or part of the E3 region are also contemplated herein. If the adenoviral vector of the invention is deficient in a replication-essential gene function of the E2A region, the vector does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993), incorporated by reference, herein). While deletions in the E2A region coding for the Ct region of the DBP have no effect on viral replication, deletions in the E2A region which code for amino acids 2 to 38 of the Nt domain of the DBP impair viral replication. In one embodiment, the multiply replication-deficient adenoviral vector contains this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient adenoviral vectors, including multiply deficient adenoviral vectors, are disclosed in U.S. Pat. Nos. 5,851,806 and 5,994,106 and International Patent Applications WO 95/34671 and WO 97/21826, incorporated by reference, herein. In one embodiment, the vector for use in the present disclosure is that described in International Patent Application PCT/US01/20536, incorporated by reference, herein.

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified to decrease the adenoviral vector's ability or inability to be recognized by a neutralizing antibody directed against the wild-type coat protein, as described in International Patent Application WO 98/40509, incorporated by reference, herein.

The adenoviral vector, when multiply replication-deficient, especially in replication-essential gene functions of the E1 and E4 regions, can include a spacer element to provide viral growth in a complementing cell line similar to that achieved by singly replication deficient adenoviral vectors, particularly an adenoviral vector comprising a deficiency in the E1 region. The spacer element can contain any sequence or sequences which are of the desired length. The spacer element sequence can be coding or non-coding and native or non-native with respect to the adenoviral genome, but it does not restore the replication-essential function to the deficient region. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. The use of a spacer in an adenoviral vector is described in U.S. Pat. No. 5,851,806. Construction of adenoviral vectors is well understood in the art. Adenoviral vectors can be constructed and/or purified using the methods set forth, for example, in U.S. Pat. No. 5,965,358 and International Patent Applications WO 98/56937, WO 99/15686, and WO 99/54441, incorporated by reference, herein. The production of adenoviral gene transfer vectors is well known in the art, and involves using standard molecular biological techniques such as those described in, for example, Sambrook el al. Molecular Cloning: A Laboratory Manual (2d ed., Cold Spring Harbor Laboratory Press, Cold Spring Harbor, N.Y. (1989)), Watson J D, Gilman M, Witkowski J & Zoller M (1992) Recombinant DNA, 2nd edn. New York: W H Freeman and Ausubel et al, Short Protocols in Molecular Biology, John Wiley and Sons, New York, N.Y. (1992) and in several of the other references mentioned herein.

Replication-deficient adenoviral vectors are typically produced in complementing cell lines that provide gene functions not present in the replication-deficient adenoviral vectors, but required for viral propagation, at appropriate levels in order to generate high titers of viral vector stock. In one embodiment, a cell line complements for at least one and/or all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only inverted terminal repeats (ITRs) and the packaging signal or only ITRs and an adenoviral promoter). In another embodiment, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. The construction of complementing cell lines involves standard molecular biology and cell culture techniques, such as those described by Sambrook et al., supra, and Ausubel et al., supra. Complementing cell lines for producing the gene transfer vector (e.g., adenoviral vector) include, but are not limited to, 293 cells (described in, e.g., Graham et al., J. Gen. Virol., 36, 59-72 1977, incorporated by reference, herein), PER.C6 cells (described in, e.g., International Patent Application WO 97/00326, and U.S. Pat. Nos. 5,994,128 and 6,033,908, incorporated by reference, herein), and 293-ORF6 cells (described in, e.g., International Patent Application WO 95/34671 and Brough et al., J Virol., 71, 9206-9213 1997, incorporated by reference, herein). The insertion of a nucleic acid sequence into the adenoviral genome (e.g., the E1 region of the adenoviral genome) can be facilitated by known methods, for example, by the introduction of a unique restriction site at a given position of the adenoviral genome.

The polynucleotide sequence in the expression vector is operatively linked to appropriate expression control sequence(s) including, for instance, a promoter to direct mRNA transcription. Representatives of additional promoters include, but are not limited to, constitutive promoters and tissue specific or inducible promoters. Examples of constitutive eukaryotic promoters include, but are not limited to, the promoter of the mouse metallothionein I gene (Hamer et al., J. Mol. Appl. Gen. 1:273 1982, incorporated by reference, herein); the TK promoter of Herpes virus (McKnight, Cell 31:355 1982, incorporated by reference, herein); the SV40 early promoter (Benoist et al., Nature 290:304 1981, incorporated by reference, herein); and the vaccinia virus promoter. Additional examples of the promoters that can be used to drive expression of a protein or polynucleotide include, but are not limited to, tissue-specific promoters and other endogenous promoters for specific proteins, such as the albumin promoter (hepatocytes), a proinsulin promoter (pancreatic beta cells) and the like. In general, expression constructs will contain sites for transcription, initiation and termination and, in the transcribed region, a ribosome binding site for translation. The coding portion of the mature transcripts expressed by the constructs may include a translation initiating AUG at the beginning and a termination codon (UAA, UGA or UAG) appropriately positioned at the end of the polypeptide to be translated.

### Gene Switch Systems

The gene switch utilized in the disclosure herein may be any gene switch that regulates gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an ecdysone receptor (EcR)-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric EcR systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, WO 2005/108617, and WO 2020/006274, each of which is incorporated by reference in its entirety.

In another aspect of the invention, the gene switch is based on heterodimerization of FK506 binding protein (FKBP) with FKBP rapamycin associated protein (FRAP) and is regulated through rapamycin or its non-immunosuppressive analogs. Examples of such systems include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595.

In one embodiment, the gene switch comprises a single transcription factor sequence encoding a ligand-dependent transcription factor complex under the control of a therapeutic switch promoter. The transcription factor sequence may encode a ligand-dependent transcription factor complex that is a naturally occurring or an artificial An artificial transcription factor is one in which the natural sequence of the transcription factor has been altered, e.g., by mutation of the sequence or by the combining of domains from different transcription factors. In one embodiment, the transcription factor comprises a Group H nuclear receptor ligand binding domain. In one embodiment, the Group H nuclear receptor ligand binding domain is from an EcR, a ubiquitous receptor (UR), an orphan receptor 1 (OR-1), a steroid hormone nuclear receptor 1 (NER-1), a retinoid X receptor interacting protein-15 (RIP-15), a liver X receptor β (LXRβ), a steroid hormone receptor like protein (RLD-1), a liver X receptor (LXR), a liver X receptor α (LXRα), a farnesoid X receptor (FXR), a receptor interacting protein 14 (RIP-14), or a farnesol receptor (HRR-1). In another embodiment, the Group H nuclear receptor LBD is from an EcR.

The EcR and the other Group H nuclear receptors are members of the nuclear receptor superfamily wherein all members are generally characterized by the presence of an amino-terminal transactivation domain (AD, also referred to interchangeably as “TA” or “TD”), optionally fused to a heterodimerization partner (HP) to form a coactivation protein (CAP), a DNA binding domain (DBD), and an ligand binding domain (LBD) fused to the DBD via a hinge region to form a (LTF). As used herein, the term “DNA binding domain” comprises a minimal polypeptide sequence of a DNA binding protein, up to the entire length of a DNA binding protein, so long as the DNA binding domain functions to associate with a particular response element. Members of the nuclear receptor superfamily are also characterized by the presence of four or five domains: A/B, C, D, E, and in some members F (see U.S. Pat. No. 4,981,784 and Evans, Science 240:889 (1988)). The “A/B” domain corresponds to the transactivation domain, “C” corresponds to the DNA binding domain, “D” corresponds to the hinge region, and “E” corresponds to the ligand binding domain. Some members of the family may also have another transactivation domain on the carboxy-terminal side of the LBD corresponding to “F”.

The DBD is characterized by the presence of two cysteine zinc fingers between which are two amino acid motifs, the P-box and the D-box, which confer specificity for response elements. These domains may be either native, modified, or chimeras of different domains of heterologous receptor proteins. The EcR, like a subset of the nuclear receptor family, also possesses less well-defined regions responsible for heterodimerization properties. Because the domains of nuclear receptors are modular in nature, the LBD, DBD, and AD may be interchanged.

In another embodiment, the transcription factor comprises an AD, a DBD that recognizes a response element associated with the therapeutic protein or therapeutic polynucleotide whose expression is to be modulated; and a Group H nuclear receptor LBD. In certain embodiments, the Group H nuclear receptor LBD comprises a substitution mutation.

In another embodiment, the gene switch comprises a first transcription factor sequence, e.g., a CAP, under the control of a first therapeutic switch promoter (TSP-1) and a second transcription factor sequence, e.g., a LTF, under the control of a second therapeutic switch promoter (TSP-2), wherein the proteins encoded by said first transcription factor sequence and said second transcription factor sequence interact to form a protein complex (LDTFC), i.e., a “dual switch”- or “two-hybrid”-based gene switch. The first and second TSPs may be the same or different. In this embodiment, the presence of two different TSPs in the gene switch that are required for therapeutic molecule expression enhances the specificity of the therapeutic method.

In a further embodiment, both the first and the second transcription factor sequence, e.g., a CAP or an LTF, are under the control of a single therapeutic switch promoter (e.g., TSP-1). Activation of this promoter will generate both CAP and LTF with a single open reading frame. This can be achieved with the use of a transcriptional linker such as an IRES (internal ribosomal entry site). In this embodiment, both portions of the ligand-dependent transcription factor complex are synthesized upon activation of TSP-1. TSP-1 can be a constitutive promoter or only activated under conditions associated with the disease, disorder, or condition.

In a further embodiment, one transcription factor sequence, e.g. a LTF, is under the control of a therapeutic switch promoter only activated under conditions associated with the disease, disorder, or condition (e.g., TSP-2 or TSP-3) and the other transcription factor sequence, e.g., CAP, is under the control of a constitutive therapeutic switch promoter (e.g., TSP-1). In this embodiment, one portion of the ligand-dependent transcription factor complex is constitutively present while the second portion will only be synthesized under conditions associated with the disease, disorder, or condition.

In another embodiment, one transcription factor sequence, e.g., CAP, is under the control of a first TSP (e.g., TSP-1) and two or more different second transcription factor sequences, e.g., LTF-1 and LTF-2 are under the control of different TSPs. In this embodiment, each of the LTFs may have a different DBD that recognizes a different factor-regulated promoter sequence (e.g., DBD-A binds to a response element associated with factor-regulated promoter-1 (FRP-1) and DBD-B binds to a response element associated with factor-regulated promoter-2 (FRP-2). Each of the factor-regulated promoters may be operably linked to a different therapeutic gene. In this manner, multiple treatments may be provided simultaneously.

In one embodiment, the first transcription factor sequence encodes a polypeptide comprising a TAD (transactivation domain), a DBD (DNA binding domain) that recognizes a response element associated with the therapeutic product sequence whose expression is to be modulated; and a Group H nuclear receptor LBD (ligand binding domain), and the second transcription factor sequence encodes a transcription factor comprising a nuclear receptor LBD selected from a vertebrate retinoid X receptor (RXR), an invertebrate RXR, an ultraspiracle protein (USP), or a chimeric nuclear receptor comprising at least two different nuclear receptor ligand binding domain polypeptide fragments selected from a vertebrate RXR, an invertebrate RXR, and a USP (see WO 01/70816 A2 and US 2004/0096942 A1). The “partner” nuclear receptor ligand binding domain may further comprise a truncation mutation, a deletion mutation, a substitution mutation, or another modification.

In another embodiment, the gene switch comprises a first transcription factor sequence encoding a first polypeptide comprising a nuclear receptor LBD and a DBD that recognizes a response element associated with the therapeutic product sequence whose expression is to be modulated, and a second transcription factor sequence encoding a second polypeptide comprising an AD and a nuclear receptor LBD, wherein one of the nuclear receptor LBDs is a Group H nuclear receptor LBD. In one embodiment, the first polypeptide is substantially free of an AD and the second polypeptide is substantially free of a DBD. For purposes of the invention, “substantially free” means that the protein in question does not contain a sufficient sequence of the domain in question to provide activation or binding activity.

In another aspect of the invention, the first transcription factor sequence encodes a protein comprising a heterodimerization partner and an AD (a “CAP”) and the second transcription factor sequence encodes a protein comprising a DBD and an LBD (an “LTF”).

When only one nuclear receptor LBD is a Group H LBD, the other nuclear receptor LBD may be from any other nuclear receptor that forms a dimer with the Group H LBD. For example, when the Group H nuclear receptor LBD is an EcR LBD, the other nuclear receptor LBD “partner” may be from an EcR, a vertebrate RXR, an invertebrate RXR, an ultraspiracle protein (USP), or a chimeric nuclear receptor comprising at least two different nuclear receptor LBD polypeptide fragments selected from a vertebrate RXR, an invertebrate RXR, or a USP (see WO 01/70816 A2, International Patent Application No. PCT/US02/05235 and US 2004/0096942 A1, incorporated herein by reference in their entirety). The “partner” nuclear receptor ligand binding domain may further comprise a truncation mutation, a deletion mutation, a substitution mutation, or another modification.

In one embodiment, the vertebrate RXR LBD is from a human Homo sapiens, mouse Mus musculus, rat Rattus norvegicus, chicken Gallus, pig Sus scrofa domestica, frog Xenopus laevis, zebrafish Danio rerio, tunicate Polyandrocarpa misakiensis, or jellyfish Tripedalia cysophora RXR.

In one embodiment, the invertebrate RXR ligand binding domain is from a locust Locusta migratoria ultraspiracle polypeptide (“LmUSP”), an ixodid tick Amblyomma americanum RXR homolog 1 (“AmaRXR1”), an ixodid tick Amblyomma americanum RXR homolog 2 (“AmaRXR2”), a fiddler crab Celuca pugilator RXR homolog (“CpRXR”), a beetle Tenebrio molitor RXR homolog (“TmRXR”), a honeybee Apis mellifera RXR homolog (“AmRXR”), an aphid Myzus persicae RXR homolog (“MpRXR”), or a non-Dipteran/non-Lepidopteran RXR homolog.

In one embodiment, the chimeric RXR LBD comprises at least two polypeptide fragments selected from a vertebrate species RXR polypeptide fragment, an invertebrate species RXR polypeptide fragment, or a non-Dipteran/non-Lepidopteran invertebrate species RXR homolog polypeptide fragment. A chimeric RXR ligand binding domain for use in the present invention may comprise at least two different species RXR polypeptide fragments, or when the species is the same, the two or more polypeptide fragments may be from two or more different isoforms of the species RXR polypeptide fragment. Such chimeric RXR LBDs are disclosed, for example, in WO 2002/066614.

In one embodiment, the chimeric RXR ligand binding domain comprises at least one vertebrate species RXR polypeptide fragment and one invertebrate species RXR polypeptide fragment.

In another embodiment, the chimeric RXR ligand binding domain comprises at least one vertebrate species RXR polypeptide fragment and one non-Dipteran/non-Lepidopteran invertebrate species RXR homolog polypeptide fragment.

The ligand, when combined with the LBD of the nuclear receptor(s), which in turn are bound to the response element of an FRP associated with a therapeutic product sequence, provides external temporal regulation of expression of the therapeutic product sequence. The binding mechanism or the order in which the various components of this invention bind to each other, that is, for example, ligand to LBD, DBD to response element, AD to promoter, etc., is not critical.

In a specific example, binding of the ligand to the LBD of a Group H nuclear receptor and its nuclear receptor LBD partner enables expression of the therapeutic product sequence. This mechanism does not exclude the potential for ligand binding to the Group H nuclear receptor (GHNR) or its partner, and the resulting formation of active homodimer complexes (e.g. GHNR+GHNR or partner+partner). Preferably, one or more of the receptor domains is varied producing a hybrid gene switch. Typically, one or more of the three domains, DBD, LBD, and AD, may be chosen from a source different than the source of the other domains so that the hybrid genes and the resulting hybrid proteins are optimized in the chosen host cell or organism for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 (1988)) or LexA protein from Escherichia coli (see Brent et al., Cell 43:729 (1985)), or synthetic response elements specific for targeted interactions with proteins designed, modified, and selected for such specific interactions (see, for example, Kim et al., Proc. Natl. Acad Sci. USA, 94:3616 (1997)) to accommodate hybrid receptors. Another advantage of two-hybrid systems is that they allow choice of a promoter used to drive the gene expression according to a desired end result. Such double control may be particularly important in areas of gene therapy, especially when cytotoxic proteins are produced, because both the timing of expression as well as the cells wherein expression occurs may be controlled. When genes, operably linked to a suitable promoter, are introduced into the cells of the subject, expression of the exogenous genes is controlled by the presence of the system of this invention. Promoters may be constitutively or inducibly regulated or may be tissue-specific (that is, expressed only in a particular cell type) or specific to certain developmental stages of the organism.

The DNA binding domain of the first hybrid protein binds, in the presence or absence of a ligand, to the DNA sequence of a response element to initiate or suppress transcription of downstream gene(s) under the regulation of this response element.

The functional LDTFC, e.g., an EcR complex, may also include additional protein(s) such as immunophilins. Additional members of the nuclear receptor family of proteins, known as transcriptional factors (such as DHR38 or betaFTZ-1), may also be ligand dependent or independent partners for EcR, USP, and/or RXR. Additionally, other cofactors may be required such as proteins generally known as coactivators (also termed adapters or mediators). These proteins do not bind sequence-specifically to DNA and are not involved in basal transcription. They may exert their effect on transcription activation through various mechanisms, including stimulation of DNA-binding of activators, by affecting chromatin structure, or by mediating activator-initiation complex interactions. Examples of such co activators include RIP140, TIF1, RAP46/Bag-1, ARA70, SRC-1/NCoA-1, TIF2/GRIP/NCoA-2, ACTR/AIB1/RAC3/pCIP as well as the promiscuous coactivator C response element B binding protein, CBP/p300 (for review see Glass et al., Curr. Opin. Cell Biol. 9:222 (1997)). Also, protein cofactors generally known as corepressors (also known as repressors, silencers, or silencing mediators) may be required to effectively inhibit transcriptional activation in the absence of ligand. These corepressors may interact with the unliganded EcR to silence the activity at the response element. Current evidence suggests that the binding of ligand changes the conformation of the receptor, which results in release of the corepressor and recruitment of the above described coactivators, thereby abolishing their silencing activity. Examples of corepressors include N—CoR and SMRT (for review, see Horwitz et al., Mol Endocrinol. 10:1167 (1996)). These cofactors may either be endogenous within the cell or organism, or may be added exogenously as transgenes to be expressed in either a regulated or unregulated fashion.

In a another embodiment, an EcR-based gene switch as may be used in the present invention is described in WO 2002/066612 (PCT/US2002/005090, filed Feb. 20, 2002, published Aug. 29, 2002) which is hereby incorporated by reference in its entirety.

In additional embodiments, EcR-based gene switches that may be used in the present invention are described in WO 2001/070816 (PCT/US01/09050, filed Mar. 21, 2001, published Sep. 27, 2001); WO 2002/066614 (PCT/US02/05706, filed Feb. 20, 2002, published Aug. 29, 2002); and WO 2002/066615 (PCT/US02/05708, filed Feb. 20, 2002, published Aug. 29, 2002) each of which are hereby incorporated by reference in their entirety.

### Ligands

As used herein, the term “ligand,” as applied to ligand-activated ecdysone receptor-based gene switches are small molecules of varying solubility having the capability of activating a gene switch to stimulate expression of a polypeptide encoded therein. The ligand for a ligand-dependent transcription factor complex of the invention binds to the protein complex comprising one or more of the ligand binding domain, the heterodimer partner domain, the DNA binding domain, and the transactivation domain. The choice of ligand to activate the ligand-dependent transcription factor complex depends on the type of the gene switch utilized.

Examples of ligands include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diarylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxybenzoyl)-hydrazide), RG-115932 ((R)-3,5-Dimethyl-benzoic acid N-(1-tert-butyl-butyl)N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, PCT Appl. No. PCT/US2008/006757, and WO 2020/006274, each of which are incorporated herein by reference in their entireties.

For example, a ligand for the ecdysone receptor-based gene switch may be selected from any suitable ligands. Both naturally occurring ecdysone or ecdysone analogs (e.g., 20-hydroxyecdysone, muristerone A, ponasterone A, ponasterone B, ponasterone C, 26-iodoponasterone A, inokosterone or 26-mesylinokosterone) and non-steroid inducers may be used as a ligand for gene switch of the present invention. U.S. Pat. No. 6,379,945 BI, describes an insect steroid receptor isolated from Heliothis virescens (“HEcR”) which is capable of acting as a gene switch responsive to both steroid and certain non-steroidal inducers. Non-steroidal inducers have a distinct advantage over steroids, in this and many other systems which are responsive to both steroids and non-steroid inducers, for several reasons including, for example: lower manufacturing cost, metabolic stability, absence from insects, plants, or mammals, and environmental acceptability. U.S. Pat. No. 6,379,945 B1 describes the utility of two dibenzoylhydrazines, 1,2-dibenzoyl-1-tert-butyl-hydrazine and tebufenozide (N-(4-ethylbenzoyl)-N′-(3,5-dimethylbenzoyl)-N′-tert-butyl-hydrazine) as ligands for an ecdysone-based gene switch. Also included in the present invention as a ligand are other dibenzoylhydrazines, such as those disclosed in U.S. Pat. No. 5,117,057 B1. Use of tebufenozide as a chemical ligand for the ecdysone receptor from Drosophila melanogaster is also disclosed in U.S. Pat. No. 6,147,282. Additional, non-limiting examples of ecdysone ligands are 3,5-di-tert-butyl-4-hydroxy-N-isobutyl-benzamide, 8-O-acetylharpagide, a 1,2-diacyl hydrazine, an N′-substituted-N,N′-disubstituted hydrazine, a dibenzoylalkyl cyanohydrazine, an N-substituted-N-alkyl-N,N-diaroyl hydrazine, an N-substituted-N-acyl-N-alkyl, carbonyl hydrazine or an N-aroyl-N′-alkylN′-aroyl hydrazine. (See U.S. Pat. No. 6,723,531).

In one embodiment, the ligand for an ecdysone-based gene switch system is a diacylhydrazine ligand or chiral diacylhydrazine ligand. The ligand used in the gene switch system may be compounds of Formula I

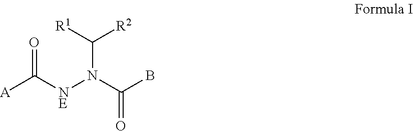

wherein A is alkoxy, arylalkyloxy or aryloxy; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; or pharmaceutically acceptable salts, hydrates, crystalline forms or amorphous forms thereof.

In another embodiment, the ligand may be enantiomerically enriched compounds of Formula II

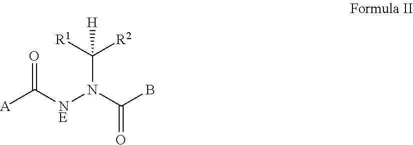

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that R1 does not equal R2; wherein the absolute configuration at the asymmetric carbon atom bearing R1 and R2 is predominantly S; or pharmaceutically acceptable salts, hydrates, crystalline forms or amorphous forms thereof.

In certain embodiments, the ligand may be enantiomerically enriched compounds of Formula III

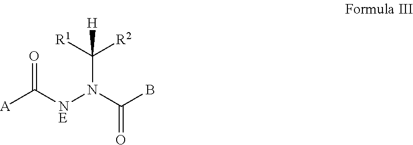

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that R1 does not equal R2; wherein the absolute configuration at the asymmetric carbon atom bearing R1 and R2 is predominantly R; or pharmaceutically acceptable salts, hydrates, crystalline forms or amorphous forms thereof.

In one embodiment, a ligand may be (R)-3,5-dimethyl-benzoic acid N-(1-tertbutyl-butyl)-N′-(2-ethyl-3-methoxy-benzoyl)-hydrazide having an enantiomeric excess of at least 95% or a pharmaceutically acceptable salt, hydrate, crystalline form or amorphous form thereof. This ligand is also referred to as veledimex.

The diacylhydrazine ligands of Formula I and chiral diacylhydrazine ligands of Formula II or III, when used with an ecdysone-based gene switch system, provide the means for external temporal regulation of expression of a therapeutic polypeptide or therapeutic polynucleotide of the present invention. See U.S. Patent Publication No. 2009/0163592, which is fully incorporated by reference herein.

The ligands used in the present invention may form salts. The term “salt(s)” as used herein denotes acidic and/or basic salts formed with inorganic and/or organic acids and bases. In addition, when a compound of Formula I, II or III contains both a basic moiety and an acidic moiety, zwitterions (“inner salts”) may be formed and are included within the term “salt(s)” as used herein. Pharmaceutically acceptable (i.e., non-toxic, physiologically acceptable) salts are used, although other salts are also useful, e.g., in isolation or purification steps which may be employed during preparation. Salts of the compounds of Formula I, II or III may be formed, for example, by reacting a compound with an amount of acid or base, such as an equivalent amount, in a medium such as one in which the salt precipitates or in an aqueous medium followed by lyophilization.

The ligands which contain a basic moiety may form salts with a variety of organic and inorganic acids. Exemplary acid addition salts include acetates (such as those formed with acetic acid or tricholoracetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The ligands which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like.

Non-limiting examples of the ligands for the inducible gene expression system utilizing the FK506 binding domain are FK506, Cyclosporin A, or Rapamycin. FK506, rapamycin, and their analogs are disclosed in U.S. Pat. Nos. 6,649,595 B2 and 6,187,757. See also U.S. Pat. Nos. 7,276,498 and 7,273,874.

A LDTF such as an EcR complex can be activated by an active ecdysteroid or non-steroidal ligand bound to one of the proteins of the complex, inclusive of EcR, but not excluding other proteins of the complex. A LDTF such as an EcR complex includes proteins which are members of the nuclear receptor superfamily wherein all members are characterized by the presence of one or more polypeptide subunits comprising an amino-terminal transactivation domain (“AD,” “TD,” or “TA,” used interchangeably herein), a DNA binding domain (“DBD”), and a ligand binding domain (“LBD”). The AD may be present as a fusion with a “heterodimerization partner” or “HP.” A fusion protein comprising an AD and HP of the invention is referred to herein as a “coactivation protein” or “CAP.” The DBD and LBD may be expressed as a fusion protein, referred to herein as a “ligand-inducible transcription factor (“LTF”). The fusion partners may be separated by a linker, e.g., a hinge region. Some members of the LTF family may also have another transactivation domain on the carboxy-terminal side of the LBD. The DBD is characterized by the presence of two cysteine zinc fingers between which are two amino acid motifs, the P-box and the D-box, which confer specificity for ecdysone response elements. These domains may be either native, modified, or chimeras of different domains of heterologous receptor proteins.

The DNA sequences making up the exogenous gene, the response element, and the LDTF, e.g., EcR complex, may be incorporated into archaebacteria, prokaryotic cells such as Escherichia coli, Bacillus subtilis, or other enterobacteria, or eukaryotic cells such as plant or animal cells. However, because many of the proteins expressed by the gene are processed incorrectly in bacteria, eukaryotic cells are preferred. The cells may be in the form of single cells or multicellular organisms. The nucleotide sequences for the exogenous gene, the response element, and the receptor complex can also be incorporated as RNA molecules, preferably in the form of functional viral RNAs such as tobacco mosaic virus. Of the eukaryotic cells, vertebrate cells are preferred because they naturally lack the molecules which confer responses to the ligands of this invention for the EcR. As a result, they are “substantially insensitive” to the ligands of this invention. Thus, the ligands useful in this invention will have negligible physiological or other effects on transformed cells, or the whole organism. Therefore, cells can grow and express the desired product, substantially unaffected by the presence of the ligand itself.

The term “ecdysone receptor complex” generally refers to a heterodimeric protein complex having at least two members of the nuclear receptor family, ecdysone receptor (“EcR”) and ultraspiracle (“USP”) proteins (see Yao et al., Nature 366:476 (1993)); Yao et al., Cell 71:63 (1992)). The functional EcR complex may also include additional protein(s) such as immunophilins. Additional members of the nuclear receptor family of proteins, known as transcriptional factors (such as DHR38, betaFTZ-1 or other insect homologs), may also be ligand dependent or independent partners for EcR and/or USP. The EcR complex can also be a heterodimer of EcR protein and the vertebrate homolog of ultraspiracle protein, retinoic acid-X-receptor (“RXR”) protein or a chimera of USP and RXR. The term EcR complex also encompasses homodimer complexes of the EcR protein or USP.

EcR ligands, when used with the EcR complex which in turn is bound to the response element linked to an exogenous gene (e.g., IL-12), provide the means for external temporal regulation of expression of the exogenous gene. The order in which the various components bind to each other, that is, ligand to receptor complex and receptor complex to response element, is not critical. Typically, modulation of expression of the exogenous gene is in response to the binding of the EcR complex to a specific control, or regulatory, DNA element. The EcR protein, like other members of the nuclear receptor family, possesses at least three domains, a transactivation domain, a DNA binding domain, and a ligand binding domain. This receptor, like a subset of the nuclear receptor family, also possesses less well-defined regions responsible for heterodimerization properties. Binding of the ligand to the ligand binding domain of EcR protein, after heterodimerization with USP or RXR protein, enables the DNA binding domains of the heterodimeric proteins to bind to the response element in an activated form, thus resulting in expression or suppression of the exogenous gene. This mechanism does not exclude the potential for ligand binding to either EcR or USP, and the resulting formation of active homodimer complexes (e.g., EcR+EcR or USP+USP). In one embodiment, one or more of the receptor domains can be varied producing a chimeric gene switch. Typically, one or more of the three domains may be chosen from a source different than the source of the other domains so that the chimeric receptor is optimized in the chosen host cell or organism for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 (1988) or LexA protein from E. coli (see Brent et al., Cell 43:729 (1985)) to accommodate chimeric EcR complexes. Another advantage of chimeric systems is that they allow choice of a promoter used to drive the exogenous gene according to a desired end result. Such double control can be particularly important in areas of gene therapy, especially when cytotoxic proteins are produced, because both the timing of expression as well as the cells wherein expression occurs can be controlled. When exogenous genes, operatively linked to a suitable promoter, are introduced into the cells of the subject, expression of the exogenous genes is controlled by the presence of the ligand of this invention. Promoters may be constitutively or inducibly regulated or may be tissue-specific (that is, expressed only in a particular cell type) or specific to certain developmental stages of the organism.

Vectors with Inducible Expression of Interleukin 12

As used herein, the terms “rAD.RheoIL12” or “Ad-RTS-hIL-12” refer to an adenoviral polynucleotide vector harboring the IL-12 gene, e.g., the human IL-12 (hIL-12) gene, under the control of a gene switch of the RheoSwitch Therapeutic System® (RTS®), which can express the IL-12 protein in the presence of activating ligand. In particular, an adenoviral vector (e.g. the Ad-RTS-hIL-12 viral vector) encodes (i) a first polynucleotide encoding an IL-12 p40 polypeptide comprising an amino acid sequence, e.g., at least 75%, 80%, 85%, 90%, 91%, 92%, 93%, 94%, 95%, 96%, 97%, 98%, 99%, 100%, or any percentage in between, identical to wild-type human IL-12 p40 polypeptide (e.g., the amino acid sequence of SEQ ID NO: 1 or amino acids 23-328 of SEQ ID NO: 1); (ii) a second polynucleotide encoding an IL-12 p35 polypeptide comprising an amino acid sequence, e.g., at least 75%, 80%, 85%, 90%, 91%, 92%, 93%, 94%, 95%, 96%, 97%, 98%, 99%, 100%, or any percentage in between, identical to wild-type human IL-12 p35 polypeptide (e.g., the amino acid sequence of SEQ ID NO: 2 or amino acids 23-219 of SEQ ID NO: 2); (iii) a third polynucleotide encoding a VP-16 transactivation domain-retinoic acid-X-receptor fusion protein (VP-16-RXR), e.g., comprising an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 3 to or amino acids 2-324 of SEQ ID NO: 3; and (iv) a fourth polynucleotide encoding a Gal4 DNA binding domain and an ecdysone receptor (EcR) binding domain fusion protein (Gal4-EcR), e.g., comprising an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 4 or to amino acids 2-488 of SEQ ID NO: 4, wherein the VP-16-RXR fusion protein and the Gal4-EcR fusion protein form a ligand dependent transcription factor complex. The activating ligand is a diacylhydrazine ligand such as veledimex.

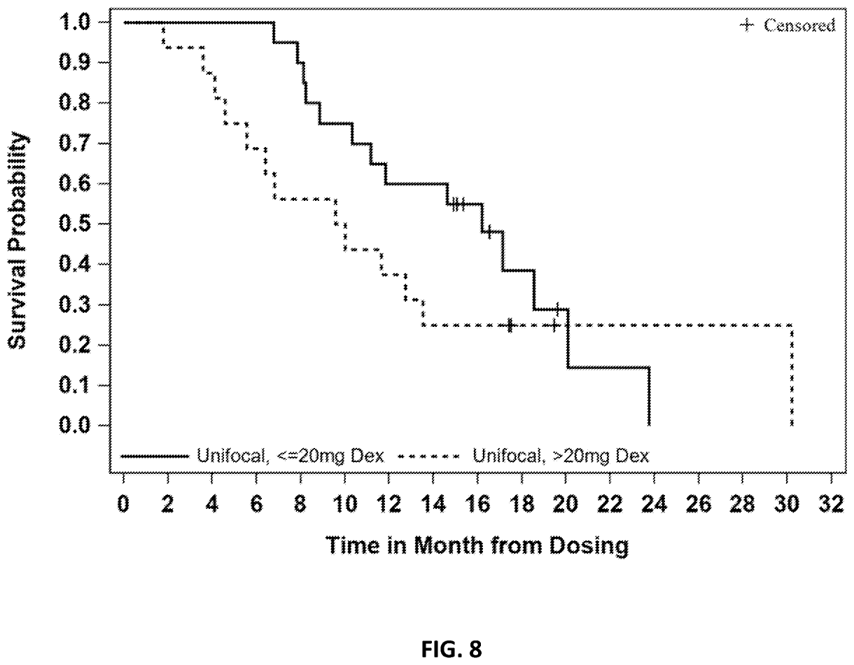

The recombinant DNA used as the recombinant adenoviral vector allows the expression of human IL-12 under the control of the RheoSwitch Therapeutic System® (RTS®). In some embodiments, the RTS® comprises a bicistronic message expressed from the human Ubiquitin C promoter and codes for two fusion proteins: Gal4-EcR and VP16-RXR. In some embodiments, Gal4-EcR is a fusion between the DNA binding domain (amino acids 1-147) of yeast Gal4 and the DEF domains of the ecdysone receptor from the insect Choristoneura fumiferana. In some embodiments, VP16-RXR is a fusion between the transcription activation domain of HSV-VP16 and the EF domains of a chimeric RXR derived from human and locust sequences. In some embodiments, these Gal4-EcR and VP16-RXR sequences are separated by an internal ribosome entry site (IRES) from EMCV. In some embodiments, these two fusion proteins dimerize when Gal4-EcR binds to a small molecule drug (RG-115932) and activate transcription of hIL-12 from a Gal4-responsive promoter that contains six Gal4-binding sites and a synthetic minimal promoter. In some embodiments, the RTS transcription unit described above is placed downstream of the hIL-12. In some embodiments, the RTS-hIL12 cassette is incorporated into the adenovirus 5 genome at the site where the E1 region has been deleted. In some embodiments, the adenoviral backbone also lacks the E3 gene. A map for the adenoviral vector Ad-RTS-hIL-12 is shown in FIG. 8 of US 2009/0123441 A1, which is incorporated by reference in its entirety.

In some embodiments, the IL-12 p40 polypeptide of the disclosure comprises the amino acid sequence of:

(SEQ ID NO: 1)
MGHQQLVISWESLVELASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTESVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS

In some embodiments, the IL-12 p40 polypeptide of the disclosure comprises amino acids 23-328 of SEQ ID NO: 1. In some embodiments, the IL-12 p40 polypeptide of the disclosure comprises an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 1. In some embodiments, the IL-12 p40 polypeptide of the disclosure comprises an amino acid sequence at least 75%, 80%, 85%, 90%, 91%, 92%, 93%, 94%, 95%, 96%, 97%, 98%, 99%, 100%, or any percentage in between, identical to amino acids 23-328 of SEQ ID NO: 1.

In some embodiments, the IL-12 p35 polypeptide of the disclosure comprises the amino acid sequence of:

(SEQ ID NO: 2)
MGPARSLLLVATLVLLDHLSLARNLPVATPDPGMFPCLHHSQNLLRAVS
NMLQKARQTLEFYPCTSEEIDHEDITKDKISTVEACLPLELTKNESCLN
SRETSFITNGSCLASRKTSEMMALCLSSIYEDLKMYQVEFKTMNAKLLM
DPKRQIFLDQNMLAVIDELMQALNENSETVPQKSSLEEPDFYKTKIKLC
ILLHAFRIRAVTIDRVMSYLNAS

In some embodiments, the IL-12 p35 polypeptide of the disclosure comprises amino acids 23-219 of SEQ ID NO: 2. In some embodiments, the IL-12 p35 polypeptide of the disclosure comprises an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 2. In some embodiments, the IL-12 p35 polypeptide of the disclosure comprises an amino acid sequence at least 75%, 80%, 85%, 90%, 91%, 92%, 93%, 94%, 95%, 96%, 97%, 98%, 99%, 100%, or any percentage in between, identical to amino acids 23-219 of SEQ ID NO: 2.

As used herein, the term “IL-12p70” refers to IL-12 protein, which naturally has two subunits commonly referred to as p40 and p35. IL-12p70 is a heterodimeric cytokine that includes the p35 alpha chain, e.g., any of the IL-12 p35 polypeptides disclosed herein, and the p40 beta chain, e.g., any of the IL-12 p40 polypeptides described herein. The term IL-12p70 encompasses fusion proteins comprising the two subunits of IL-12 (IL-12 p40 and IL-12 p35), wherein the fusion protein may include linker amino acids between subunits.

In some embodiments, the VP16-RXR fusion protein of the disclosure comprises the amino acid sequence of:

(SEQ ID NO: 3)
MGPKKKRKVAPPTDVSLGDELHLDGEDVAMAHADALDDEDLDMLGDGDS
PGPGETPHDSAPYGALDMADFEFEQMETDALGIDEYGGEFEMPVDRILE
AELAVEQKSDQGVEGPGGTGGSGSSPNDPVTNICQAADKQLFTLVEWAK
RIPHFSSLPLDDQVILLRAGWNELLIASFSHRSIDVRDGILLATGLHVH
RNSAHSAGVGAIFDRVLTELVSKMRDMRMDKTELGCLRAIILENPEVRG
LKSAQEVELLREKVYAALEEYTRTTHPDEPGRFAKLLLRLPSLRSIGLK
CLEHLFFFRLIGDVPIDTFLMEMLESPSDS

In some embodiments, the VP16-RXR fusion protein of the disclosure comprises amino acids 2-324 of SEQ ID NO: 3. In some embodiments, the VP16-RXR fusion protein of the disclosure comprises an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 3. In some embodiments, the VP16-RXR fusion protein of the disclosure comprises an amino acid sequence at least 75%, 80%, 85%, 90%, 91%, 92%, 93%, 94%, 95%, 96%, 97%, 98%, 99%, 100%, or any percentage in between, identical to amino acids 2-324 of SEQ ID NO: 3.

In some embodiments, the Gal4-EcR fusion protein of the disclosure comprises the amino acid sequence of:

(SEQ ID NO: 4)
MKLLSSIEQACDICRLKKLKCSKEKPKCAKCLKNNWECRYSPKTKRSPL
TRAHLTEVESRLERLEQLFLLIFPREDLDMILKMDSLQDIKALLTGLFV
QDNVNKDAVTDRLASVETDMPLTLRQHRISATSSSEESSNKGQRQLTVS
PEFPGIRPECVVPETQCAMKRKEKKAQKEKDKLPVSTTTVDDHMPPIMQ
CEPPPPEAARIHEVVPRELSDKLLVTNRQKNIPQLTANQQFLIARLIWY
QDGYEQPSDEDLKRITQTWQQADDENEESDTPFRQITEMTILTVQLIVE
FAKGLPGFAKISQPDQITLLKACSSEVMMLRVARRYDAASDSILFANNQ
AYTRDNYRKAGMAEVIEDLLHFCRCMYSMALDNIHYALLTAVVIFSDRP
GLEQPQLVEEIQRYYLNTLRIYILNQLSGSARSSVIYGKILSILSELR
TLGMQNSNMCISLKLKNRKLPPFLEEIWDVADMSHTQPPPILESPTNL

In some embodiments, the Gal4-EcR fusion protein of the disclosure comprises amino acids 2-488 of SEQ ID NO: 4. In some embodiments, the Gal4-EcR fusion protein of the disclosure comprises an amino acid sequence at least 75%, 80%, 85%, 90%, 91%, 92%, 93%, 94%, 95%, 96%, 97%, 98%, 99%, 100%, or any percentage in between, identical to the amino acid sequence of SEQ ID NO: 4. In some embodiments, the Gal4-EcR fusion protein of the disclosure comprises an amino acid sequence at least 75%, 80%, 85%, 90%, 91%, 92%, 93%, 94%, 95%, 96%, 97%, 98%, 99%, 100%, or any percentage in between, identical to amino acids 2-488 of SEQ ID NO: 4.

In one embodiment, the recombinant adenoviral vector contains the following exemplary regulatory elements in addition to the viral vector sequences: Human Ubiquitin C promoter, Internal ribosome entry site derived from EMCV, an inducible promoter containing 6 copies of Gal4-binding site, 3 copies of SP-1 binding sites and a synthetic minimal promoter sequence, SV40 polyadenylation sites and a transcription termination sequence derived from human alpha-globin gene.

In one embodiment, the recombinant adenoviral vector Ad-RTS-hIL-12 is produced in the following manner. The coding sequences for the receptor fusion proteins, VP16-RXR and Gal4-EcR separated by the EMCV-IRES (internal ribosome entry site), are inserted into the adenoviral shuttle vector under the control of the human ubiquitin C promoter (constitutive promoter). Subsequently, the coding sequences for the p40 and p35 subunits of hIL-12 separated by IRES is placed under the control of a synthetic inducible promoter containing 6 copies of Gal4-binding site inserted upstream of the ubiquitin C promoter and the receptor sequences. The shuttle vector contains the adenovirus serotype 5 sequences from the left end to map unit 16 (mu16), from which the E1 sequences are deleted and replaced by the RTS and IL-12 sequences (RTS-hIL-12). The shuttle vector carrying the RTS-hIL12 is tested by transient transfection in HT-1080 cells for Activator Drug-dependent IL-12 expression. The shuttle vector is then recombined with the adenoviral backbone by cotransfection into HEK 293 cells to obtain recombinant adenovirus Ad-RTS-hIL-12. The adenoviral backbone contains sequence deletions of mu 0 to 9.2 at the left end of the genome and the E3 gene. The shuttle vector and the adenoviral backbone contain the overlapping sequence from mu 9.2 to mu 16 that allows the recombination between them and production of the recombinant adenoviral vector. Since the recombinant adenoviral vector is deficient in the E1 and E3 regions, the virus is replication-deficient in normal mammalian cells. However, the virus can replicate in HEK 293 cells that harbor the adenovirus-5 E1 region and hence provide the E1 function in trans-complementation.

In certain embodiments, Ad-RTS-hIL-12 and components thereof are encoded by polynucleotide and polypeptide sequences as described and disclosed in:

SEQ ID NOs: 1-64 in WO2001/070816 (PCT/US2001/09050) filed 21 Mar. 2001;

SEQ ID NOs: 1-113 in WO2002/066612 (PCT/US2002/005090) filed 20-Feb. 2002;

SEQ ID NOs: 1-75 in WO2002/066614 (PCT/US2002/005706) filed 20-Feb. 2002;

SEQ ID NOs: 1-8 and 13 in WO2009/048560 (PCT/US2008/011563) filed 08-Oct. 2008;

SEQ ID NOs: 1-24 and 29 in WO2010/042189 (PCT/US2009/005510) filed 8 Oct. 2009; and,

SEQ ID NOs: 1-6, 24-29, 47-62 in WO2011/119773 (PCT/US2011/029682) filed 23 Mar. 2011.

The disclosure and sequences from the sequence listings in each of the above referenced publications are hereby incorporated by reference in their entireties. These embodiments are also disclosed in WO 2020/006274, incorporated by reference herein in its entirety.

In some embodiments, a nucleic acid adenoviral vector is provided containing a gene switch, wherein the coding sequences for VP16-RXR and Gal4-EcR are separated by the EMCV internal ribosome entry site (IRES) sequence and are inserted into the adenoviral shuttle vector under the control of the human ubiquitin C promoter. In some embodiments, the coding sequences for the p40 and p35 subunits of IL-12 separated by an IRES sequence and placed under the control of a synthetic inducible promoter, are inserted upstream of the ubiquitin C promoter.

In some embodiments, the invention provides a shuttle vector carrying transcription units (VP16-RXR and Gal4-EcR) for the two fusion proteins and inducible IL-12 subunits recombined with the adenoviral backbone (AdEasyl) in E. coli BJ5183 cells. After verifying the recombinant clone, the plasmid carrying the rAd.RheoIL12 genome is grown in and purified from XL10-Gold cells, digested off the plasmid backbone and packaged by transfection into HEK 293 cells or CHO cells or other suitable cell lines.

Purification of the vector to enhance the concentration can be accomplished by any suitable method, such as by density gradient purification (e.g., cesium chloride (CsCl)) or by chromatography techniques (e.g., column or batch chromatography). For example, the vector of the invention can be subjected to two or three CsCl density gradient purification steps. In some embodiments, the vector, e.g., a replication-deficient adenoviral vector, is purified from cells infected with the replication-deficient adenoviral vector using a method that comprises lysing cells infected with adenovirus, applying the lysate to a chromatography resin, eluting the adenovirus from the chromatography resin and collecting a fraction containing adenovirus.

In some embodiments, the resulting primary viral stock is amplified by re-infection of HEK293 cells or CHO cells or other suitable cell lines and is purified by CsCl density-gradient centrifugation or other suitable purification methods.

In some embodiments, the IL-12 gene is a wild-type gene sequence. In another embodiment, the IL-12 gene is a modified gene sequence, e.g., a chimeric sequence or a sequence that has been modified to use preferred codons.

### Methods of Treatment

In some embodiments, the therapeutic methods of the invention involve in vivo introduction of an adenoviral vector, e.g., an Ad-RTS-hIL-12 viral vector, (e.g., a therapeutically effective amount of an adenoviral vector, e.g., an Ad-RTS-hIL-12 viral vector) into a subject. In some embodiments, the subject has cancer. In some embodiments, the cancer is a glioma. In some embodiments, the glioma is a glioblastoma. In some embodiments, the glioblastoma is grade III or grade IV malignant glioma. In some embodiments, the glioblastoma may be primary glioblastoma, secondary glioblastoma, recurrent glioblastoma or progressive glioblastoma. The glioblastoma may be unifocal glioblastoma or multifocal glioblastoma. In some embodiments, the subject is a human. In some embodiments, unifocal glioblastoma is shown through the identification of one enhancing lesion in MRI. In some embodiments, multifocal glioblastoma is shown through the identification of multiple enhancing lesions in MRI.

In some embodiments, the subject is at least 18 years old and at most 75 years old. In some embodiments, the subject has not received nitrosoureas for 6 weeks. In some embodiments, the subject has not received other cytotoxic agents for 4 weeks. In some embodiments, the subject has not received antiangiogenic agents including bevacizumab for 4 weeks. In some embodiments, the subject has not received small-molecule tyrosine kinase inhibitors for 2 weeks. In some embodiments, the subject has not received a PD-1 or CTLA-4 antagonist for 3 months. In some embodiments, the subject has not received vaccine-based therapy in 3 months. In some embodiments, the subject has a Karnofsky performance status of greater than or equal to 70. In some embodiments, the subject has a hemoglobin concentration ≥9 g/L. In some embodiments, the subject has lymphocytes at a concentration of >500/mm³. In some embodiments, the subject has an absolute neutrophil count ≥1,500/mm³. In some embodiments, the subject has a platelet count ≥100,000/mm³. In some embodiments, the subject has a serum creatine concentration less than or equal to 1.5× the upper limit of normal (ULN). In some embodiments, the subject has an aspartate transaminase and alanine transaminase concentrations greater than or equal to 2.5×ULN. In some embodiments, the subject has an aspartate transaminase and alanine transaminase concentrations greater than or equal to 5×ULN when the subject has documented liver metastases. In some embodiments, the subject has a total bilirubin concentration less than 1.5×ULN.

In some embodiments, the subject has not had radiotherapy treatment in 4 weeks prior to treatment. In some embodiments, the subject does not have clinically significant increased intracranial pressure or uncontrolled seizures. In some embodiments, the subject does not have known immunosuppressive disease, autoimmune conditions, and/or chronic viral infections (e.g., human immunodeficiency virus (HIV), or hepatitis. In some embodiments, the subject does not use systemic antibacterial, antifungal, or antiviral medication for the treatment of acute clinically significant infection within 2 weeks of first veledimex dose. In some embodiments, the subject is not febrile prior to Ad-RTS-hIL-12 injection. In some embodiments, the subject does not use enzyme-inducing antiepileptic drugs (EIAED) within 7 days prior to the first dose of study drug. In some embodiments, the subject does have concurrent clinically active malignant disease, requiring treatment, with the exception of non-melanoma cancers of the skin or carcinoma in situ of the cervix or nonmetastatic prostate cancer. In some embodiments, the subject is not a Nursing or pregnant female. In some embodiments, the subject does not have prior exposure to veledimex. In some embodiments, the subject does not use medications that induce, inhibit, or are substrates of cytochrome p450 (CYP450) 3A4 within 7 days prior to veledimex dosing. In some embodiments, the subject does not have any contraindication for a neurosurgical procedure.

In some embodiments, the adenoviral vector, e.g., the Ad-RTS-hIL-12 viral vector, is introduced into the subject systemically or locally. In some embodiments, an adenoviral vector, e.g., the Ad-RTS-hIL-12 viral vector, is introduced intratumorally, at the site of the tumor. In some embodiments, the adenoviral vector, e.g., the Ad-RTS-hIL-12 viral vector, is injected into the glioblastoma or periphery thereof or into a resection site of the glioblastoma or periphery thereof. In some embodiments, the adenoviral vector, e.g., the Ad-RTS-hIL-12 viral vector, is injected intraoperatively into a cavity wall immediately following glioblastoma resection. In some embodiments, the adenoviral vector, e.g., the Ad-RTS-hIL-12 viral vector, is administered directly to the tumor during craniotomy for resection of the tumor. Alternatively, the adenoviral vector, e.g., the Ad-RTS-hIL-12 viral vector, is administered stereotactically.

An effective amount of an adenoviral vector, e.g., an Ad-RTS-hIL-12 viral vector, is a unit dose of about 1×10¹¹, 2×10¹¹, 3×10¹¹, 4×10¹¹, 5×10¹¹, 6×10¹¹, 7×10¹¹, 8×10¹¹, 9×10¹¹, 1×10¹² or 2×10¹² viral particles (vp). Preferably, the viral vector is administered at a unit dose of 2×10¹¹ vp.

In some embodiments, the vector may be delivered by injection. In some embodiments, direct administration to the tumor, tumor site or lymph node includes intratumoral injection, e.g., injection into an existing tumor or the periphery thereof or injection into a tumor resection site or the periphery thereof (e.g., injection intraoperatively into the cavity wall immediately following tumor resection), of a liquid pharmaceutical composition via syringe. In some embodiments, direct administration may involve injection via a cannula or other suitable instrument for delivery for a vector. In some embodiments, direct administration may comprise an implant further comprising a suitable vector for delivery of transgenes such as IL-12. In some embodiments, the implant may be either directly implanted in or near the tumor.

In some embodiments, the adenoviral vector, e.g., the Ad-RTS-hIL-12 viral vector, is administered as a single administration or multiple administration, e.g., two, three, four or more administrations.

In some embodiments, expression of the polypeptide (e.g. IL-12) by the polynucleotide is induced by administration of a diacylhydrazine ligand as described herein to the subject. In some embodiments, the ligand is veledimex.

In some embodiments, the ligand is administered by any suitable method, either systemically (e.g., orally or intravenously) or locally (e.g., intraperitoneally, intrathecally, intraventricularly, direct injection into the tissue or organ where the disease or disorder is occurring, e.g., intratumorally). In some embodiments, the ligand is administered orally.

In some embodiments, the ligand is administered at a unit daily dose of about 1 mg to about 120 mg. In some embodiments, the ligand is administered at a unit daily dose of about 5, 10, 15, 20, 25, 30, 35, 40, 50, 60, 70, 80, 90, 100 or 120 mg. In some embodiments, the ligand is administered at a unit daily dose of about 5 mg, 10 mg, 15 mg, 20 mg, 40 mg, 80 mg or 120 mg. In some embodiments, the ligand is administered at a unit daily dose of about 20 mg.

In some embodiments, the ligand is administered once a day, twice a day or every other day. Preferably the ligand is administered once daily.

In some embodiments, a corticosteroid is administered during the administration of the ligand. In some embodiments, the cumulative dose of corticosteroid during the administration of ligand is less than or equal to about 5 mg, 10 mg, 15 mg, 20 mg, 25 mg 30 mg. In some embodiments, the cumulative dose is less than or equal to 20 mg. In some embodiments, no corticosteroid is administered.

In some embodiments, the corticosteroid is administered orally or parentally. In some embodiments, the corticosteroid is administered intravenously.

The term “subject,” or “individual” or “patient” as used herein is in reference to individuals having a disease or disorder or are suspected of having a disease or disorder and the like. Subject, individual or patent may be used interchangeably in the disclosure. The subject is a human subject. The subject is a pediatric patient or an adult patient.

In some embodiments, the subject has never previously been administered a corticosteroid. In some embodiments, the subject has been previously administered a corticosteroid. In some embodiments, the subject was not administered a corticosteroid for treatment of the underlying disease or concurrent symptoms for 4 weeks prior to the administration of the ligand. Alternatively, the subject was administered a corticosteroid for treatment of the underlying disease or concurrent symptoms within 4 weeks prior to the administration of the ligand. In some embodiments, the subject was not administered a corticosteroid for 4 weeks prior to the administration of the ligand. Alternatively, the subject was administered a corticosteroid within 4 weeks prior to the administration of the ligand.

These and additional embodiments are also disclosed in WO 2020/006274, incorporated by reference herein in its entirety.

### Dosing Regimens

The invention provides dosing regimens for treating a subject having glioblastoma with an adenoviral vector, e.g., an Ad-RTS-hIL-12 vector, a ligand (e.g., veledimex) and optionally a corticosteroid. The dosage amounts of the corticosteroid are described herein supra.

In some embodiments, the initial dose of the adenoviral vector, e.g., the Ad-RTS-hIL-12 viral vector, and the initial dose of the ligand (e.g., a therapeutically effective amount of each) are administered concurrently or sequentially. In some embodiments, the initial dose of the ligand is administered at a period of time after the initial dose of the vector. In some embodiments, initial dose of the ligand is administered at a period of time prior to the initial dose of the vector. In some embodiments, the initial dose of the ligand is administered at about 1, 2, 3, 4, 5, 6, 7, 8, 9, 10, 11 or 12 hours prior to the administration of the vector. In some embodiments, one or more subsequent doses of the ligand are administered once daily after the administration of the initial dose of the ligand. In other embodiments, the one or more subsequent doses of the ligand are administered once daily for 3-28 days after the administration of the initial dose of the ligand. In some embodiments, daily subsequent doses of the ligand are administered for 3, 4, 5, 6, 7, 8, 9, 10, 11, 12, 13, 14, 15, 16, 17, 18, 19, 20, 21, 22, 23, 24, 25, 26, 27, 28 or more days after the after the administration of the initial dose of the ligand. In some embodiments, the ligand is administered daily for 14 days after the after the administration of the initial dose of the ligand. In some embodiments, a corticosteroid is further administered to the subject during the treatment period of the ligand. In some cases, administration of corticosteroids is for treatment of postsurgical cerebral edema. In some embodiments, a corticosteroid is not administered to the subject during the treatment period of the ligand.

In a certain embodiment, the viral vector is administered by injection and then the subject starts oral veledimex for 14 days.

In some embodiments, the initial dose of the vector and the initial dose of the corticosteroid. For example, the initial dose of the vector after the initial dose of the corticosteroid. Alternatively, initial dose of period of time before to the initial dose of the corticosteroid. In some embodiments the initial dose of the corticosteroid is administered at about 1, 2, 3, 4, 5, 6, 7 or more days prior to the administration of the vector. In some embodiments corticosteroid are administered vector. For example, one or more subsequent doses of the corticosteroid are administered within 7 to 28 days after the administration of the vector. In some. one or more subsequent doses of the corticosteroid are administered embodiments at least 7, 8, 9, 10, 11, 12, 13, 14, 15, 16, 17, 18, 19, 20, 21, 22, 23, 24, 25, 26, 27, 28 or more days after Preferably, one of the subsequent doses of the corticosteroid are administered embodiments at 15 days after administration of the vector.

In other embodiments, subsequent doses of the corticosteroid are administered once every one, two, three or four weeks after the first subsequent dose of the corticosteroid. In some embodiments, subsequent doses of the corticosteroid are administered once every two week or once every four weeks after the first subsequent dose of the corticosteroid.

In some embodiments, the initial dose of the ligand and the initial dose of the corticosteroid are administered concurrently or sequentially. For example, the initial dose of the ligand is administered at a period of time after the initial dose of the corticosteroid. Alternatively, initial dose of the ligand is administered at a period of time before to the initial dose of the corticosteroid. In some embodiments the initial dose of the corticosteroid is administered at about 1, 2, 3, 4, 5, 6, 7 or more days prior to the administration of the ligand. In some embodiments one or more subsequent doses of the corticosteroid are administered after the administration of the initial dose of the ligand. For example, one or more subsequent doses of the corticosteroid are administered within 7 to 28 days after the administration of the ligand. In some embodiments, one or more subsequent doses of the corticosteroid are administered at least 7, 8, 9, 10, 11, 12, 13, 14, 15, 16, 17, 18, 19, 20, 21, 22, 23, 24, 25, 26, 27, 28 or more days after administration of the ligand. In some embodiments, one of the subsequent doses of the corticosteroid are administered 15 days after administration of the ligand.

In other embodiments, subsequent doses of the corticosteroid are administered once every one, two, three or four weeks after the first subsequent dose of the corticosteroid. Preferably, subsequent doses of the corticosteroid are administered once every two week or once every four weeks after the first subsequent dose of the corticosteroid.

These and additional embodiments are also disclosed in WO 2020/006274, incorporated by reference herein in its entirety.

### Pharmaceutical Compositions

The viral vectors, ligands, and corticosteroids described herein (also referred to herein as “therapeutic compound(s)”), can be incorporated into pharmaceutical compositions suitable for administration. Such compositions typically include the therapeutic compound(s) and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, (e.g., intravenous, intradermal, subcutaneous) oral (including, inhalation), topical; (i.e., transdermal), transmucosal, or rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous or intratumoral administration, suitable carriers include physiological saline, bacteriostatic water, Cremophor ELT™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the therapeutic compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the therapeutic compound(s) are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The therapeutic compound(s) can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of therapeutic compound(s) calculated to produce the desired therapeutic effect in association with the required pharmaceutical carrier. The specification for the dosage unit forms of the disclosure are dictated by and directly dependent on the unique characteristics of the therapeutic compound(s) and the particular therapeutic effect to be achieved, and the limitations inherent in the art of compounding such a therapeutic compound(s) for the treatment of individuals.

The pharmaceutical compositions can be included in a container, pack, or dispenser together with instructions for administration.

These and additional embodiments are also disclosed in WO 2020/006274, incorporated by reference herein in its entirety.

### Definitions

The term “isolated” for the purposes of the invention designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

The term “purified,” as applied to biological materials does not require the material to be present in a form exhibiting absolute purity, exclusive of the presence of other compounds. It is rather a relative definition.

“Nucleic acid,” “nucleic acid molecule,” “oligonucleotide,” “nucleotide,” and “polynucleotide” are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine or deoxy cytidine; “DNA molecules”) or any phosphoester analogs thereof, such as phosphorothioates and thioesters, in either single stranded form or a double-stranded helix. Double stranded DNA-DNA, DNA-RNA and RNA-RNA helices are possible. The term nucleic acid molecule and in particular DNA or RNA molecule, refers only to the primary and secondary structure of the molecule and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA and semi-synthetic DNA.

The term “fragment,” as applied to polynucleotide sequences, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000 or more consecutive nucleotides of a nucleic acid according to the invention.

As used herein, an “isolated nucleic acid fragment” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to a polynucleotide comprising nucleotides that encode a functional molecule, including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism, A “foreign” gene or “heterologous” gene refers to a gene not normally found in the host organism, but that is introduced into the host organism by gene transfer. Foreign genes can comprise native genes inserted into a non-native organism or chimeric genes. A “transgene” is a gene that has been introduced into the genome by a transformation procedure. For example, the interleukin-12 (IL-12) gene encodes the IL-12 protein. IL-12 is a heterodimer of a 35-kD subunit (p35) and a 40-kD subunit (p40) linked through a disulfide linkage to make fully functional IL-12p70. The IL-12 gene encodes both the p35 and p40 subunits.

“Heterologous DNA” refers to DNA not naturally located in the cell or in a chromosomal site of the cell. The heterologous DNA may include a gene foreign to the cell.

The term “genome” includes chromosomal as well as mitochondrial, chloroplast and viral DNA or RNA.

A nucleic acid molecule is “hybridizable” to another nucleic acid molecule, such as a cDNA, genomic DNA or RNA, when a single stranded form of the nucleic acid molecule can anneal to the other nucleic acid molecule under the appropriate conditions of temperature and solution ionic strength. Hybridization and washing conditions are well known and exemplified in Sambrook et al. in Molecular Cloning: A Laboratory Manual, Second Edition, Cold Spring Harbor Laboratory Press, Cold Spring Harbor (1989), particularly Chapter 11 and Table 11.1 therein). The conditions of temperature and ionic strength determine the “stringency” of the hybridization.

Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a Tₘ of 55°, can be used, e.g., 5×SSC, 0.1% SDS, 0.25% milk and no formamide; or 30% formamide, 5×SSC, 0.5% SDS. Moderate stringency hybridization conditions correspond to a higher Tm, e.g., 40% formamide, with 5× or 6×SSC. High stringency hybridization conditions correspond to the highest Tm, e.g., 50% formamide, 5× or 6×SSC.

Hybridization requires that the two nucleic acids contain complementary sequences, although depending on the stringency of the hybridization, mismatches between bases are possible. The term “complementary” is used to describe the relationship between nucleotide bases that are capable of hybridizing to one another. For example, with respect to DNA, adenosine is complementary to thymine and cytosine is complementary to guanine. Accordingly, the invention also includes isolated nucleic acid fragments that are complementary to the complete sequences as disclosed or used herein as well as those substantially similar nucleic acid sequences.

In one embodiment of the invention, polynucleotides are detected by employing hybridization conditions comprising a hybridization step at Tₘ of 55° C. and utilizing conditions as set forth above. In other embodiments, the Tm is 60° C., 63° C. or 65° C.

In some embodiments, post-hybridization washes also determine stringency conditions. One set of conditions uses a series of washes starting with 6×SSC, 0.5% SDS at room temperature for 15 minutes (min), then repeated with 2×SSC, 0.5% SDS at 45° C. for 30 min and then repeated twice with 0.2×SSC, 0.5% SDS at 50° C. for 30 min. One set of stringent conditions uses higher temperatures in which the washes are identical to those above except for the temperature of the final two 30 min washes in 0.2×SSC, 0.5% SDS is increased to 60° C. Another set of highly stringent conditions uses two final washes in 0.1×SSC, 0.1% SDS at 65° C.

The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important and the length of the oligonucleotide determines its specificity (see Sambrook et al., supra, 11.7-11.8).

In one embodiment of the invention, polynucleotides are detected by employing hybridization conditions comprising a hybridization step in less than 500 mM salt and at least 37° C. and a washing step in 2×SSPE at a temperature of at least 63° C. In another embodiment, the hybridization conditions comprise less than 200 mM salt and at least 37° C. for the hybridization step. In a further embodiment, the hybridization conditions comprise 2×SSPE and 63° C. for both the hybridization and washing steps.

In another embodiment, the length for a hybridizable nucleic acid is at least about 10 nucleotides. Preferably a minimum length for a hybridizable nucleic acid is at least about 15 nucleotides; e.g., at least about 20 nucleotides; e.g., at least 30 nucleotides. Furthermore, the skilled artisan will recognize that the temperature and wash solution salt concentration may be adjusted as necessary according to factors such as length of the probe.

The term “probe” refers to a single-stranded nucleic acid molecule that can base pair with a complementary single stranded target nucleic acid to form a double-stranded molecule.

A “primer” refers to an oligonucleotide that hybridizes to a target nucleic acid sequence to create a double stranded nucleic acid region that can serve as an initiation point for DNA synthesis under suitable conditions. Such primers may be used in a polymerase chain reaction or for DNA sequencing.

“Polymerase chain reaction” is abbreviated PCR and refers to an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and refers to an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within or downstream (3′ non-coding sequences) of a coding sequence and which influence the transcription, RNA processing or stability or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and refers to a length of nucleic acid sequence, either DNA, cDNA or RNA, that comprises a translation start signal or initiation codon, such as an ATG or AUG and a termination codon and can be potentially translated into a polypeptide sequence.

The term “head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head” may be abbreviated (5′)-to-(5′) and may also be indicated by the symbols (← →) or (3′←5′5′→3′).

The term “tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′) and may also be indicated by the symbols (→ ←) or (5′→3′3′←5′).

The term “head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5′)-to-(3′) and may also be indicated by the symbols (→→) or (5′→3′5′→κ3′).

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

The terms “restriction endonuclease” and “restriction enzyme” are used interchangeably and refer to an enzyme that binds and cuts within a specific nucleotide sequence within double stranded DNA.

“Homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology and greater degrees of sequence similarity, may increase the efficiency of homologous recombination.

Several methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda) and plasmid and cosmid DNA. vectors, to name but a few.

A “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both, viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives or plasmids such as pBR322 or pUC plasmid derivatives or the Bluescript vector. Another example of vectors that are useful in the invention is the ULTRAVECTOR® Production System (Intrexon Corp., Blacksburg, VA) as described in WO 2007/038276, incorporated by reference herein in its entirety. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia virus, herpes simplex virus, Epstein-Barr virus, adenovirus, geminivirus and caulimovirus vectors. Non-viral vectors include plasmids, liposomes, electrically charged lipids (cytofectins), DNA-protein complexes and biopolymers. In addition to a nucleic acid, a vector may also comprise one or more regulatory regions, and/or selectable markers useful in selecting, measuring and monitoring nucleic acid transfer results (transfer to which tissues, duration of expression, etc.).

The term “plasmid” refers to an extra-chromosomal element often carrying a gene that is not part of the central metabolism of the cell and usually in the form of circular double-stranded DNA molecules. Such elements may be autonomously replicating sequences, genome integrating sequences, phage or nucleotide sequences, linear, circular or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” refers to a “replicon,” which is a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

The term “expression vector” refers to a vector, plasmid or vehicle designed to enable the expression of an inserted nucleic acid sequence. The cloned gene, i.e., the inserted nucleic acid sequence, is usually placed under the control of control elements such as a promoter, a minimal promoter, an enhancer or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC1, HIS3, GALL, GAL4, GAL10, ADH1, PGK, PH05, GAPDH, ADC1, TRP1, URA3, LEU2, ENO, TPI, alkaline phosphatase promoters (useful for expression in Saccharomyces); AOX1 promoter (useful for expression in Pichia); β-lactamase, lac, ara, tet, trp, IPL, IPR, T7, tac and trc promoters (useful for expression in Escherichia coli); light regulated-, seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus IE1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, ct-actin, tubulin and the like), the promoters of the intermediate filaments (desmin, neurofilaments, keratin, GFAP and the like), the promoters of therapeutic genes (of the MDR, CFTR or factor VIII type and the like), pathogenesis or disease related-promoters and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo All control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle and gonadotropic releasing hormone gene control region active in the hypothalamus, pyruvate kinase promoter, villin promoter, promoter of the fatty acid binding intestinal protein, promoter of the smooth muscle cell α-actin and the like. In addition, these expression sequences may be modified by addition of enhancer or regulatory sequences and the like.

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun or a DNA vector transporter (see, e.g., Wu et al., J Biol. Chem. 267:963 (1992); Wu et al., J Biol. Chem. 2(53:14621 (1988); and Hartmut et al., Canadian Patent Application No. 2,012,311, incorporated herein by reference in their entireties).

A polynucleotide according to the invention can also be introduced in vivo by lipofection. For the past decade, there has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker (Feigner et al., Proc. Natl. Acad. Sci. USA. 84:7413 (1987); Mackey et al., Proc. Natl Acad Sci. USA 55:8027 (1988); and Ulmer et al., Science 259: 1745 (1993), incorporated herein by reference in their entireties). The use of cationic lipids may promote encapsulation of negatively charged nucleic acids and also promote fusion with negatively charged cell membranes (Feigner et al., Science 337:387 (1989), incorporated herein by reference in its entirety). Particularly useful lipid compounds and compositions for transfer of nucleic acids are described in WO95/18863, WO96/17823 and U.S. Pat. No. 5,459,127, incorporated herein by reference in their entireties. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney and the brain. Lipids may be chemically coupled to other molecules for the purpose of targeting (Mackey et al. 1988, supra). Targeted peptides, e.g., hormones or neurotransmitters and proteins such as antibodies or non-peptide molecules could be coupled to liposomes chemically.

Other molecules are also useful for facilitating transfection of a nucleic acid in vivo, such as a cationic oligopeptide (e.g., WO95/21931, incorporated herein by reference in its entirety), peptides derived from DNA binding proteins (e.g., WO96/25508, incorporated herein by reference in its entirety) or a cationic polymer (e.g., WO95/21931, incorporated herein by reference in its entirety).

It is also possible to introduce a vector in vivo as a naked DNA plasmid (see U.S. Pat. Nos. 5,693,622, 5,589,466 and 5,580,859, incorporated herein by reference in their entireties). Receptor-mediated DNA delivery approaches can also be used (Curiel et al., Hum. Gene Ther. 3: 147 (1992); and Wu et al., J. Biol. Chem. 262:4429 (1987), incorporated herein by reference in their entireties).

The term “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

In addition, the recombinant vector comprising a polynucleotide according to the invention may include one or more origins for replication in the cellular hosts in which their amplification or their expression is sought, markers or selectable markers.

The term “selectable marker” refers to an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers and the like, wherein the effect is used to track the inheritance of a nucleic acid of interest and/or to identify a cell or organism that has inherited the nucleic acid of interest. Examples of selectable marker genes known and used in the art include: genes providing resistance to ampicillin, streptomycin, gentamycin, kanamycin, hygromycin, bialaphos herbicide, sulfonamide and the like; and genes that are used as phenotypic markers, i.e., anthocyanin regulatory genes, isopentanyl transferase gene and the like.

The term “reporter gene” refers to a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus) and the like. Selectable marker genes may also be considered reporter genes.

“Promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature or even comprise synthetic DNA segments. It is understood by those skilled in the art that different promoters may direct the expression of a gene in different tissues or cell types or at different stages of development or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

In any of the vectors of the present invention, the vector optionally comprises a promoter disclosed herein.

In any of the vectors of the present invention, the vector optionally comprises a tissue-specific promoter. In one embodiment, the tissue-specific promoter is a tissue specific promoter disclosed herein.

In some embodiments, the promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease SI), as well as protein binding domains (consensus sequences) responsible for the binding of RNA polymerase.

The term, “therapeutic switch promoter” (“TSP”) refers to a promoter that controls expression of a gene switch component. Gene switches and their various components are described in detail elsewhere herein. In certain embodiments a TSP is constitutive, i.e., continuously active. A constitutive TSP may be either constitutive-ubiquitous (i.e., generally functions, without the need for additional factors or regulators, in any tissue or cell) or constitutive-tissue or cell specific (i.e., generally functions, without the need for additional factors or regulators, in a specific tissue type or cell type). In certain embodiments a TSP of the invention is activated under conditions associated with a disease, disorder or condition. In certain embodiments of the invention where two or more TSPs are involved the promoters may be a combination of constitutive and activatable promoters. As used herein, a “promoter activated under conditions associated with a disease, disorder or condition” includes, without limitation, disease-specific promoters, promoters responsive to particular physiological, developmental, differentiation or pathological conditions, promoters responsive to specific biological molecules and promoters specific for a particular tissue or cell type associated with the disease, disorder or condition, e.g. tumor tissue or malignant cells. TSPs can comprise the sequence of naturally occurring promoters, modified sequences derived from naturally occurring promoters or synthetic sequences (e.g., insertion of a response element into a minimal promoter sequence to alter the responsiveness of the promoter).

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

The term, “transcriptional and translational control sequences” refer to DNA regulatory sequences, such as promoters, enhancers, terminators and the like, that provide for the expression of a coding sequence in a host cell. In eukaryotic cells, polyadenylation signals are control sequences.

The term “response element” refers to one or more cis-acting DNA elements which confer responsiveness on a promoter mediated through interaction with the DNA-binding domains of a transcription factor. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds, in the presence or absence of a ligand, to the DNA sequence of a response element to initiate or suppress transcription of downstream gene(s) under the regulation of this response element.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein refers to the transcription and stable accumulation of sense (mRNA) or antisense RNA derived from a nucleic acid or polynucleotide. Expression may also refer to translation of mRNA into a protein or polypeptide.

The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence and the like.

The term “gene switch” refers to the combination of a response element associated with a promoter and a ligand-dependent transcription factor-based system which, in the presence of one or more ligands, modulates the expression of a gene into which the response element and promoter are incorporated. The term “a polynucleotide encoding a gene switch” refers to the combination of a response element associated with a promoter and a polynucleotide encoding a ligand-dependent transcription factor-based system which, in the presence of one or more ligands, modulates the expression of a gene into which the response element and promoter are incorporated.

The therapeutic switch promoters of the invention may be any promoter that is useful for treating, ameliorating or preventing a specific disease, disorder or condition. Examples include, without limitation, promoters of genes that exhibit increased expression only during a specific disease, disorder or condition and promoters of genes that exhibit increased expression under specific cell conditions (e.g., proliferation, apoptosis, change in pH, oxidation state, oxygen level). In some embodiments where the gene switch comprises more than one transcription factor sequence, the specificity of the therapeutic methods can be increased by combining a disease- or condition-specific promoter with a tissue- or cell type-specific promoter to limit the tissues in which the therapeutic product is expressed. Thus, tissue- or cell type-specific promoters are encompassed within the definition of therapeutic switch promoter.

As an example of disease-specific promoters, useful promoters for treating cancer include the promoters of oncogenes. Examples of classes of oncogenes include, but are not limited to, growth factors, growth factor receptors, protein kinases, programmed cell death regulators and transcription factors. Specific examples of oncogenes include, but are not limited to, sis, erb B, erb B-2, ras, abl, myc and bcl-2 and TERT. Examples of other cancer-related genes include tumor associated antigen genes and other genes that are overexpressed in neoplastic cells (e.g., MAGE-1, carcinoembryonic antigen, tyrosinase, prostate specific antigen, prostate specific membrane antigen, p53, MUC-1, MUC-2, MUC-4, HER-2/neu, T/Tn, MART-1, gp100, GM2, Tn, sTn and Thompson-Friedenreich antigen (TF)).

### EXAMPLES

### Example 1—A Phase I Study of AD-RTS-HIL-12, an Inducible Adenoviral Vector Engineered to Express HIL-12 in the Presence of the Activator Ligand Veledimex in Subjects with Recurrent or Progressive Glioblastoma or Grade III Malignant Glioma

### Investigational Products

Adenovirus-RheoSwitch Therapeutic System®-human interleukin-12 (Ad-RTS-hIL-12) and veledimex (RTS activator ligand).

Ad-RTS-hIL-12 is a replication-incompetent adenoviral vector containing the hIL-12 gene under the control of the RTS inducible promoter turned on in the presence of the activator ligand, veledimex. Veledimex is a small molecule RTS-specific transcription factor that stabilizes the RTS promoter components and allows transcription of the hIL-12 target gene. Since target gene expression is dependent on the dose and frequency of veledimex administration, the expression of hIL-12 can be modulated (turned on and off) by the veledimex dose and schedule.

### Primary Objective

To determine the safety and tolerability of varying dose levels of intratumoral Ad-RTS-hIL-12 and oral veledimex doses in subjects with recurrent or progressive glioblastoma or Grade III malignant glioma.

### Secondary Objectives

To determine the veledimex maximum tolerated dose (MTD) when given with varying doses of intratumoral Ad-RTS-hIL-12
To determine the veledimex pharmacokinetic (PK) profile
To determine the veledimex concentration ratio between the brain tumor and blood
To evaluate cellular and humoral immune responses elicited by Ad-RTS-hIL-12 and veledimex
To determine investigator assessment of response, including tumor objective response rate (ORR) and progression-free survival (PFS)
To determine overall survival (OS)

### Study Design

This was a Phase I study (“Main Study”) of varying dose levels of Ad-RTS-hIL-12 administered by intratumoral injection and varying veledimex doses administered orally in subjects with recurrent or progressive glioblastoma or Grade III malignant glioma. This study investigated one intratumoral Ad-RTS-hIL-12 dose [2×10¹¹ viral particles (vp)] and four doses of veledimex (10 mg, 20 mg, 30 mg, and 40 mg) to determine the safe and tolerable dose based on the safety profiles observed in the presence of variable corticosteroid exposure.

This study was divided into three periods: the Screening Period, the Treatment Period and DLT evaluation period (Days 0-28), and the Follow-up Period. After the informed consent form (ICF) was signed, subjects entered the Screening Period to assess eligibility. Eligible subjects were stratified into one of two groups, according to clinical indication for tumor resection. Subjects who were scheduled for a standard of care craniotomy and tumor resection (Group 1) received one veledimex dose before the resection procedure. Samples (tumor, cerebrospinal fluid (CSF) (if available), and blood) were collected during the resection procedure to determine the veledimex concentration ratio between the tumor, the CSF (when available), and the blood. Ad-RTS-hIL-12 (2×10¹¹ vp) was administered by freehand injection into approximately two sites within the residual tumor for a total volume of 0.1 mL selected by the neurosurgeon. When available an intra-operative MRI was performed to guide the Ad-RTS-hIL-12 injection to areas of contrast-enhancing tumor tissue. After Ad-RTS-hIL-12 injection, subjects continued on oral veledimex for 14 days. Subjects not scheduled for tumor resection (Group 2) received Ad-RTS-hIL-12 (2×10¹¹ vp) by stereotactic injection and then continued on oral veledimex for 14 days.
Note: Subjects in Group 1 receive up to a total of 15 veledimex doses: one veledimex dose 3 (±2) hours before the craniotomy procedure (first dose prior to craniotomy will be a dose specific to the assigned cohort) and tumor resection (prior to Ad-RTS-hIL-12 administration), and up to 14 veledimex doses after Ad-RTS-hIL-12 administration. Subjects in Group 2 received up to a total of 14 veledimex doses after Ad-RTS-hIL-12 administration.

### Group 1: Subjects Scheduled for Craniotomy and Tumor Resection:

Subjects with a clinical indication for tumor resection received veledimex dose specific to the assigned cohort 3 (±2) hours before the craniotomy procedure, on an empty stomach (excluding other medications). At the time of tumor resection, brain tumor, CSF (if available), and blood samples were collected to determine the veledimex concentration ratio between brain tumor, CSF (if available), and blood.

For the four cohorts which were dosed (10 mg, 20 mg, 30 mg, and 40 mg cohorts) immediately after tumor resection, Ad-RTS-hIL-12 2×10¹¹ vp was administered by freehand injection into approximately two sites within the residual tumor for a total volume of 0.1 mL. The total amount delivered to each site was recorded in the CRF. In the event that less than the planned total injected volume was administered, the reason was provided. The day of Ad-RTS-hIL-12 administration was designated as Day 0.

After the Ad-RTS-hIL-12 injection, veledimex was administered orally for 14 days. The first postresection veledimex dose was given on Day 1, preferably with food. Subsequent veledimex doses were taken once daily, in the morning and within approximately 30 minutes of a regular meal.

### Group 2: Subjects NOT Undergoing Tumor Resection:

Subjects who did not undergo tumor resection received Ad-RTS-hIL-12 by standard stereotactic surgery on Day 0. Ad-RTS-hIL-12 (2×10¹¹ vp) was administered by stereotactic injection. The day of Ad-RTS-hIL-12 administration was designated as Day 0 and it was delivered into approximately two intratumoral sites for a total volume of 0.1 mL. The total amount delivered to each site was recorded in the CRF. In the event that less than the planned total injected volume was administered, the reason was provided. Care was taken to avoid intraventricular or basal cisternal injection or other critical locations.

After the Ad-RTS-hIL-12 injection, veledimex (20 mg) was administered orally for 14 days. The first veledimex dose was given on Day 1, preferably with food. Subsequent veledimex doses were taken once daily, in the morning and within approximately 30 minutes of a regular meal.

Subject enrollment into Group 2 started no earlier than after two subjects had completed 28 days in Group 1, Cohort 1. Enrollment into Group 2 cohorts was otherwise independent of enrollment into Group 1 cohorts.

This study explored four veledimex dose cohorts: 10 mg, 20 mg, 30 mg, and 40 mg. Subject enrollment and veledimex dose escalation proceeded according to a standard 3+3 design modified to independently evaluate two stratified subject groups that may exhibit different safety and tolerability profiles. In each cohort, the first subject was monitored for the 14 days of treatment and observed for an additional 7 days post the last veledimex dose before the second and third subjects were enrolled in that same cohort. The dose-limiting toxicity (DLT) evaluation period was defined as 28 days post Ad-RTS-hIL-12 injection (Day 0-Day 28). Determination of safety and recommendation to dose escalate occurred after all dosed subjects in a cohort had been evaluated for at least 28 days post Ad-RTS-hIL-12 injection. The details on the dose escalation schedule are provided in Table 1 and the dose escalation decision rules provided in Table 2.

TABLE 1
Veledimex Dose-Escalation Cohorts Post Ad-RTS-hIL-12 Injection

| | | Veledimex^{b} |
|---|---|---|
| | Ad-RTS-hIL-12^{a} | Group 1 (Days 0 through 14) |
| | (Day 0) | Group 2 (Days 1 through 14) |
| Cohort | Dose(vp) | Total Daily Dose (mg) |
| 1 | 2 × 10¹¹ | 20 |
| 2 | 2 × 10¹¹ | 40 |
| 3 | 2 × 10¹¹ | 30 |
| 4 | 2 × 10¹¹ | 10 |

vp = viral particle
^{a}Intratumoral Injection
^{b}Dose levels may have been modified based on additional clinical and nonclinical data

TABLE 2
Dose Escalation Decision Rules

| Subjects with DLTs | |
|---|---|
| (Any Dose Level) | Escalation Decision Rule |
| No subject experience | Enroll the next higher dose level cohort (after DSMB recommendation). |
| a DLT | |
| 1 subject experiences a | Enroll at least three more subjects in the same group at this dose level |
| DLT | cohort. |
| | If zero or one of the three additional subjects experience a DLT, proceed |
| | to the next planned dose level cohort (after DSMB recommendation). |
| 2 or more subjects | If two subjects experience a DLT (two DLTs in Group 1 or two DLTs in |
| experience a DLT | Group 2), then STOP dose escalation in the cohort experiencing the DLTs |
| | and do the following: |
| | The SRC will conduct a safety review to evaluate safety and |
| | determine whether the cohort experiencing the two DLTs has met |
| | the definition of the MTD Based upon this review, the SRC will |
| | make a recommendation that the cohort experiencing the ≥two |
| | DLTs either continue at the existing dose, at a lower dose level or |
| | other measures to be undertaken including discontinuation of |
| | treatment. After completion of a cohort, if it has been determined |
| | that the MTD has not been reached, escalation to the next cohort |
| | will proceed once recommended by the SRC and authorized by |
| | the DSMB. If it has been determined that escalation should not |
| | proceed, dose de-escalation will be undertaken. |

### Concomitant Therapy

Information on concomitant medications, including all medications, blood products, vitamins, and other supplements, was collected through the Screening, Treatment, and the Initial Follow-up Period of this study.

Subjects experiencing brain tumor-related symptoms or edema were treated with corticosteroids as per standard practice. The treating physician put into consideration the minimum starting steroid dose for study subjects, if it was determined to be safe and appropriate for that individual patient. For study subjects who required a higher starting steroid dose, efforts were made to taper steroids to the lowest amount that controlled the subjects' symptoms, as determined to be safe and appropriate by the treating physician

### Permitted Medications

Subjects could receive standard treatments, including palliative and supportive care for any illness or symptom management during study treatment, including:

Corticosteroids were permitted for brain tumor-related symptoms. The treating physician was to consider the minimum steroid dose for study subjects, if determined to be safe and appropriate for that individual patient. For study subjects who required a higher steroid dose, efforts were made to taper steroids to the lowest amount that controlled the subjects symptoms, as determined to be safe and appropriate by the treating physician.

Antidiarrheal therapy was permitted for study drug-induced diarrhea.

Anti-emetics were permitted for study drug-induced nausea and vomiting.

NOTE: Care was taken when prescribing medications that are classified as CYP450 3A4 inducers, inhibitors, or substrates due to potential interactions with the study drug. In the event that one was prescribed, consultation with the Medical Monitor was advised. All medications were recorded in the case report form as indicated in the completion guidelines.

### Prohibited Medications

The following medications were prohibited during the study:

Any other investigational agent or anticancer therapy (chemotherapy, radiotherapy, etc.) while receiving study treatment.

Palliative radiotherapy was not permitted while on study.

NOTE: Care was given when prescribing medications that are classified as CYP450 3A4 inducers, inhibitors, or substrates due to potential interactions with the study drug. In the event that one was prescribed, consultation with the Medical Monitor was advised. All medications were recorded in the case report form as indicated in the completion guidelines.

### Study Procedures

### Schedule of Procedures and Observations

Refer to Table 4. Screening assessments were performed within 28 days prior to the Ad-RTS-hIL-12 injection. Any screening tests, exams, or procedures outside of this range could be repeated at the investigator's discretion. All study visits were completed as described in the protocol while subjects were taking veledimex capsules. Follow-up assessments were allowed a window of ±7 days.

### Study Tests, Exams, and Procedures

### Demographics, Medical and Cancer History, and Concomitant Medications

Each subject's complete medical history was documented during screening, including demographic information, relevant medical history, current primary cancer diagnosis, and prior cancer treatments (chemo- and immunotherapies, radiation therapy, surgeries, and any associated residual toxicities). In addition, concomitant medications, including blood products, vitamins, and other supplements received during the screening period (28 days) prior to initiating study treatment were recorded. Concomitant medications continued to be collected through the Initial Follow-Up period (e.g. Day 56).

### Physical Examinations

A complete physical examination was conducted, including a neurological examination.

### Vital Signs, Height, and Weight

Vital signs include blood pressure, pulse rate, temperature, and respiration rate. Subject's blood pressure was monitored closely, with hydration as needed to prevent hypotension for 72 hours after administration of Ad-RTS-hIL-12. Assessment of vital signs was required prior to injection of Ad-RTS-hIL-12, and prior to veledimex dosing. Height and weight were measured and recorded according to Schedule of Study Procedures (see Table 4).

### Karnofsky Performance Status

The Karnofsky Performance Status measures the ability of cancer subjects to perform ordinary tasks. Scores range from 0 to 100 with a higher score meaning that the patient is better able to carry out daily activities. The Karnofsky Performance Status is used to determine a patient's prognosis, to measure changes in a patient's ability to function, or to decide if a patient could be included in a clinical trial. Subjects had to have a Karnofsky Performance Status score of ≥70 at the Screening Visit to be included in the study (Table 5. Karnofsky Performance Status).

### Pregnancy Testing

Females of childbearing potential had a serum pregnancy test at the Screening Visit and a urine or serum pregnancy test on Day 0, with a negative pregnancy outcome prior to study drug initiation.

### Tumor Response Assessments

### Tumor Response

The secondary time-to event endpoints of this study include Investigator assessment of ORR, PFS and OS.

Tumor response was evaluated radiographically using MRI scans to determine tumor response and to assess the time of objective disease progression (estimate of PFS). A baseline MRI was performed within 72 hours of Ad RTS hIL 12 administration (Day 2). The Ad RTS hIL 12 injected lesion and/or other measurable brain lesions were measured according to the RANO/iRANO criteria guidelines. MRI scans were collected and stored at the study site and each subject was evaluated for response by the study investigator. Subjects were imaged throughout the study using the same method(s) as were used for the screening and baseline MRIs. Independent tumor response assessments, as well as posttreatment tumor biopsies, occurred as available and at the discretion of the investigator. A repeat scan to confirm progression was completed at 4 weeks (per RANO) and again at 12 weeks (per iRANO) after first documentation of progression. Consideration was given to performing a diagnostic brain biopsy, which should be performed in accordance with the current iRANO guidelines.

Response was defined by radiographic and clinical criteria. Complete response (CR) or partial response (PR) was first assessed by radiographic changes that indicate a reduction of bidimensional tumor size as per RANO/iRANO criteria. In addition, changes in neurologic function and steroid use were considered to determine stable disease (SD).

Tumor response assessments occurred at 4 weeks (Day 28±7 days), 8 weeks (Day 56±7 days), and every 8 weeks thereafter for all subjects, including those who may have experienced a dose delay or missed a dose, until the occurrence of confirmed tumor progression, initiation of alternative therapy, or one year, whichever occurred first.

### Tumor Response Evaluation and Pseudo Progression

The interpretation of MRI findings in subjects with treated brain tumors has an inherent uncertainty that stems from the pseudo progression phenomena. Pseudo progression is a term used to describe the appearance of radiographic disease progression due to increase contrast enhancement on MRI without true tumor progression. The increase in contrast enhancement can be influenced by several parameters including differences in radiologic technique, the amount of contrast agent used, the timing of the contrast agent administration relative to the imaging, postsurgical changes, infarction, treated related inflammation, seizure activity, sub-acute radiation effects, radiation necrosis, and corticosteroid use. Consideration of these factors by experts and clinical experience is likely to identify these subjects. In this study, pseudo progression was unlikely to impact the duration of therapy since the veledimex treatment period lasts 14 days and the first tumor assessment MRI was done on Day 28 (±7 days). Imaging assessments will be performed using RANO/iRANO criteria.

### Monitoring of Adverse Events

Monitoring and recording of AEs and serious adverse events (SAEs) was conducted throughout the study. Adverse events and SAEs that occurred following the signing of the ICF through the Initial Follow-up Period (e.g. Day 56) were recorded on the AE CRF.

Proper hydration is critical. Subjects were instructed repeatedly to maintain adequate oral hydration on and between veledimex doses; study sites closely monitored subjects' hydration status. Blood pressure was monitored regularly.

Administration of prophylactic antipyretics was strongly recommended during the first week after Ad-RTS-hIL-12 injection.

### Clinical Laboratory Assessments

The hematology panel comprised of a complete blood count (CBC), including white blood cell (WBC) count with differential, red blood cell (RBC) count, hematocrit, hemoglobin, red blood cell indices, mean corpuscular volume (MCV), and platelet count.

The serum chemistry panel comprised of the following parameters: AST, ALT, lactate dehydrogenase (LDH), alkaline phosphatase (ALP), creatinine, total bilirubin, total protein, albumin, amylase, blood urea nitrogen (BUN), glucose, sodium, potassium, chloride, calcium, phosphorus, and bicarbonate.

The coagulation panel included activated partial thromboplastin time (aPTT) and INR.

The urinalysis panel (dipstick) included appearance, pH, specific gravity, glucose, protein/albumin, presence of blood, ketones, bilirubin, nitrates, and leukocyte esterase. In addition, a microscopic exam for casts, crystals, and cells were done if clinically indicated.

### Number of Subjects

39 subjects were enrolled.

### Study Population

The eligible study population included adult subjects with recurrent or progressive glioblastoma or Grade III malignant glioma (anaplastic astrocytoma, anaplastic oligodendroglioma, and anaplastic oligoastrocytoma) for which there is no alternative curative therapy.

### Inclusion Criteria:

1. Male or female subject ≥18 and ≤75 years of age
2. Provision of written informed consent for tumor resection, stereotactic surgery, tumor biopsy, samples collection, and treatment with investigational products prior to undergoing any study-specific procedures
3. Histologically confirmed supratentorial glioblastoma or other World Health Organization (WHO) Grade III or IV malignant glioma from archival tissue
4. Evidence of tumor recurrence/progression by MRI according to response assessment in neuro-oncology (RANO) criteria after standard initial therapy
5. Previous standard of care antitumor treatment including surgery and/or biopsy and chemoradiation. At the time of registration, subjects had to have recovered from the toxic effects of previous treatments as determined by the treating physician. The washout periods from prior therapies were intended as follows: (windows other than what is listed below were allowed only after consultation with the Medical Monitor)
   a. Nitrosoureas: 6 weeks
   b. Other cytotoxic agents: 4 weeks
   c. Antiangiogenic agents, including bevacizumab: 4 weeks
   d. Targeted agents, including small-molecule tyrosine kinase inhibitors: 2 weeks
   e. Experimental immunotherapies (e.g., PD-1, CTLA-4): 3 months
   f. Vaccine-based therapy: 3 months
6. Able to undergo standard MRI scans with contrast agent before enrollment and after treatment
7. Karnofsky Performance Status ≥70
8. Adequate bone marrow reserves and liver and kidney function, as assessed by the following laboratory requirements:
   a. Hemoglobin ≥9 g/L
   b. Lymphocytes >500/mm³
   c. Absolute neutrophil count ≥1500/mm³
   d. Platelets ≥100,000/mm³
   e. Serum creatinine ≤1.5× upper limit of normal (ULN)
   f. Aspartate transaminase (AST) and alanine transaminase (ALT)≤2.5×ULN. For subjects with documented liver metastases, ALT and AST≤5×ULN
   g. Total bilirubin <1.5×ULN
   h. International normalized ratio (INR) and activated partial thromboplastin time within normal institutional limits
9. Male and female subjects had to agree to use a highly reliable method of birth control (expected failure rate less than 5% per year) from the Screening Visit through 28 days after the last dose of study drug. Women of childbearing potential (perimenopausal women must be amenorrheic for at least 12 months to be considered of non-childbearing potential) had to have a negative pregnancy test at screening.

### Exclusion Criteria:

1. Radiotherapy treatment within 4 weeks or less prior to starting first veledimex dose
2. Subjects with clinically significant increased intracranial pressure (e.g., impending herniation or requirement for immediate palliative treatment) or uncontrolled seizures
3. Known immunosuppressive disease, autoimmune conditions, and/or chronic viral infections [e.g., human immunodeficiency virus (HIV), hepatitis]
4. Use of systemic antibacterial, antifungal, or antiviral medications for the treatment of acute clinically significant infection within 2 weeks of first veledimex dose. Concomitant therapy for chronic infections was not allowed. Subjects had to be afebrile prior to Ad-RTS-hIL-12 injection; only prophylactic antibiotic use was allowed perioperatively.
5. Use of enzyme-inducing antiepileptic drugs (EIAED) within 7 days prior to the first dose of study drug. Note: Levetiracetam (Keppra®) is not an EIAED and was not allowed
6. Other concurrent clinically active malignant disease, requiring treatment, with the exception of non-melanoma cancers of the skin or carcinoma in situ of the cervix or nonmetastatic prostate cancer
7. Nursing or pregnant females
8. Prior exposure to veledimex
9. Use of medications that induce, inhibit, or are substrates of CYP450 3A4 within 7 days prior to veledimex dosing without consultation with the Medical Monitor
10. Presence of any contraindication for a neurosurgical procedure
11. Unstable or clinically significant concurrent medical condition that would, in the opinion of the investigator or medical monitor, jeopardize the safety of a subject and/or their compliance with the protocol. Examples include, but are not limited to, unstable angina, congestive heart failure, myocardial infarction within 2 months of screening, ongoing maintenance therapy for life-threatening ventricular arrhythmia or uncontrolled asthma.

### Study Assessments and Criteria for Evaluation

### Immune Response Assessments:

Immunological and biological markers, such as levels of IL-12, interferon-gamma (IFN-γ), interferon gamma-induced protein 10 (IP-10), IL-2, IL-10, and neutralizing antibodies to viral components or hIL-12 were assessed in pre- and posttreatment serum samples.

Immune cell population markers such as cluster of differentiation (CD) antigens CD3, CD4, CD8, CD25, and FOX-P3, CD56, CD45RO, and human leukocyte antigen allele status were assessed as scheduled in the Schedule of Study Procedures.

### Pharmacokinetic Evaluations:

Veledimex PK parameters were evaluated at each dose level in the dose escalation and any proposed expansion cohorts for Group 1 and Group 2 subjects. PK sampling times are outlined as follows below.

Veledimex PK assessment was done for all subjects during study treatment. Whole blood samples were collected at the time points specified in the Schedule of Veledimex Pharmacokinetic Sampling Times (Table 3). Veledimex plasma concentrations were determined using a fully validated liquid chromatography-mass spectrometry assay.

TABLE 3
Schedule of Veledimex Pharmacokinetic Sampling Times

| Sample | Day 0 | Day 1 | Day 2 | Day 3 | Day 7 | Day 14 | Day 15 |
|---|---|---|---|---|---|---|---|
| 1 | During | Predose^{a} | Predose^{a} | Predose^{a} | Predose^{a} | Predose^{a} | Scheduled |
| | Resection | | | | | | Visit^{b} |
| 2 | — | 3 to 5 hours | — | — | — | 3 to 5 hours | — |
| | | after dose | | | | after dose | |

^{a}≤30 minutes prior to veledimex dose
^{b}24 hours post last dose (Day 14)

### Study Duration

The duration of this study from the time of initiating subject screening until the completion of survival follow was anticipated to be approximately 48 months, including 24 months for enrollment and 24 months of follow-up.

The start of study was defined as the date when the first subject is consented into the study and the study stop date was the date of database lock.

TABLE 4
Schedule of Study Procedures

| | | | | | | | | | | | | | Long Term |
|---|---|---|---|---|---|---|---|---|---|---|---|---|---|
| | Screening | | | | | | | | | Initial Follow-up | | | Follow-up |
| | Period | Treatment Period | | | | | | | | Period | | | Period |
| | Day | Day | Day | Day | Day | Days | Day | Days | Day | Day | Day | Day | Every |
| Activity | −28 to −1 | 0 | 1 | 2 | 3 | 4-6 | 7 | 8-13 | 14 | 15 | 28 ± 7 | 56 ± 7 | 8 weeks |
| Clinical Assessments | | | | | | | | | | | | | |
| Informed Consent | X | | | | | | | | | | | | |
| Medical/Cancer | X | | | | | | | | | | | | |
| History^{a,b} | | | | | | | | | | | | | |
| Physical Exam^{c}, | X | X | X | X | X | | X | | X | | X | | |
| including targeted | | | | | | | | | | | | | |
| neurological exam | | | | | | | | | | | | | |
| Karnofsky PS^{d} | X | X | | | | | | | X | | X | | |
| Height (only at | X | | | | | | X | | X | | X | | |
| Screen) and | | | | | | | | | | | | | |
| Weight | | | | | | | | | | | | | |
| Vital Signs^{e} | X | X | X | X | X | | X | | X | | X | | |
| Adverse Events^{f} | X | → | | | | | | | | | | X | |
| Concomitant | X | → | | | | | | | | | | X | X^{g} |
| Medications^{b,f} | | | | | | | | | | | | | |
| Survival Status^{g} | X | → | | | | | | | | | | | |
| Clinical Laboratory^{a} | | | | | | | | | | | | | |
| Pregnancy Test^{h} | X | X | | | | | | | | | | | |
| Hematology | X | X | | X | X | | X | | X | | X | | |
| Panelⁱ | | | | | | | | | | | | | |
| Coagulation | X | X | | | X | | X | | X | | | | |
| Panel^{j} | | | | | | | | | | | | | |
| Serum Chemistry | X | X | | X | X | | X | | X | | X | | |
| Panel^{k} | | | | | | | | | | | | | |
| Urinalysis Panel^{l} | X | X | | | | | | | X | | | | |
| ECG^{m} | X | X | | | X | | | | X | | | | |
| Registrationⁿ | X | | | | | | | | | | | | |
| Study Drug Administration | | | | | | | | | | | | | |
| Ad-RTS-hIL-12 | | X^{o,p} | | | | | | | | | | | |
| Veledimex Dose | | X | X^{p,q} | X | X | X | X | X | X^{r} | | | | |
| Group 1 | | | | | | | | | | | | | |
| Veledimex Dose | | | X^{p,q} | X | X | X | X | X | X^{r} | | | | |
| Group 2 | | | | | | | | | | | | | |
| Veledimex Dose | | | X | X | X | X | X | X | X | | | | |
| Compliance/Subject | | | | | | | | | | | | | |
| Diary^{r} | | | | | | | | | | | | | |
| PK/PD/Immune Assessments | | | | | | | | | | | | | |
| Viral Shedding | X | | | | X | | | | | | | | |
| Assessment | | | | | | | | | | | | | |
| Veledimex PK | | X^{t} | X | X | X | | X | | X | X | | | |
| blood sample^{s, u} | | | | | | | | | | | | | |
| Serum Cytokine | | X^{t} | | | X | | X | | X | | X | | |
| profile^{u} | | | | | | | | | | | | | |
| Immune Function | | X | | | | | X | | X | | X | | |
| blood sample^{u} | | | | | | | | | | | | | |
| MRI Scans^{v,w} | X^{v,w} | | | X^{v,w,x} | | | | | X^{v} | | X^{w} | X^{w} | X^{w} |
| Tumor Sample^{u} | | X | | | | | | | | | | | |
| (Group 1 only) | | | | | | | | | | | | | |
| CSF Sample^{y} | | X | | | | | | | | | | | |
| (Group 1 only) | | | | | | | | | | | | | |

^{a}Medical history includes demographic information, medical and surgical history. Cancer history includes current cancer diagnosis, prior treatment [regimen(s), doses, start and stop dates and any associated residual toxicity], and best response for each regimen.
^{b}Medications received in the period preceding consent (~28 days) in addition to those ongoing at screening were captured in the CRF.
^{c}A complete physical examination including a neurological exam and mental status was required at baseline. Targeted neurological exams thereafter.
^{d}See Table 5. Karnofsky Performance Status
^{e}Blood pressure, pulse, temperature, and respiration were recorded. Blood pressure was monitored closely, with hydration as needed to prevent hypotension after veledimex administration. Subjects were instructed to maintain adequate oral hydration on and between veledimex dosing days; sites closely monitored subjects' hydration status.
^{f}Monitoring and recording of concomitant medications and adverse events (AEs) and serious adverse events (SAEs) were conducted throughout the study. Concomitant medications given and AEs/SAEs that occur following signed informed consent form (ICF) through the initial Follow-up Period (e.g. Day 56 visit) were recorded in the CRF. AEs that were ongoing at the end of the Initial Follow-up Period and considered drug related were followed until resolution or no resolution was expected.
^{g}Patients were followed to document start of a new anticancer therapies and survival status for 2 years following administration of Ad-RTS-hIL-12.
^{h}Females of childbearing potential had a serum pregnancy test at the Screening Visit and a urine or serum pregnancy test on Day 0, with a negative pregnancy outcome required prior to first dose of study drug (either veledimex (Group 1) or injection of Ad-RTS-hIL-12 (Group 2)).
ⁱHematology Panel: complete blood count, white blood count with differential, red blood cell count, hematocrit, hemoglobin, red blood cell indices, mean corpuscular volume, and platelet count.
^{j}Coagulation Panel: activated partial thromboplastin time, international normalized ratio, erythrocyte sedimentation rate and C-reactive protein.
^{k}Serum Chemistry Panel: aspartate transaminase, alanine transaminase, lactate dehydrogenase, alkaline phosphatase, creatinine, total bilirubin, total protein, albumin, amylase, blood urea nitrogen, glucose, sodium, potassium, chloride, calcium, phosphorus and bicarbonate.
^{l}Urinalysis Panel (dipstick): appearance, pH, specific gravity, glucose, protein/albumin, blood, ketones, bilirubin, nitrates, and leukocyte esterase. In addition, a microscopic exam for casts, crystals and cells were done if clinically indicated.
^{m}Standard 12-lead ECG; single measurement at each time point
ⁿCentralized registration of eligible subjects were completed prior to first dose of study drug (either veledimex (Group 1) or injection of Ad-RTS-hIL-12 (Group 2)), according to a process defined by the sponsor.
^{o}Ad-RTS-hIL-12 intratumoral injection were administered by freehand injection for Group 1 subjects and intracranial stereotactic injection for Group 2 subjects. Subjects were instructed to maintain adequate oral hydration during the Treatment Period; sites closely monitored subjects' hydration status. Because of the potential for toxicity (e.g., fevers, chills, fatigue and dehydration), administration of prophylactic antipyretics was recommended after injection of Ad-RTS-hIL-12.
^{p}Each subject was carefully monitored for any local reactions and/or hypersensitivity reactions following the Ad-RTS-hIL-12 injection and veledimex administration. Subjects were instructed to call the clinical site if headache, hemiparesis, seizure or other local reactions develop anytime and especially between study visits.
^{q}The first postresection veledimex dose was given on the next day, designated as Day 1, preferably with food. Subsequent veledimex doses were taken once daily, in the morning and within 30 minutes of a regular meal.
^{r}Study sites determined compliance of veledimex dosing. Subjects were instructed to document veledimex dosing compliance in a subject diary, including the time subject took the once daily dose, the time subject ate breakfast, the number of capsules taken, whether subject missed any veledimex doses and the study day and reason for any missed doses. Study drug container(s) with any remaining capsules were returned to the study staff, so that staff could properly assess dose compliance.
^{s}The PK sampling schedule is provided in Table 3.
^{t}Day 0, PK, and cytokine blood samples were obtained prior to Ad-RTS-hIL-12 injection.
^{u}Additional ad hoc samples, including blood, tissue, excretions, etc., could be requested to evaluate safety. In the event of a drug-related AE, if possible, an unscheduled visit kit was obtained for cytokines and immunological markers for CSF evaluation, if applicable.
^{v}Appropriate cancer staging procedures were performed during screening. All imaging was of diagnostic quality. The brain was imaged using the same method(s) used throughout the study. Measurable target lesions were selected and measured per RANO/iRANO guidelines. A repeat scan to confirm progression was completed at 4 weeks (per RANO) and preferably again at 12 weeks (per iRANO) after first documentation of progression. Additional tumor response assessments as well as a posttreatment diagnostic brain biopsy were performed at the discretion of the investigator as part of providing standard of care treatment in accordance with current iRANO guidelines.
^{w}The Day 28 (±7 days) and Day 56 (±7 days) MRI scans were required for all subjects, including those with unconfirmed disease progression, to ensure that more slowly declining tumor burden in response to therapy was noted. For 2 years, subjects without confirmed disease progression continued to have tumor assessments performed every 8 weeks as per standard practice until disease progression had been identified (first documentation) and confirmed (12 weeks after first documentation). MRI scans had to be available for collection upon sponsor request.
^{x}The MRI scan designated on Day 2 was taken within 72 hours of Ad-RTS-hIL-12 administration and was considered the baseline scan for tumor response assessments.
^{y}Additional tumor, CSF (if available) and blood samples were collected, if available, as part of standard of care procedures.

TABLE 5
Karnofsky Performance Status
Karnofsky Performance Status Scale
Definitions Rating (%) Criteria

| | | |
|---|---|---|
| Able to carry on normal activity | 100 | Normal no complaints; no evidence of |
| and work; no special care needed. | | disease |
| | 90 | Able to carry on normal activity; minor signs |
| | | of symptoms of disease |
| | 80 | Normal activity with efforts; some signs of |
| | | symptoms of disease |
| Unable to work; able to live at | 70 | Cares for self; unable to carry on normal |
| home and care for most personal | | activity or to do active work |
| needs; varying amount of | 60 | Requires occasional assistance, but is able to |
| assistance needed. | | care for most of his personal needs |
| | 50 | Requires considerable assistance and frequent |
| | | medical care |
| Unable to care for self; requires | 40 | Disabled; requires special care and assistance |
| equivalent of institutional or | 30 | Severely disabled; hospital admission is |
| hospital care; diseases may be | | indicated although death not imminent |
| progressing rapidly. | 20 | Very sick; hospital admission necessary; |
| | | active support treatment necessary |
| | 10 | Moribund; fatal processes progressing rapidly |
| | 0 | Dead |

Oxford Textbook of Palliative Medicine, Oxford University Press. 1993, 109.

### Example 2—Substudy: Evaluation of AD-RTS-HIL-12+ Veledimex in Subjects with Recurrent or Progressive Glioblastoma

### Introduction

This protocol describes a substudy (“Substudy”) to the Phase I protocol of Example 1 examining the treatment of subjects with recurrent or progressive Grade IV glioblastoma with adenovirus RheoSwitch Therapeutic System® human interleukin 12 (Ad RTS hIL 12)+veledimex (RTS® activator ligand). In this substudy, subjects who were candidates for resection received Ad RTS hIL 12 (2×10¹¹ viral particles [vp]) and 20 mg of veledimex administered orally (PO).

Preliminary data from the main study of patients treated with Ad-RTS-hIL-12 and veledimex following tumor resection who had no prior exposure to bevacizumab and had lower concurrent exposure to corticosteroids showed an enhanced survival benefit. In order to minimize the variability of the study population, prior use of bevacizumab as primary treatment and use of corticosteroids in the 4 weeks prior to study entry was excluded in this substudy. The 20 mg dose of veledimex chosen for this substudy to further expand as preliminary analysis showed the best survival and tolerance of the various doses of veledimex studied in the main study of Example 1 (10, 20, 30 and 40 mg).

In summary this substudy further explored the safety, and potential efficacy, of Ad-RTS-hIL-12+veledimex in patients, planned to undergo resection, who had not received prior bevacizumab for the treatment of their disease and had not been on corticosteroids for at least for 4 weeks prior to receiving Ad-RTS-hIL-12+veledimex.

### Substudy Objectives

### Primary Substudy Objective

To determine the safety and tolerability of Ad-RTS-hIL-12 and veledimex (RTS activator ligand) in subjects with recurrent or progressive glioblastoma.

### Secondary Substudy Objectives

To determine the overall survival (OS) of Ad-RTS-hIL-12+veledimex
To determine the veledimex pharmacokinetic (PK) profile
To determine the veledimex concentration ratio between the brain tumor and blood
To determine the Investigator's assessment of response, including tumor objective response rate (ORR), progression free survival (PFS), and rate of pseudo progression (PSP)
To evaluate cellular and humoral immune responses elicited by the combination treatment

### Subject Selection

Only subjects eligible to enter the current protocol were included in this study. For all subjects who consented to participating in this substudy, all screening assessments occurred as in the main protocol (see Example 1 above).

The eligible study population included adult subjects with recurrent or progressive Grade IV glioblastoma (herein after referred to as glioblastoma) for which there is no alternative curative therapy who had not previously been treated with bevacizumab for their disease (short use (<4 doses) of bevacizumab for controlling edema is allowed) and had not received corticosteroids during the previous 4 weeks. Note: Subjects with Grade III malignant glioma were not eligible to participate in this substudy.

### Inclusion Criteria as in the Main Protocol of Example 1 (Differences are Underlined)

1. Male or female subject ≥18 and ≤75 years of age.
2. Provision of written informed consent for tumor resection, stereotactic surgery, tumor biopsy, samples collection, and treatment with investigational products prior to undergoing any study specific procedures.
3. Histologically confirmed supratentorial glioblastoma.
4. Evidence of tumor recurrence/progression by magnetic resonance imaging (MRI) according to response assessment in neuro-oncology (RANO) criteria after standard initial therapy.
5. Previous standard-of-care antitumor treatment including surgery and/or biopsy and chemoradiation. At the time of registration, subjects must have recovered from the toxic effects of previous treatments as determined by the treating physician. The washout periods from prior therapies are intended as follows: (windows other than what is listed below should be allowed only after consultation with the Medical Monitor).
   a. Nitrosoureas: 6 weeks
   b. Other cytotoxic agents: 4 weeks
   c. Antiangiogenic agents: 4 weeks (NOTE: short use (<4 doses) of bevacizumab for controlling edema is allowed)
   d. Targeted agents, including small molecule tyrosine kinase inhibitors: 2 weeks
   e. Experimental immunotherapies (e.g., PD-1, CTLA-4): 3 months
   f. Vaccine-based therapy: 3 months
6. Able to undergo standard MRI scans with contrast agent before enrollment and after treatment.
7. Karnofsky Performance Status ≥70.
8. Adequate bone marrow reserves and liver and kidney function, as assessed by the following laboratory requirements:
   a. Hemoglobin ≥9 g/L
   b. Lymphocytes >500/mm³
   c. Absolute neutrophil count ≥1500/mm³
   d. Platelets ≥100,000/mm³
   e. Serum creatinine ≤1.5× upper limit of normal (ULN)
   f. Aspartate transaminase (AST) and alanine transaminase (ALT)≤2.5×ULN. For subjects with documented liver metastases, ALT and AST≤5×ULN
   g. Total bilirubin <1.5×ULN
   h. International normalized ratio (INR) and aPTT or partial thromboplastin time (PTT) within normal institutional limits
9. Male and female subjects had to agree to use a highly reliable method of birth control (expected failure rate <5% per year) from the Screening Visit through 28 days after the last dose of study drug. Women of childbearing potential (perimenopausal women had to be amenorrheic for at least 12 months to be considered of non-childbearing potential) with a negative pregnancy test at screening.

### Exclusion Criteria (as in the Main Protocol of Example 1)

1. Radiotherapy treatment within 4 weeks or less prior to veledimex dosing.
2. Subjects with clinically significant increased intracranial pressure (e.g., impending herniation or requirement for immediate palliative treatment) or uncontrolled seizures.
3. Known immunosuppressive disease, or autoimmune conditions, and/or chronic viral infections (e.g., human immunodeficiency virus [HIV], hepatitis).
4. Use of systemic antibacterial, antifungal, or antiviral medications for the treatment of acute clinically significant infection within 2 weeks of first veledimex dose. Concomitant therapy for chronic infections was not allowed. Subjects had to be afebrile prior to Ad-RTS-hIL-12 injection; only prophylactic antibiotic use was allowed perioperatively.
5. Use of enzyme inducing antiepileptic drugs (EIAED) within 7 days prior to the first dose of study drug. Note: Levetiracetam (Keppra®) is not an EIAED and was allowed.
6. Other concurrent clinically active malignant disease, requiring treatment, with the exception of non-melanoma cancers of the skin or carcinoma in situ of the cervix or nonmetastatic prostate cancer.
7. Nursing or pregnant females.
8. Prior exposure to veledimex.
9. Use of medications that induce, inhibit, or are substrates of cytochrome p450 (CYP450) 3A4 within 7 days prior to veledimex dosing without consultation with the Medical Monitor.
10. Presence of any contraindication for a neurosurgical procedure.
11. Unstable or clinically significant concurrent medical condition that would, in the opinion of the Investigator or Medical Monitor, jeopardize the safety of a subject and/or their compliance with the protocol. Examples include, but are not limited to: unstable angina, congestive heart failure, myocardial infarction within 2 months of screening, ongoing maintenance therapy for life-threatening ventricular arrhythmia or uncontrolled asthma.

### Exclusion Criteria Specific to the Substudy:

1. Previous treatment with bevacizumab for their disease (NOTE: short use (<4 doses) of bevacizumab for controlling edema was allowed)
2. Subjects receiving systemic corticosteroids during the previous 4 weeks.
   Withdrawal of Subjects from Study Treatment and/or Study and Replacement of Subjects

Subjects followed the withdrawal criteria as specified in the main protocol of Example 1. All dosed subjects were included in the overall safety assessment.

### Schedule of Events

The schedule of assessments follow the main protocol of Example 1.

At all visits, concomitant medications, adverse events, and survival status were documented: Concomitant medications were monitored and recorded throughout the study. Medications received in the period preceding consent (˜28 days), in addition to those ongoing at screening, were captured in the CRF. Non-serious events from ICF signature until administration of first study drug that were not study related were reported as medical history. Concomitant medications and AEs/SAEs were recorded in the CRF through 30 days after the last dose of any study drug. Ongoing drug-related AEs were followed until resolution unless none were expected. New anti-cancer medications were captured through completion of survival follow-up.

TABLE 6
Schedule Of Study Procedures

| | | | | | | | | | | | | | Long Term |
|---|---|---|---|---|---|---|---|---|---|---|---|---|---|
| | Screening | | | | | | | | | Initial Follow-up | | | Follow-up |
| | Period | Treatment Period | | | | | | | | Period | | | Period |
| | Day | Day | Day | Day | Day | Day | Day | Day | Day | Day | Day | Day | Every |
| Activity | −28 to −1 | 0 | 1 | 2 | 3 | 4-6 | 7 | 8-13 | 14 | 15 | 28 ± 7 | 56 ± 7 | 8 weeks |
| Clinical Assessments | | | | | | | | | | | | | |
| Informed Consent | X | | | | | | | | | | | | |
| Medical/Cancer History^{a,b} | X | | | | | | | | | | | | |
| Physical Exam^{c}, | X | X | X | X | X | | X | | X | | X | | |
| including targeted | | | | | | | | | | | | | |
| neurological exam | | | | | | | | | | | | | |
| Karnofsky PS^{d} | X | X | | | | | | | X | | X | | |
| Height (only at Screen) | X | | | | | | X | | X | | X | | |
| and Weight | | | | | | | | | | | | | |
| Vital Signs^{e} | X | X | X | X | X | | X | | X | | X | | |
| Adverse Events^{f} | X | → | | | | | | | | | | X | |
| Concomitant | X | → | | | | | | | | | | X | X^{g} |
| Medications^{b,f} | | | | | | | | | | | | | |
| Survival Status^{g} | X | → | | | | | | | | | | | |
| Clinical Laboratory^{u} | | | | | | | | | | | | | |
| Pregnancy Test^{h} | X | X | | | | | | | | | | | |
| Hematology Panelⁱ | X | X | | X | X | | X | | X | | X | | |
| Coagulation Panel^{j} | X | X | | | X | | X | | X | | | | |
| Serum Chemistry Panel^{k} | X | X | | X | X | | X | | X | | X | | |
| Urinalysis Panel^{l} | X | X | | | | | | | X | | | | |
| ECG^{m} | X | X | | | X | | | | X | | | | |
| Registrationⁿ | X | | | | | | | | | | | | |
| Study Drug Administration | | | | | | | | | | | | | |
| Ad-RTS-hIL-12 | | X^{o,p} | | | | | | | | | | | |
| Veledimex Dose | | X | X^{p,q} | X | X | X | X | X | X^{r} | | | | |
| Veledimex Dose | | | X | X | X | X | X | X | X | | | | |
| Compliance/Subject | | | | | | | | | | | | | |
| Diary^{r} | | | | | | | | | | | | | |
| PK/PD/Immune Assessments | | | | | | | | | | | | | |
| Viral Shedding | X | | | | X | | | | | | | | |
| Assessment | | | | | | | | | | | | | |
| Veledimex PK blood | | X^{t} | X | X | X | | X | | X | X | | | |
| sample^{s, u} | | | | | | | | | | | | | |
| Serum Cytokine profile^{u} | | X^{t} | | | X | | X | | X | | X | | |
| Immune Function blood | | X | | | | | X | | X | | X | | |
| sample^{u} | | | | | | | | | | | | | |
| MRI Scans^{v,w} | X^{v,w} | | | X^{v,w,x} | | | | | X^{v} | | X^{w} | X^{w} | X^{w} |
| Tumor Sample^{u} | | X | | | | | | | | | | | |
| CSF Sample^{y} | | X | | | | | | | | | | | |

^{a}Medical history includes demographic information, medical and surgical history. Cancer history includes current cancer diagnosis, prior treatment [regimen(s), doses, start and stop dates and any associated residual toxicity], and best response for each regimen.
^{b}Medications received in the period preceding consent (~28 days) in addition to those ongoing at screening were captured in the CRF.
^{c}A complete physical examination including a neurological exam and mental status was required at baseline. Targeted neurological exams thereafter.
^{e}Blood pressure, pulse, temperature, and respiration were recorded. Blood pressure was monitored closely, with hydration as needed to prevent hypotension after veledimex administration. Subjects were instructed to maintain adequate oral hydration on and between veledimex dosing days; sites closely monitored subjects' hydration status.
^{f}Monitoring and recording of concomitant medications and adverse events (AEs) and serious adverse events (SAEs) was conducted throughout the study. Concomitant medications given and AEs/SAEs that occur following signed informed consent form (ICF) through the initial Follow-up Period (e.g. Day 56 visit) were recorded in the CRF. AEs that are ongoing at the end of the Initial Follow-up Period and are considered drug related were followed until resolution or no resolution was expected.
^{g}Patients were followed to document start of a new anticancer therapies and survival status for 2 years following administration of Ad-RTS-hIL-12.
^{h}Females of childbearing potential had a serum pregnancy test at the Screening Visit and a urine or serum pregnancy test on Day 0, with a negative pregnancy outcome required prior to first dose of study drug
ⁱHematology Panel: complete blood count, white blood count with differential, red blood cell count, hematocrit, hemoglobin, red blood cell indices, mean corpuscular volume, and platelet count.
^{j}Coagulation Panel: activated partial thromboplastin time or partial thromboplastin time, international normalized ratio, erythrocyte sedimentation rate and C-reactive protein.
^{k}Serum Chemistry Panel: aspartate transaminase, alanine transaminase, lactate dehydrogenase, alkaline phosphatase, creatinine, total bilirubin, total
protein, albumin, amylase, blood urea nitrogen, glucose, sodium, potassium, chloride, calcium, phosphorus and bicarbonate.
^{l}Urinalysis Panel (dipstick): appearance, pH, specific gravity, glucose, protein/albumin, blood, ketones, bilirubin, nitrates, and leukocyte esterase. In addition, a microscopic exam for casts, crystals and cells were done if clinically indicated.
^{m}Standard 12-lead ECG; single measurement at each time point
ⁿCentralized registration of eligible subjects was completed prior to first dose of study drug according to a process defined by the sponsor.
^{o}Ad-RTS-hIL-12 intratumoral injection was administered by freehand injection. Subjects were instructed to maintain adequate oral hydration during the Treatment Period; sites closely monitored subjects' hydration status. Because of the potential for toxicity (e.g., fevers, chills, fatigue and dehydration), administration of prophylactic antipyretics was recommended after injection of Ad-RTS-hIL-12.
^{p}Each subject was carefully monitored for any local reactions and/or hypersensitivity reactions following the Ad-RTS-hIL-12 injection and veledimex administration. Subjects were instructed to call the clinical site if headache, hemiparesis, seizure or other local reactions develop anytime and especially between study visits.
^{q}The first postresection veledimex dose was given on the next day, designated as Day 1, preferably with food. Subsequent veledimex doses were taken once daily, in the morning and within 30 minutes of a regular meal.
^{r}Study sites determined compliance of veledimex dosing. Subjects were instructed to document veledimex dosing compliance in a subject diary, including the time subject took the once daily dose, the time subject ate breakfast, the number of capsules taken, whether subject missed any veledimex doses and the study day and reason for any missed doses. Study drug container(s) with any remaining capsules were returned to the study staff, so that staff could properly assess dose compliance.
^{s}The PK sampling schedule is provided in Table 3.
^{t}Day 0, PK, and cytokine blood samples were obtained prior to Ad-RTS-hIL-12 injection.
^{u}Additional ad hoc samples, including blood, tissue, excretions, etc., could be requested to evaluate safety. In the event of a drug-related AE, if possible, an unscheduled visit kit was obtained for cytokines and immunological markers for CSF evaluation, if applicable.
^{v}Appropriate cancer staging procedures were performed during screening. All imaging were of diagnostic quality. The brain was imaged using the same method(s) used throughout the study. Measurable target lesions were selected and measured per RANO/iRANO guidelines. A repeat scan to confirm progression was completed at 4 weeks (per RANO) and preferably again at 12 weeks (per iRANO) after first documentation of progression. Additional tumor response assessments as well as a posttreatment diagnostic brain biopsy were performed at the discretion of the investigator as part of providing standard of care treatment in accordance with current iRANO guidelines.
^{w}The Day 28 (±7 days) and Day 56 (±7 days) MRI scans were required for all subjects, including those with unconfirmed disease progression, to ensure that more slowly declining tumor burden in response to therapy was noted. For 2 years, subjects without confirmed disease progression continued to have tumor assessments performed every 8 weeks as per standard practice until disease progression had been identified (first documentation) and confirmed (12 weeks after first documentation). MRI scans were available for collection upon sponsor request.
^{x}The MRI scan designated on Day 2 was taken within 72 hours of Ad-RTS-hIL-12 administration and was considered the baseline scan for tumor response assessments.
^{y}Additional tumor, CSF (if available) and blood samples were collected, if available, as part of standard of care procedures.

### Investigational Products

The investigational product was comprised of two components: Ad-RTS-hIL-12 and veledimex. Ad-RTS-hIL-12 is administered intratumorally or at the margin of the tumor, veledimex is administered PO.

### Preparation of Product for Administration

### Preparation of Ad-RTS-hIL-12

Ad-RTS-hIL-12 was supplied in single-dose vials. Information regarding the preparation of the Ad-RTS-hIL-12 dose was provided in the Pharmacy Manual.

### Preparation of Veledimex

The Sponsor provided veledimex capsules that were dispensed by the study site pharmacy to subjects for PO administration.

### Treatment Plan

### Ad-RTS-hIL-12+Veledimex

Subjects were given a 20 mg dose of veledimex by mouth, on an empty stomach (excluding other medications) 3 (±2) hours before craniotomy (Day 0). The actual time of veledimex administration was noted and recorded.

Surgical planning was performed on a diagnostic MRI acquired prior to the surgery as per standard of care.

At the time of tumor resection, tumor, CSF (if available), and blood samples were collected.

Immediately after tumor resection, when available, an intraoperative MRI could be performed to identify contrast enhancing or T2/FLAIR hyper intense residual tumor. If intraoperative MRI was not available, the neurosurgeon would select sites for injection.

Subjects received Ad-RTS-hIL-12 2×10¹¹ vp. This was administered by freehand injection into approximately two sites within the residual tumor for a total volume of 0.1 mL selected by the neurosurgeon. When available an intra-operative MRI was performed to guide the Ad-RTS-hIL-12 injection to areas of contrast-enhancing tumor tissue.

The day of Ad-RTS-hIL-12 administration was designated as Day 0. In the event that Ad-RTS-hIL-12 injection was not performed, subject did not continue with postresection veledimex dosing.

After tumor resection and Ad-RTS-hIL-12 injection, 20 mg of veledimex was administered orally for 14 days. The first postresection veledimex dose was given on Day 1, preferably with food. Subsequent veledimex doses were taken once daily, in the morning and within approximately 30 minutes of a regular meal.

Subjects were carefully monitored for possible local reactions and/or hypersensitivity reactions, according to standard practice. Intracranial bleeding or other procedure-related events were evaluated before the first veledimex dose was given post Ad-RTS-hIL-12 administration. Any changes in neurological status was reported to the investigator immediately, either during hospitalization or once subject was discharged. Subjects were instructed to call the study physician or study nurse if they developed any symptoms after they were released from the hospital.

Administration of prophylactic antipyretics was recommended during the first week after Ad-RTS-hIL-12 injection.

### Stopping Rules

If any subject, in the treatment and initial follow up period, experienced a local reaction requiring operative intervention; a local reaction that has life-threatening consequences requiring urgent intervention or results in death; or a Grade 4 hematologic toxicity that persists for 5 days; or death (other than death related to progressive disease) that occurs within 30 days of dosing, enrollment of new subjects would be paused pending review of the event by the Safety Review Committee (SRC).

If any subject, in the treatment and initial follow up period, experienced a local reaction that required operative intervention or a local reaction that has life-threatening consequences requiring urgent intervention or results in death the treating site's Principal Investigator and the Medical Monitor would discuss the relationship to study drug and determine whether or not to convene an urgent SRC meeting to make a decision to continue active dosing in ongoing subjects.

### Dose Modifications and Dose Delays

Dose modifications and delays for veledimex are described in the Dose Modifications and Dose Delays section of the main protocol of Example 1.

### Endpoints

### Primary Endpoint

The primary endpoint was the assessment of safety and tolerability (as determined by adverse event rate) of Ad-RTS-hIL-12, administered by intratumoral injection plus veledimex.

### Secondary Endpoints

Secondary Endpoints were as Follows:

Investigator assessment of ORR and PFS

OS

Veledimex concentration ratio between brain tumor, CSF, and blood

Veledimex PK estimates starting Day 1, as previously described

Correlative measures of immune response including serum cytokine levels and immune cell populations in the tumor.

### Analyses

Analyses are described in the Analyses section of the main protocol of Example 1

### Procedures for Reporting Deviations to Original Statistical Analysis Plan

Procedures for reporting deviations to the original statistical analysis plan are described in the Procedures for Reporting Deviations to Original Statistical Analysis Plan section of the main protocol of Example 1.

### Adverse Event Reporting

Adverse event reporting would follow that of the main protocol of Example 1.

TABLE 7
List Of Abbreviations And Definitions Of Terms

| Abbreviation or | |
|---|---|
| Specialist Term | Explanation |
| Ad-RTS- | Adenovirus RheoSwitch Therapeutic System ® human |
| hIL-12 | interleukin-12 |
| AE | adverse event |
| ALP | alkaline phosphatase |
| ALT | alanine transaminase |
| aPTT | activated partial thromboplastin time |
| ASA | acetylsalicylic acid |
| AST | aspartate transaminase |
| BSA | body surface area |
| BUN | blood urea nitrogen |
| CBC | complete blood count |
| CD | cluster of differentiation |
| CRF | case report form |
| CRP | C-reactive protein |
| CSF | cerebrospinal fluid |
| CTCAE | Common Terminology Criteria for Adverse Events |
| CTLA-4 | cytotoxic T lymphocyte-associated antigen 4 |
| CYP450 | cytochrome p450 |
| DIPG | diffuse intrinsic pontine glioma |
| DLT | dose-limiting toxicity |
| DSMB | Data and Safety Monitoring Board |
| ECG | Electrocardiogram |
| ECOG | Eastern Cooperative Oncology Group |
| EIAED | antiepileptic drugs |
| ESP | Evaluable Safety Population |
| EXP | expansion |
| FDA | Food and Drug Administration |
| GCP | Good Clinical Practice |
| hIL-12 | human interleukin-12 |
| HIV | human immunodeficiency virus |
| ICF | informed consent form |
| ICH | International Conference on Harmonization |
| IFN-γ | interferon-gamma |
| INR | international normalized ratio |
| IP-10 | IFN-γ-induced protein 10 |
| iRANO | Immunotherapy Response Assessment for Neuro- |
| | Oncology |
| IV | intravenous(ly) |
| MRI | magnetic resonance imaging |
| MTD | maximum tolerated dose |
| NCI | National Cancer Institute |
| ORR | objective response rate |
| OS | overall survival |
| OSP | Overall Safety Population |
| PFS | progression -free survival |
| PI | Principal Investigator |
| PK | pharmacokinetic(s) |
| PKP | Pharmacokinetics Population |
| PO | oral(ly) |
| QD | once daily |
| rAd | recombinant adenovirus |
| RANO | Response Assessment for Neuro-Oncology |
| rhIL-12 | recombinant human IL-12 |
| RTS | RheoSwitch Therapeutic System ® |
| RXR | retinoid X receptor |
| SAE | serious adverse event |
| SRC | Safety Review Committee |
| TEAE | treatment-emergent adverse event |
| ULN | upper limit of normal |
| Vp | viral particles |
| WBC | white blood cell |

### Example 3: Intratumoral Administration of AD-RTS-HIL-12 and Veledimex in Subjects with Recurrent Glioblastoma

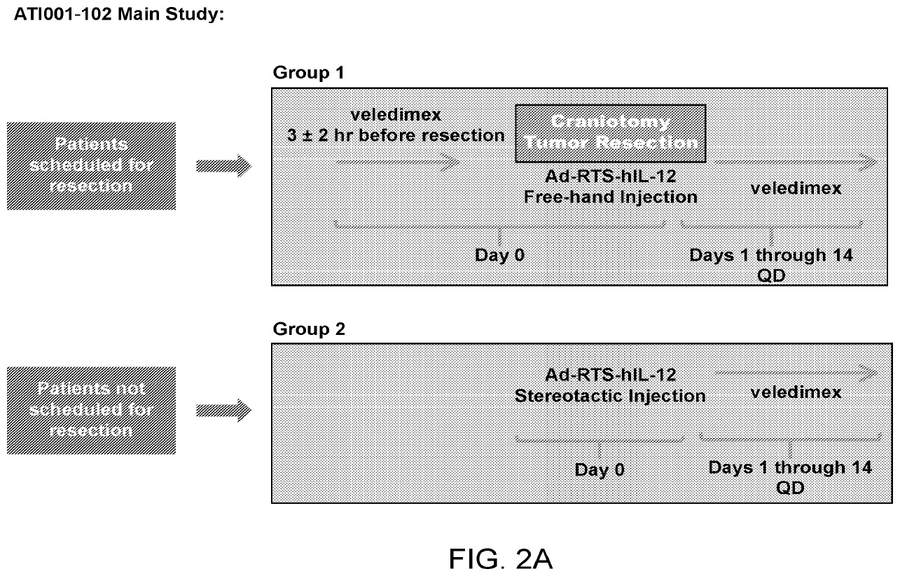

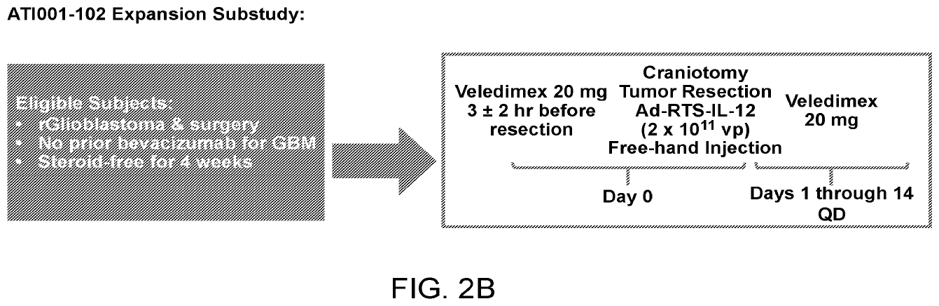

Abstract Ad-RTS-hIL-12 (Ad) is a novel gene therapy, conditionally expressing IL-12 via the RheoSwitch Therapeutic System® (RTS®) gene switch under control of an oral activator ligand, veledimex (V). Two studies were performed—Main Study (FIG. 2A) and Expansion Substudy (FIG. 2B). We previously reported results from 51 subjects describing biological activity of controlled IL-12, safety and survival data. Previously, subjects who received Ad+V (20 mg) managed with low-dose dexamethasone in the Main study achieved a mOS of 17.8 months, which is approximately twice the anticipated survival compared to historical controls. The mechanism of action of Ad+V is based on controlled secretion of recombinant IL-12 (measured in peripheral blood as a surrogate for intra-tumor-production), downstream upregulation of endogenous IFN-γ (measured in peripheral blood) and an increase in the “cytoindex” (ratio of circulating CD8⁺ T cells to FoxP3⁺ regulatory T cells), an emerging biomarker of overall survival. Herein we provide an update of subject characteristics, survival and biomarker analysis from the Phase 1 and Expansion Substudy. Assessing safety and tolerability of local, inducible IL-12 by single intratumoral injection of Ad (2×10¹¹ viral particles)+V (20 mg PO QD×15 doses during Days 0-14) in subjects, including a subset receiving low-dose corticosteroids (≤20 mg cumulative dexamethasone during Days 0-14). Drug-related toxicities were predictable, dose-related and promptly reversible upon discontinuation of V with no drug-related deaths. Biomarker studies related to production of IL-12 and IFN-γ, as well as cytoindex remain encouraging. As of Jun. 4, 2019, mOS in the Expansion Substudy had not yet been reached. Most subjects (65%) received low-dose dexamethasone (cumulative ≤20 mg Days 0-14); initial impact of this and other subject characteristics on survival are described below.

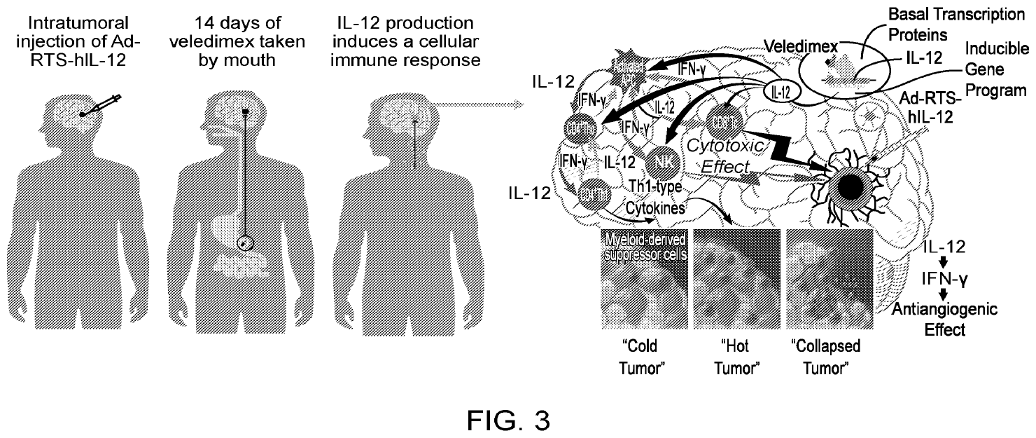

Background on Controllable IL-12. Ad-RTS-hIL-12 (Ad) is a recombinant, adenoviral-delivered, gene therapy for expression of interleukin-12 (IL-12) under the control of an orally administered activator ligand, veledimex (V), acting in concert with a ligand-inducible gene switch (also referred to as RheoSwitch Therapeutic System® or “RTS®”). Administration of Ad-RTS-hIL-12 provides for elicitation of an anti-cancer effector T cell response while concurrently providing ability to control and/or reduce adverse effects which may be caused by IL-12 over-expression and/or an undesirable degree of IL-12 systemic toxicity. Gene Switch Components: RheoSwitch Therapeutic System® (RTS®) technology includes VP16-RXR (co-activation partner, CAP) and Gal4-EcR (ligand-inducible transcription factor, LTF. Without ligand, LTF binds to the inducible promoter and does not form a stable complex with CAP. Inducible Promoter: Customizable (RTS®) promoter to which basal transcription proteins are recruited and the target gene (IL-12) is transcribed. Activator Ligand (veledimex): After oral administration, this ligand, an ecdysone analog, stabilizes a conformational change in the LTF leads to a stable, high-affinity interaction with CAP. Ad-RTS-hIL-12 intratumoral injection regulated by veledimex drives downstream production of endogenous IFN-γ and elicits a brisk cytotoxic immune response (FIG. 3). Systemic corticosteroids are routinely used to treat edema in patients with intracranial tumors. However, it is not well studied if or when corticosteroids can be administered without abrogating the benefits of immunotherapy.

### Safety Results

TABLE 8
Safety Results of Main Study and Expansion Substudy

| | | |
|---|---|---|
| | Main Study | Expansion Substudy |
| Adverse Events | 20 mg (N = 15) | 20 mg (N = 36) |

Related ≥Grade 3 AEs in ≥5% of Subjects

| | | | |
|---|---|---|---|
| Lymphopenia | 2 | (13%) | 5 (14%) |
| Thrombocytopenia | 2 | (13%) | 0 |
| Leukopenia | 1 | (7%) | 2 (6%) |
| Neutropenia | 1 | (7%) | 2 (6%) |
| AST/ALT increased | 1 | (7%) | 1 (3%) |
| Headache | 2 | (13%) | 1 (3%) |
| Meningitis Aseptic | 1 | (7%) | 0 |
| Hyponatremia | 2 | (13%) | 0 |
| Amylase increased | 1 | (7%) | 1 (3%) |

| | | |
|---|---|---|
| Mental Status Change | 0 | 1 (3%) |

Related Serious Adverse Events (SAEs)

| Pyrexia | 1 | (7%) | 1 (3%) |
|---|---|---|---|
| Cytokine Release Syndrome | 2 | (13%) | 0 |
| Thrombocytopenia | 2 | (13%) | 0 |
| Neutropenia | 1 | (7%) | 1 (3%) |
| Leukopenia | 1 | (7%) | 0 |
| AST/ALT increased | 1 | (7%) | 0 |
| Meningitis Aseptic | 1 | (7%) | 0 |
| Mental Status Change | 0 | | 1 (3%) |
| Headache | 1 | (7%) | 0 |
| Nausea | 2 | (14%) | 0 |

| | | |
|---|---|---|
| Seizure | 0 | 1 (3%) |

Cytokine Release Syndrome²

| | | | |
|---|---|---|---|
| Grade 2 | 12 | (80%) | 24 (67%) |
| Grade 3 | 1 | (8%) | 2 (6%) |

¹Data collection and follow-up are ongoing.
²Ziopharm Cytokine Release Syndrome Working Definition

### Study Designs—Substudy Rationale and Inclusion Criteria

Two studies were performed—Main Study (FIG. 2A) and Expansion Substudy (FIG. 2B). 31 subjects undergoing craniotomy were enrolled at four dose levels of veledimex (10, 20, 30 and 40 mg) in the Main Study, with 15 treated with 20 mg. Median overall survival (mOS) at this dose level was 12.7 mos. (mean follow up of 13.1 mos.). Ad hoc analysis of steroid use during active treatment in the Main Study showed a negative impact on mOS in the 20 mg cohort for subjects that received a cumulative dose of >20 mg of concurrent steroids during active V dosing (Days 0-14). Expansion substudy inclusion criteria were kept similar to the Main Study with the addition of the following modifications: Expansion substudy subjects were (1) required to have a diagnosis of recurrent glioblastoma; (2) required to be steroid-free for 4 weeks prior to study entry; and (3) required to be bevacizumab-naïve for disease treatment.

### Subject Characteristics

As expected, based on study design, there was a higher percentage of subjects in the Expansion substudy as compared with Main Study (75% vs 40%) who received low-dose steroids (≤20 mg dexamethasone total, Days 0-14) concurrent with veledimex dosing. A higher percentage of subjects in the substudy as compared with the Main Study (39% vs 7%) had multifocal disease vs. unifocal disease at study entry. In some embodiments, multifocal disease is shown through the identification of multiple enhancing lesions in MRI. In some embodiments, unifocal disease is shown through the identification of one enhancing lesion in MRI. Additionally, a high percentage of subjects in the substudy had more than one lesion (enhancing and non-enhancing lesions on MRI) compared to the Main Study (69% vs 14%). Multifocal glioblastoma is associated with a worse prognosis compared to unifocal disease (Cancer Manag Res. 2018; 10:4229-4235 and Int. J. Mol. Sci. 2017, 18, 2469). A higher percentage of subjects in the substudy as compared with the Main study (75% vs. 20%) had a 1^{st} recurrence vs. recurrences.

TABLE 9
Subject Characteristics

| | Main Study | | Expansion Substudy | |
|---|---|---|---|---|
| Subject Characteristics¹ | 20 mg (N = 15) | | 20 mg (N = 36) | |
| Age, years (Mean, Range) | 46.5 | (26, 68) | 51.5 | (21, 72) |
| Gender, N (%) | | | | |
| Male | 10 | (67%) | 22 | (61%) |
| Female | 5 | (33%) | 14 | (39%) |
| Disease status at entry² | | | | |
| Unifocal | 14 | (93%) | 22 | (61%) |
| Multifocal | 1 | (7%) | 14 | (39%) |
| Number of Lesions at Entry³ | | | | |
| 1 | 13 | (86%) | 11 | (31%) |
| 2 | 1 | (7%) | 18 | (50%) |
| 3+ | 1 | (7%) | 7 | (19%) |
| Number of recurrences | 3 | (20%) | 27 | (75%) |
| 1^{st} recurrence | 12 | (80%) | 7 | (19%) |
| ≥2 recurrences | 0 | | 2 | (6%) |
| Prior Lines of Treatment (mean) | 2.3 | (1, 4) | 1.4 | (1, 3) |
| IDH Status, N (%) | 8 | (53%) | 28 | (78%) |
| Wild-Type | 6 | (40%) | 1 | (3%) |
| Mutated | 1 | (7%) | 7 | (19%) |
| Methylation Status, N (%) | 7 | (47%) | 10 | (28%) |
| Methylated | 6 | (40%) | 23 | (64%) |
| Unmethylated | 2 | (13%) | 3 | (8%) |
| KPS at Screening, N (%) | | | | |
| ≥70-90 | 6 | (40%) | 13 | (36%) |
| ≥90 | 9 | (60%) | 23 | (64%) |
| Cumulative Steroid Use, Days 0-14 (mg) | 60 | (0, 140) | 23.2 | (0, 166) |
| (Mean, Range) | | | | |
| Concurrent steroid use | | | | |
| ≤20 mg dexamethasone total, Days 0-14 | 6 | (40%) | 27 | (75%) |
| >20 mg dexamethasone total, Days 0-14 | 9 | (60%) | 9 | (25%) |
| Veledimex Dosing Compliance (%) | 84% | | 93% | |
| V qd (for 15 days) | | | | |

¹Data collection and follow-up are ongoing: Data as of 14 Nov. 2019.
²Based on number of reported enhancing lesions.
³Based on number of enhancing and non-enhancing lesions.

### Serum Cytokine Production Sustained During Dosing and Increase in Cytoindex Indicate Immune Activation (Main and Substudy)

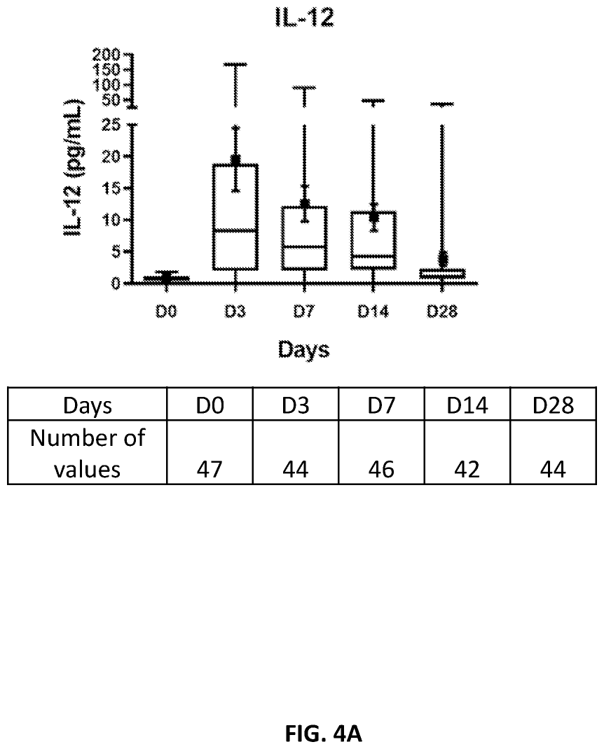

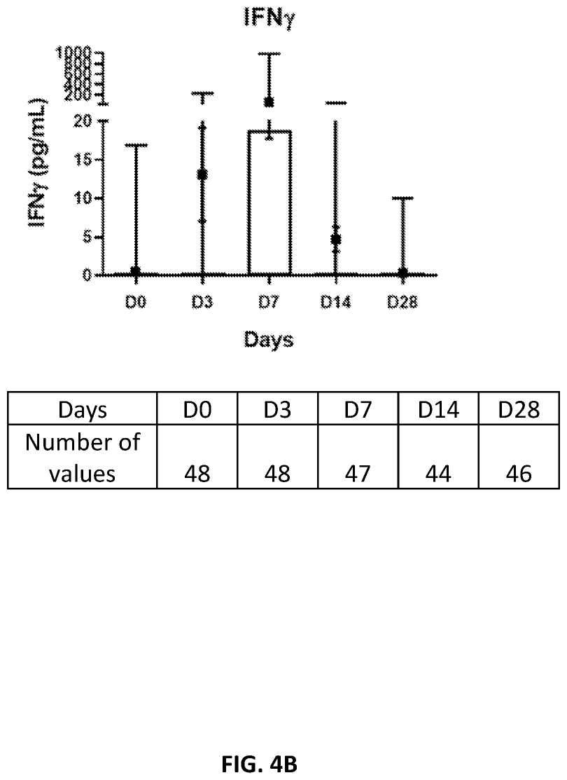

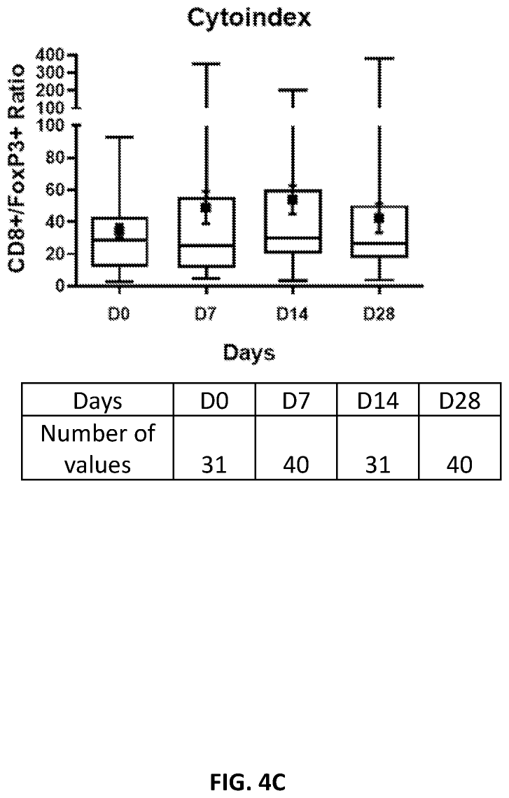

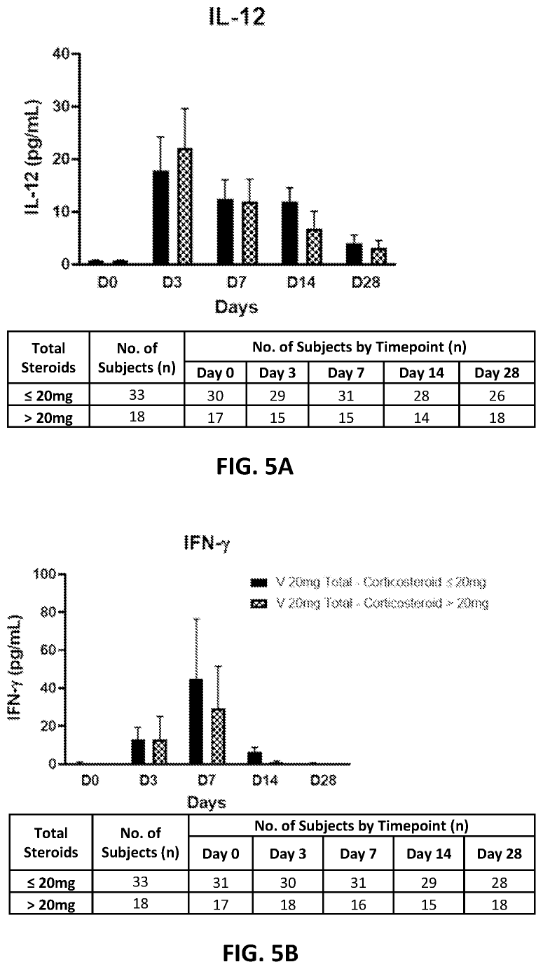

Day 3 peak in serum IL-12 production (FIG. 4A) was followed by a peak in downstream IFN-γ at Day 7 (FIG. 4B) in virtually all subjects. IL-12-mediated activation of peripheral blood immune cells as assessed by serial measurements of the ratio of CD8⁺/FoxP3⁺ (i.e., cytotoxic/suppressor to regulatory) T cells is described as the “cytoindex” (FIG. 4C). The serum IL-12 and IFN-γ data was further segregated to compare patients administered a total of ≤20 mg dexamethasone vs. >20 mg dexamethasone during veledimex dosing (FIGS. 5A-5B). Patients administered a total of ≤20 mg dexamethasone during veledimex dosing have a particularly large increase in serum IFN-γ on Day 7.

### Evidence of Immune Mediated Anti-Tumor Response by Serial MRI

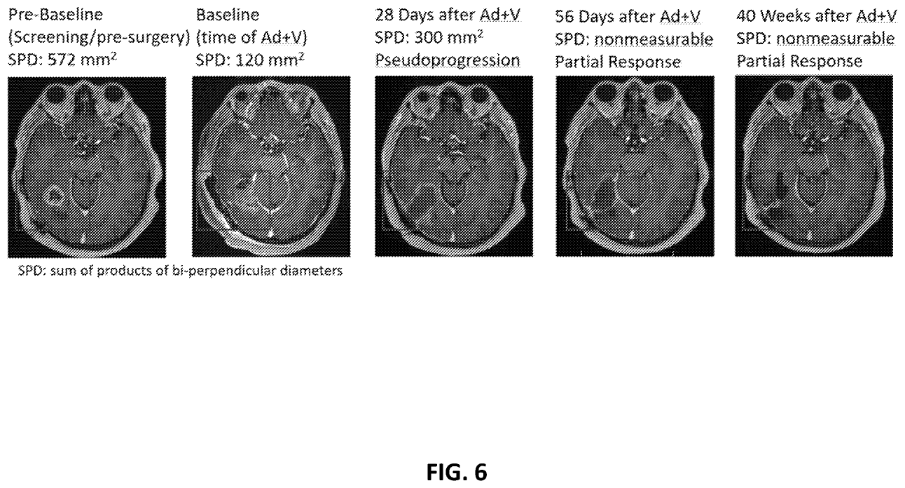

In the Main Study, 3/3 subjects underwent biopsy at suspected progression which confirmed pseudoprogression based on extensive immune (T-cell) infiltrates per Sci Transl Med. 2019 Aug. 14; 11(505). MRI scans from an Expansion subject were interpreted as pseudoprogression at Day 28 with the lesion improving and becoming non-measurable by Day 56 continuing through Week 40 (partial response per iRANO criteria), with monitoring ongoing (FIG. 6). The subject is a 51 year old female with 1 line of prior therapy; and symptoms of status epilepticus at entry. She has Unifocal disease, with 1 enhancing lesion and no non-enhancing lesions. She received 13 doses of 20 mg of veledimex with 0 mg of dexamethasone during veledimex dosing. On study, seizures well controlled with no worsening of neurological symptoms. At the time of pseudoprogression there was no worsening of clinical status.

### Survival in 20 mg Veledimex (Main Study and Expansion Substudy)

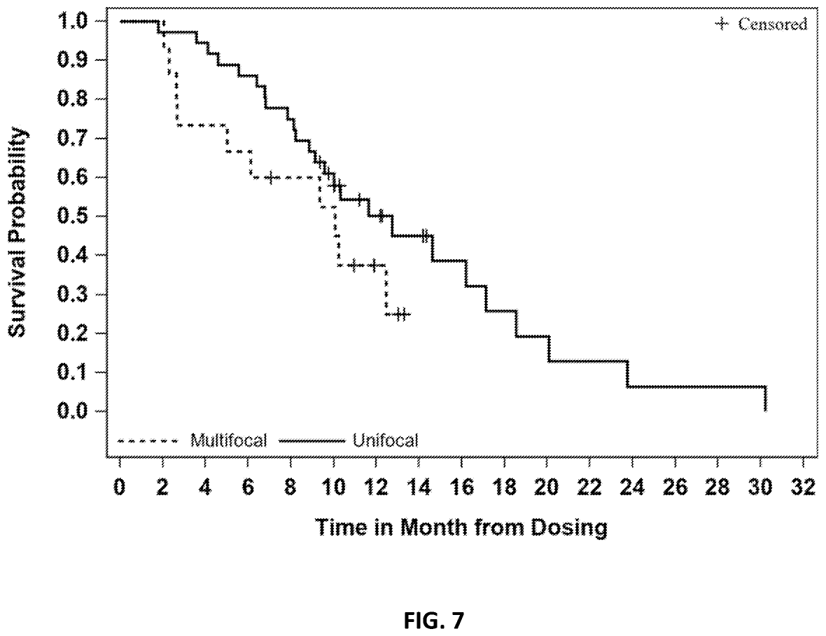

FIG. 7 and Table 10 show the survival of unifocal vs multifocal disease subjects from the main study and the expansion study (independent of steroid treatment).

TABLE 10
Survival by Unifocal vs Multifocal
Disease (independent of steroids)

| | | | Median | |
|---|---|---|---|---|
| | | No. of | Survival | Mean |
| | No. of | Subjects | (95% CI) | Follow-up |
| Cohort | Subjects | Alive | (mos.) | (mos.) |
| Unifocal | 36 | 11 | 12.7 (8.9, 17.1) | 11.1 |
| Multifocal | 15 | 5 | 10.1 (2.6, —) | 7.9 |

FIG. 8 and Table 11 show the survival of unifocal subjects by cumulative concurrent steroids. Together, this shows that 20 mg V subjects (Main+Expansion, n=20) with unifocal disease at entry, receiving low-dose concurrent steroids continued to show a trend towards longer survival.

TABLE 11
Survival of Unifocal Subjects by Cumulative Concurrent Steroids

| | | | | Median | | |
|---|---|---|---|---|---|---|
| | Cumulative | | No. of | Survival | | Mean |
| | Steroids | No. of | Subjects | (95% CI) | | Follow- |
| Cohort | (Days 0-14) | Subjects | Alive | (mos.) | | up(mos.) |
| Unifocal | ≤20 mg | 20 | 7 | 16.2 | (8.9, 18.5) | 12.3 |
| | >20 mg | 16 | 4 | 9.8 | (4.6, 30.2) | 9.7 |
| Multifocal | ≤20 mg | 13 | 5 | 10.1 | (2.6, —) | 7.5 |
| | >20 mg | 2 | 0 | 10.9 | (9.4, 12.5) | 10.9 |

### Discussion and Conclusions

Interim results of Main and Expanded 20 mg V cohorts of subjects (N=51) show efficacy for subjects with rGBM, especially for unifocal disease with low-dose steroids. Drug-related toxicities were comparable: predictable and promptly reversible upon discontinuation of veledimex and with no drug-related deaths. Immune activation was demonstrated by: 1) Peak Serum IL-12 at Day 3 with downstream endogenous IFN-γ production peak at Day 7; 2) Commensurate increase in cytoindex; 3) Serial MRI demonstrated pseudoprogression (via biopsy in the Main Study) followed by reduction in lesion to nonmeasureable (in Expansion Substudy). Expansion Substudy subjects were comparable to the Main study with the exception of number of lesions (unifocal versus multifocal) and recurrences at study entry and cumulative concurrent steroids (Days 0-14, during veledimex dosing). The Main study showed mOS in subjects (n=6, unifocal) receiving low-dose concurrent steroids (17.8 months). The mOS had not been reached for this same group in the Expansion Substudy. 20 mg V subjects (Main+Expansion, n=20) with unifocal disease at entry, receiving low-dose concurrent steroids continued to show a trend towards longer survival (16.2 mos.).

### Example 4: Intratumoral Administration of AD-RTS-HIL-12 and Veledimex in Subjects with Recurrent or Progressive Glioblastoma or Grade III Malignant Glioma—Main Study Full Cohort Data

The data from the Main Study described above was expanded below to include the full Group 1 and Group 2 cohorts. Group 1 subjects received one veledimex dose before a standard-of-care resection procedure (i.e., craniotomy) and Ad (2×10¹¹ viral particles) was administered by freehand intratumoral injection, then subjects continued with oral veledimex orally daily for 14 days. The subjects in Group 1 were enrolled at four dose levels of veledimex (10, 20, 30 and 40 mg) in the Main Study. The subjects in Group 2 were not scheduled for tumor resection and received Ad by stereotactic injection and then continued on oral veledimex (20 mg) daily for 14 days.

### Subjects Undergoing Tumor Resection (Group 1):

Subjects were given a specific dose of veledimex by mouth, on an empty stomach (excluding other medications) 3 (±2) hours before craniotomy. The actual time of veledimex administration was to be noted and recorded.

Surgical planning was performed on a diagnostic MRI acquired prior to the surgery as per standard of care.

At the time of tumor resection, tumor, CSF (if available) and blood samples were collected.

Immediately after tumor resection, when available, an intraoperative MRI was performed to identify contrast enhancing or T2/FLAIR hyper intense residual tumor. If intraoperative MRI was not available, the neurosurgeon selected sites for injection.

Subjects received Ad-RTS-hIL-12 2×10¹¹ vp. This was administered by freehand injection into approximately two sites within the residual tumor for a total volume of 0.1 mL selected by the neurosurgeon. When available an intra operative MRI was performed to guide the Ad-RTS-hIL-12 injection to areas of contrast enhancing tumor tissue.

The day of Ad-RTS-hIL-12 administration is designated as Day 0.

After tumor resection and Ad-RTS-hIL-12 injection, veledimex was administered orally daily for 14 days. The first postresection veledimex dose was given on Day 1, with food. Subsequent veledimex doses were taken once daily, in the morning and within approximately 30 minutes of a regular meal.

### Subjects NOT Undergoing Tumor Resection (Group 2):

Surgical planning was performed on a diagnostic MRI acquired prior to the stereotactic procedure.

On the day of Ad-RTS-hIL-12 administration (Day 0), subjects were anesthetized and prepared for standard stereotactic surgery.

Subjects received Ad-RTS-hIL-12 2×10¹¹ vp. It was administered by stereotactic injection into approximately two intratumoral sites to deliver up to 0.1 mL volume. In case of multifocal tumors, one leading lesion was selected for injection.

After the Ad-RTS-hIL-12 injection, veledimex was administered orally daily for 14 days. The first veledimex dose was given on Day 1, preferably with food. Subsequent veledimex doses were taken once daily, in the morning and within approximately 30 minutes of a regular meal.

### Safety Results

TABLE 12
Safety Results of Main Study

| | Group 1 | | | | Group 2 |
|---|---|---|---|---|---|
| | V 10 mg | V 20 mg | V 30 mg | V 40 mg | V 20 mg |
| Adverse Events | (N = 6) | (N = 15) | (N = 4) | (N = 6) | (N = 7) |

Related ≥Grade 3 AEs in ≥5% of Subjects

| Lymphopenia | 1 (16.7) | 2 | (13.3) | 1 (25.0) | 2 (33.3) | 1 (14.3) |
|---|---|---|---|---|---|---|
| Thrombocytopenia | 0 | 2 | (13.3) | 0 | 0 | 0 |
| Leukopenia | 1 (16.7) | 1 | (6.7) | 0 | 0 | 1 (14.3) |
| Neutropenia | 1 (16.7) | 1 | (6.7) | 0 | 0 | 1 (14.3) |
| AST/ALT increased | 2 (33.3) | 0 | | 0 | 2 (33.3) | 0 |
| LFT increased | 0 | 1 | (6.7) | 0 | 1 (16.7) | 0 |
| Headache | 0 | 2 | (13.3) | 0 | 0 | 0 |
| Meningitis Aseptic | 0 | 1 | (6.7) | 0 | 0 | 0 |
| Hyponatremia | 0 | 2 | (13.3) | 0 | 1 (16.7) | 0 |
| Amylase increased | 0 | 1 | (6.7) | 0 | 0 | 0 |

| | | | | | |
|---|---|---|---|---|---|
| Seizure | 1 (16.7) | 0 | 0 | 0 | 1 (14.3) |
| CRS | 0 | 0 | 0 | 2 (3.3) | 0 |
| Confusional state | 0 | 0 | 0 | 1 (16.7) | 1 (14.3) |
| Delirium | 0 | 0 | 0 | 0 | 1 (14.3) |
| Brain Edema | 0 | 0 | 1 (25.5) | 0 | 0 |
| Cerebral hemorrhage | 0 | 0 | 1 (25.5) | 0 | 0 |

Related Serious Adverse Events (SAEs)

| | | | | | | |
|---|---|---|---|---|---|---|
| CRS | 0 | 2 | (13.3) | 1 (25.5) | 2 (33.3) | 0 |
| Thrombocytopenia | 0 | 2 | (13.3) | 0 | 1 (16.7) | 0 |
| Neutropenia | 0 | 1 | (6.7) | 0 | 0 | 1 (14.3) |
| Leukopenia | 0 | 1 | (6.7) | 0 | 0 | 0 |
| LFT increased | 0 | 1 | (6.7) | 0 | 0 | 0 |
| Meningitis Aseptic | 0 | 1 | (6.7) | 0 | 0 | 0 |
| Pyrexia | 0 | 1 | (6.7) | 0 | 0 | 0 |
| Nausea | 0 | 2 | (13.3) | 0 | 0 | 0 |
| Headache | 0 | 1 | (6.7) | 0 | 0 | 0 |

| | | | | | |
|---|---|---|---|---|---|
| Cerebral hemorrhage | 0 | 0 | 1 (25.0) | 0 | 0 |
| Seizure | 1 (16.7) | 0 | 0 | 0 | 1 (14.3) |
| Delirium | 0 | 0 | 0 | 0 | 1 (14.3) |
| Hyponatremia | 0 | 0 | 0 | 1 (16.7) | 0 |

CRS²

| | | | | | | |
|---|---|---|---|---|---|---|
| Grade 2 | 3 (50.0) | 8 | (53.3) | 2 (50.0) | 2 (33.3) | 2 (28.6) |
| Grade 3 | 0 | 3 | (20.0) | 1 (25.0) | 3 (50.0) | 1 (14.3) |

¹Data collection and ongoing.
²Ziopharm Cytokine Release Syndrome Working Definition.
SAEs reported in ≥5% subjects: CRS (13.2%); seizure (10.5%); thrombocytopenia, hyponatremia and headache (7.9% each); and neutropenia, nausea, meningitis aseptic and encephalopathy (5.3% each). Related SAEs reported in ≥5% of subjects: CRS (13.2%), thrombocytopenia (7.9%) and neutropenia (5.3%).

TABLE 13
Subject Characteristics

| | Group 1 | | | | | | | | Group 2 | |
|---|---|---|---|---|---|---|---|---|---|---|
| Subject | 10 mg V | | 20 mg V | | 30 mg V | | 40 mg V | | 20 mg V | |
| Characteristics¹ | (N = 6) | | (N = 15) | | (N = 4) | | (N = 6) | | (N = 7) | |
| Age, years (Mean, Range) | 49 | (29, 61) | 46 | (26, 68) | 60 | (43, 74) | 48 | (36, 58) | 51 | (28, 73) |
| Gender, N (%) | | | | | | | | | | |
| Male | 3 | (50%) | 10 | (67%) | 2 | (50%) | 4 | (67%) | 5 | (71%) |
| Female | 3 | (50%) | 5 | (33%) | 2 | (50%) | 2 | (37%) | 2 | (29%) |
| Disease status at entry² | | | | | | | | | | |
| Unifocal | 5 | (83%) | 14 | (93%) | 3 | (75%) | 5 | (83%) | 6 | (86%) |
| Multifocal | 1 | (17%) | 1 | (7%) | 1 | (25%) | 1 | (17%) | 1 | (14%) |
| Number of Lesions at | | | | | | | | | | |
| Entry³ | | | | | | | | | | |
| 1 | 2 | (33%) | 13 | (87%) | 3 | (75%) | 5 | (83%) | 4 | (57%) |
| 2 | 4 | (67%) | 1 | (7%) | 1 | (25%) | 1 | (17%) | 3 | (43%) |
| 3+ | 0 | | 1 | (7%) | 0 | | 0 | | 0 | |
| Number of recurrences | | | | | | | | | | |
| 1^{st} recurrence | 5 | (83%) | 14 | (93%) | 3 | (50%) | 5 | (83%) | 6 | (86%) |
| ≥2 recurrences | 1 | (17%) | 1 | (7%) | 1 | (17%) | 1 | (17%) | 1 | (17%) |
| Prior Lines of Treatment | 2.0 | | 2.3 | | 2.8 | | 2.3 | | 2.4 | |
| (mean) | | | | | | | | | | |
| IDH Status, N (%) | | | | | | | | | | |
| Wild-Type | 5 | (83%) | 8 | (53%) | 2 | (50%) | 3 | (50%) | 2 | (29%) |
| Mutated | 1 | (17%) | 6 | (40%) | 0 | | 2 | (33%) | 0 | |
| TBD | 0 | | 1 | (7%) | 2 | (50%) | 1 | (17%) | 5 | (71%) |
| Methylation Status, N (%) | | | | | | | | | | |
| Methylated | 1 | (17%) | 5 | (33%) | 2 | (33%) | 5 | (83%) | 3 | (43%) |
| Unmethylated | 2 | (33%) | 2 | (13%) | 0 | | 1 | (17%) | 3 | (43%) |
| TBD | 2 | (33%) | 8 | (53%) | 2 | (33%) | 0 | | 1 | (14%) |
| KPS at Screening, N (%) | | | | | | | | | | |
| ≥70-90 | 3 | | 9 | | 3 | | 2 | | 3 | |
| ≥90 | 3 | | 6 | | 1 | | 4 | | 4 | |
| Cumulative Steroid Use, | 61 | (0, 102) | 60 | (0, 140) | 87 | (0-136) | 45 | (10-102) | 51 | (0, 163) |
| Days 0-14, Mean (Range) | | | | | | | | | | |
| (mg) | | | | | | | | | | |
| Concurrent steroid use | | | | | | | | | | |
| ≤20 mg dexamethasone | 2 | (33%) | 6 | (40%) | 1 | (25%) | 1 | (17%) | 4 | (57%) |
| total, Days 0-14 | | | | | | | | | | |
| >20 mg dexamethasone | 4 | (67%) | 9 | (60%) | 3 | (75%) | 5 | (83%) | 3 | (43%) |
| total, Days 0-14 | | | | | | | | | | |
| Veledimex Dosing | | | | | | | | | | |
| Compliance (%) | 79% | | 84% | | 63% | | 58% | | 90% | |
| V qd (for 15 days) | | | | | | | | | | |

¹Data collection and follow-up are ongoing: Data as of 24 Apr. 2020.
²Based on number of reported enhancing lesions.
³Based on number of enhancing and non-enhancing lesions.

### Inclusion Criteria

Male or female subject ≥18 and ≤75 years of age.

Provision of written informed consent for tumor resection, stereotactic surgery, tumor biopsy, samples collection and treatment with investigational products prior to undergoing any study specific procedures.

Histologically confirmed supratentorial glioblastoma or other WHO Grade III or IV malignant glioma from archival tissue.

Evidence of tumor recurrence/progression by MRI according to response assessment in neuro oncology (RANO) criteria after standard initial therapy.

Previous standard of care antitumor treatment including surgery and/or biopsy and chemoradiation. At the time of registration, subjects must have recovered from the toxic effects of previous treatments as determined by the treating physician. The washout periods from prior therapies are intended as follows: (windows other than what is listed below should be allowed only after consultation with the Medical Monitor).

Nitrosoureas: 6 weeks; other cytotoxic agents: 4 weeks; antiangiogenic agents including bevacizumab: 4 weeks; targeted agents including small molecule tyrosine kinase inhibitors: 2 weeks; experimental immunotherapies (e.g., PD 1, CTLA 4): 3 months; and vaccine based therapy: 3 months.

Able to undergo standard MRI scans with contrast agent before enrollment and after treatment

Karnofsky performance status ≥70

Adequate bone marrow, liver and kidney function, as assessed by the following laboratory requirements:

Hemoglobin ≥9 g/L; lymphocytes >500/mm³; absolute neutrophil count ≥1,500/mm³; platelets ≥100,000/mm³; serum creatinine ≤1.5× upper limit of normal (ULN); aspartate transaminase (AST) and alanine transaminase (ALT)≤2.5×ULN; for subjects with documented liver metastases, ALT and AST≤5×ULN; total bilirubin <1.5×ULN; and international normalized ratio (INR) and aPTT normal institutional limits.

Male and female subjects must agree to use a highly reliable method of birth control (expected failure rate less than 5% per year) from the Screening Visit through 28 days after the last dose of study drug. Women of childbearing potential (perimenopausal women must be amenorrheic for at least 12 months to be considered of non-childbearing potential) must have a negative pregnancy test at screening.

### Exclusion Criteria

Radiotherapy treatment within 4 weeks or less prior to veledimex dosing.

Subjects with clinically significant increased intracranial pressure (e.g., impending herniation or requirement for immediate palliative treatment) or uncontrolled seizures.

Known immunosuppressive disease, autoimmune conditions, and/or chronic viral infections [e.g., human immunodeficiency virus (HIV), hepatitis].

Use of systemic antibacterial, antifungal or antiviral medication for the treatment of acute clinically significant infection within 2 weeks of first veledimex dose. Concomitant therapy for chronic infections is not allowed. Subject must be afebrile prior to Ad-RTS-hIL-12 injection; only prophylactic antibiotic use is allowed perioperatively.

Use of enzyme inducing antiepileptic drugs (EIAED) within 7 days prior to the first dose of study drug. Note: Levetiracetam (Keppra®) is not an EIAED and is allowed.

Other concurrent clinically active malignant disease, requiring treatment, with the exception of non melanoma cancers of the skin or carcinoma in situ of the cervix or nonmetastatic prostate cancer.

Nursing or pregnant females.

Prior exposure to veledimex.

Use of medications that induce, inhibit or are substrates of cytochrome p450 (CYP450) 3A4 within 7 days prior to veledimex dosing without consultation with the Medical Monitor.

Presence of any contraindication for a neurosurgical procedure.

Unstable or clinically significant concurrent medical condition that would, in the opinion of the investigator or medical monitor, jeopardize the safety of a subject and/or their compliance with the protocol. Examples include, but are not limited to: unstable angina, congestive heart failure, myocardial infarction within 2 months of screening, ongoing maintenance therapy for life threatening ventricular arrhythmia or uncontrolled asthma.

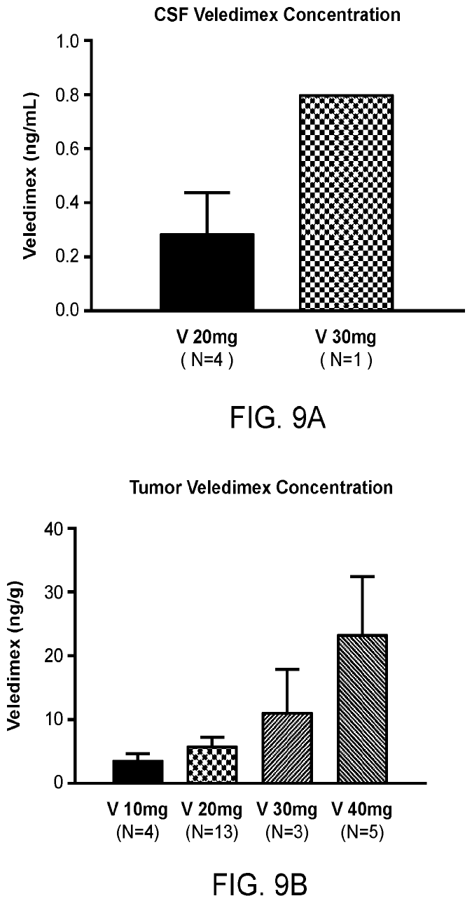

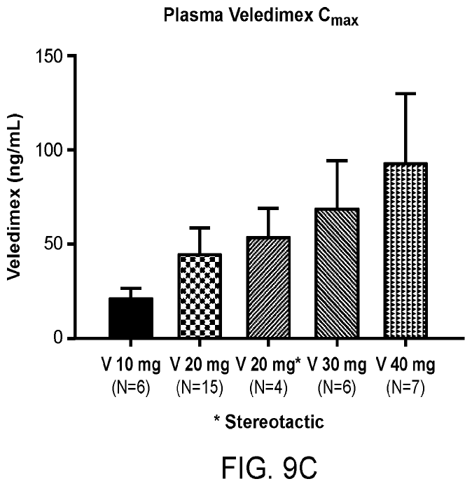

Veledimex Concentrations (Main Study). On Day 0, veledimex was administered 3±2 hours prior to surgical resection. LC-MS/MS assays for veledimex and its two major metabolites RG-116041 and RG-116043 were performed. CSF samples were collected during surgery, available from the V 20 mg and V 30 mg dose cohorts (FIG. 9A). Frozen tumor tissues were collected during surgery from 25 subjects and were analyzed for concentration of veledimex (FIG. 9B) and its two major metabolites. Plasma samples from 38 subjects were collected between 3 to 5 hours after veledimex dosing and were analyzed for veledimex Cₘₐₓ (FIG. 9C).

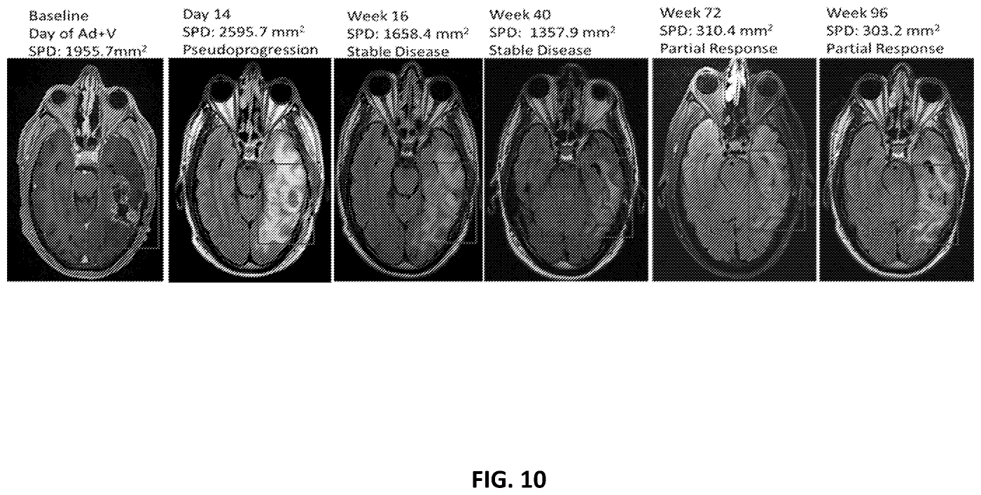

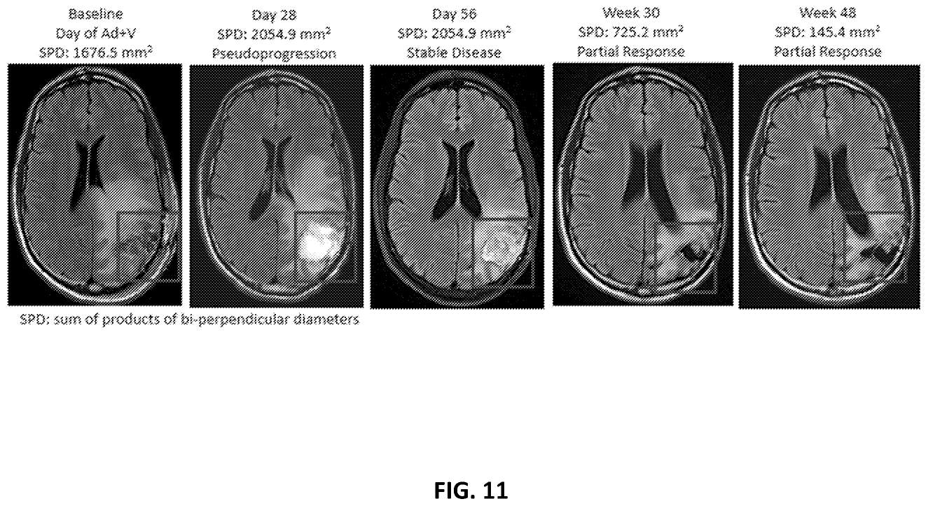

Evidence of Immune Mediated Anti-tumor Response by Serial MRI. MRI scans from two Main Study unifocal subjects were generated to demonstrate evidence of an anti-tumor response. The first subject was a 51 year old male with unifocal disease with one enhancing lesion and one prior line of therapy. The first subject showed progressive disease on imaging at Day 14 following Ad+V and was subsequently shown to be pseudoprogression (PsP) with stable disease at Week 16 and 40, followed by partial response at Week 72 and Week 96 (FIG. 10). The first subject was administered all 15 doses of veledimex at 40 mg. The first subject also received a 10 mg cumulative dose of dexamethasone during veledimex dosing. The second subject was a 40 year old male with unifocal disease with one enhancing and one non-enhancing lesion and one prior line of therapy. The subject showed progressive disease at Day 28. This was subsequently shown to be pseudoprogression (PsP) with stable disease at Day 56 and partial response based on scans at Week 30 and Week 48 (FIG. 11). The second subject was administered 13 out of 15 doses of veledimex at 20 mg. The second subject also received a total of 8 mg dexamethasone during veledimex dosing.

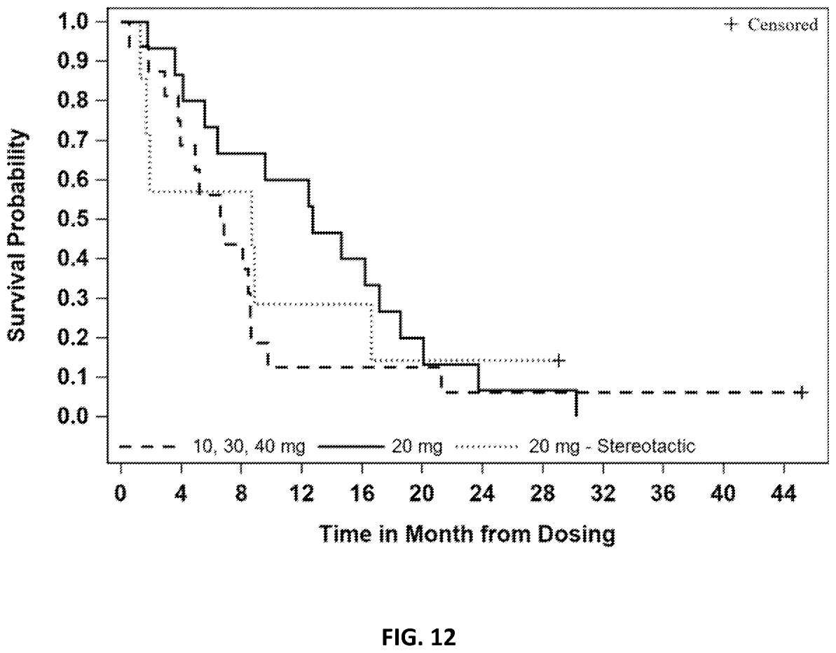

Survival in 10 mg, 30 mg and 40 mg Veledimex Subjects (Main Study). FIG. 12 and Table 14 show the survival of subjects from the main study. Data from unifocal and multifocal patients are combined.

TABLE 14
Survival

| | | | No. of | Median Survival | | Mean |
|---|---|---|---|---|---|---|
| | | No. of | Subjects | (95% CI) | | Follow-up |
| Group | Veledimex | Subjects | Alive | (mos) | | (mos) |
| Group 1 | 20 mg | 15 | 0 | 12.7 | (4.1, 17.1) | 13.1 |
| | 10 mg, 30 mg, 40 mg | 16 | 1 | 6.7 | (3.8, 8.6) | 9.1 |
| | 10 mg | 6 | 1 | 7.6 | (1.8, —) | 6.7 |
| | 30 mg | 4 | 0 | 3.4 | (0.5, —) | 3.1 |
| | 40 mg | 6 | 0 | 8.3 | (3.9, —) | 15.6 |
| Group 2 | 20 mg | 7 | 0 | 8.7 | (1.3, 16.6) | 9.7 |

### Survival in 10 mg, 30 mg and 40 mg Veledimex—Unifocal Patients (Main Study).

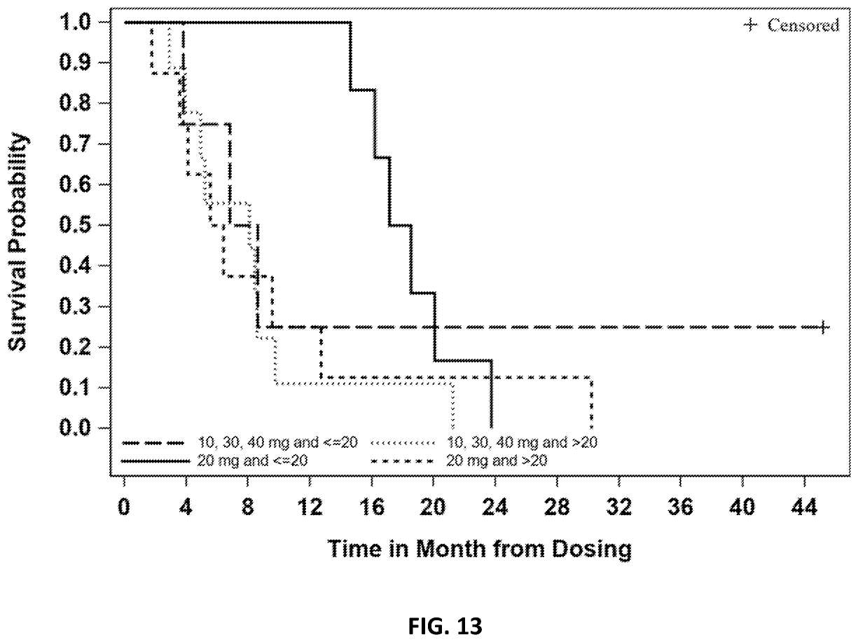

FIG. 13 and Table 15 show the survival of the unifocal subjects from the main study. Patients receiving low-dose concurrent steroids continued to show a trend towards longer survival.

TABLE 15
Survival

| Unifocal, | Cumulative | | No. of | Median Survival | | Mean |
|---|---|---|---|---|---|---|
| V Dose | Steroids | No. of | Subjects | (95% CI) | | Follow-up |
| Cohort | (Days 0-14) | Subjects | Alive | (mos) | | (mos) |
| 10, 30, | ≤20 mg | 4 | 1 | 7.7 | (3.8, —) | 16.1 |
| 40 mg | >20 mg | 9 | 0 | 8.1 | (2.9, 9.8) | 8.1 |
| 20 mg | ≤20 mg | 6 | 0 | 17.8 | (14.6, —) | 18.4 |
| | >20 mg | 8 | 0 | 6.0 | (1.8, 12.7) | 9.2 |

### Discussion and Conclusions.

The results of Main Study 10 mg, 30 mg and 40 mg V cohorts show efficacy for subjects with rGBM, especially for unifocal disease with low-dose steroids. These results are consistent with the 20 mg V cohort described in Example 3.

### Embodiments of the Invention Include

E1. A method of increasing the survival time of a subject having unifocal glioblastoma comprising
   a. intratumorally injecting an adenoviral vector, e.g., an Ad-RTS-hIL-12 viral vector, wherein the vector comprises:
      i. a first polynucleotide encoding a human IL-12 p40 polypeptide;
      ii. a second polynucleotide encoding a human IL-12 p35 polypeptide which is at least 85% identical to wild-type human IL-12 p35;
      iii. a third polynucleotide encoding a VP-16 transactivation domain-retinoic acid-X-receptor fusion protein (VP-16-RXR); and
      iv. a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and
   b. orally administering a diacylhydrazine ligand that activates the ligand-dependent transcription factor complex.
E2. The method of E1, wherein the increase in survival time is at least 1.3 fold higher than the anticipated survival times compared to historical controls.
E3. A method of treating unifocal glioblastoma in a subject comprising
   a. intratumorally injecting an adenoviral vector, e.g., an Ad-RTS-hIL-12 viral vector, wherein the vector comprises:
      i. a first polynucleotide encoding a human IL-12 p40 polypeptide;
      ii. a second polynucleotide encoding a human IL-12 p35 polypeptide which is at least 85% identical to wild-type human IL-12 p35;
      iii. a third polynucleotide encoding a VP-16 transactivation domain-retinoic acid-X-receptor fusion protein (VP-16-RXR); and
      iv. a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and
   b. orally administering a diacylhydrazine ligand that activates the ligand-dependent transcription factor complex.
E4. The method of any one of E1-E3, wherein, the first polynucleotide and the second polynucleotide is joined by a first linker.
E5. The method of any one of E1-E4, wherein, the third polynucleotide and the fourth polynucleotide is joined by a second linker.
E6. The method of E4 or E5, wherein the first linker and/or the second linker is an internal ribosome entry site (IRES) sequence.
E7. The method of E6, wherein the first linker and the second linker are different IRES sequences.
E8. The method of any one of E1-E7, wherein the vector is a replication-deficient adenoviral vector.
E9. The method of any one of E1-E8, wherein the subject has not received a steroid for at least 4 weeks prior to injection of the adenoviral vector, e.g., the Ad-RTS-hIL-12 viral vector.
E10. The method of any one of E1-E9, wherein the subject has not previously received bevacizumab.
E11. The method of any one of E1-E10, wherein an initial dose of the vector and an initial dose of the diacylhydrazine ligand are administered concurrently or sequentially.
E12. The method of any one of E1-E11, wherein an initial dose of the diacylhydrazine ligand is administered at a period of time prior to an initial dose of the vector.
E13. The method of any one of E1-E12, where the initial dose of the diacylhydrazine ligand is administered at about 1 to 5 hours prior to the administration of the vector.
E14. The method of any one of E11-E13, wherein one or more subsequent doses of the diacylhydrazine ligand are administered once daily after the administration of the initial dose of the diacylhydrazine ligand.
E15. The method of any one of E1-E14, wherein the subsequent daily doses of the diacylhydrazine ligand are administered for a period of time of about 3-28 days. E16. The method of E15, wherein the period of time is 14 days.
E17. The method of any one of E1-E16, further comprising administering to the subject a corticosteroid.
E18. The method of E18, wherein the corticosteroid is dexamethasone.
E19. The method of E17 or E18, wherein the cumulative dose of corticosteroid during the administration of diacylhydrazine ligand is less than or equal to about 20 mg.
E20. The method of any one of E17-E19, wherein an initial does of 10 mg of corticosteroid is administered on the same day as the initial dose of the diacylhydrazine ligand.
E21. The method of E17 or E18, wherein the cumulative dose of corticosteroid during the administration of diacylhydrazine ligand is greater than about 20 mg
E22. The method of any one of E1-E20, wherein the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp).
E23. The method of any one of E1-E22, wherein the vector is administered at a dose of about 2×10¹¹ vp.
E24. The method of any one of E1-E23, wherein the diacylhydrazine ligand is (R)—N′-(3,5-dimethylbenzoyl)-N′-(2,2-dimethylhexan-3-yl)-2-ethyl-3-methoxybenzohydrazide.
E25. The method of any one of E1-E24, wherein the diacylhydrazine ligand is administered at a unit daily dose of about 1 mg to about 120 mg.
E26. The method of any one of E1-E25, wherein the diacylhydrazine ligand is administered at unit daily dose of about 5, 10, 15, 20, 25, 30, 35, 40, 50, 60, 70, 80, 90, 100 or 120 mg.
E27. The method of any one of E1-E26, wherein the diacylhydrazine ligand is administered at a unit daily dose of about 5 mg.
E28. The method of any one of E1-E27, wherein the diacylhydrazine ligand is administered at a unit daily dose of about 10 mg.
E29. The method of any one of E1-E28, wherein the diacylhydrazine ligand is administered at a unit daily dose of about 15 mg.
E30. The method of any one of E1-E29, wherein the diacylhydrazine ligand is administered at a unit daily dose of about 20 mg.
E31. The method of any one of E1-E30, wherein the diacylhydrazine ligand is veledimex.
E32. A method of increasing the survival time of a subject having multifocal glioblastoma comprising
   a. intratumorally injecting an adenoviral vector, e.g., an Ad-RTS-hIL-12 viral vector, wherein the vector comprises:
      i. a first polynucleotide encoding a human IL-12 p40 polypeptide;
      ii. a second polynucleotide encoding a human IL-12 p35 polypeptide which is at least 85% identical to wild-type human IL-12 p35;
      iii. a third polynucleotide encoding a VP-16 transactivation domain-retinoic acid-X-receptor fusion protein (VP-16-RXR); and
      iv. a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and
   b. orally administering a diacylhydrazine ligand that activates the ligand-dependent transcription factor complex.
E33. The method of E32, wherein the increase in survival time is at least 1.3 fold higher than the anticipated survival times compared to historical controls.
E34. A method of treating multifocal glioblastoma in a subject comprising
   a. intratumorally injecting an adenoviral vector, e.g., an Ad-RTS-hIL-12 viral vector, wherein the vector comprises:
      i. a first polynucleotide encoding a human IL-12 p40 polypeptide;
      ii. a second polynucleotide encoding a human IL-12 p35 polypeptide which is at least 85% identical to wild-type human IL-12 p35;
      iii. a third polynucleotide encoding a VP-16 transactivation domain-retinoic acid-X-receptor fusion protein (VP-16-RXR); and
      iv. a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and
   b. orally administering a diacylhydrazine ligand that activates the ligand-dependent transcription factor complex.
E35. The method of any one of E32-E34, wherein, the first polynucleotide and the second polynucleotide is joined by a first linker.
E36. The method of any one of E32-E35, wherein, the third polynucleotide and the fourth polynucleotide is joined by a second linker.
E37. The method of E35 or E36, wherein the first linker and/or the second linker is an internal ribosome entry site (IRES) sequence.
E38. The method of E37, wherein the first linker and the second linker are different IRES sequences.
E39. The method of any one of E32-E38, wherein the vector is a replication-deficient adenoviral vector.
E40. The method of any one of E32-E39, wherein the subject has not received a steroid for at least 4 weeks prior to injection of the adenoviral vector, e.g., the Ad-RTS-hIL-12 viral vector.
E41. The method of any one of E32-E40, wherein the subject has not previously received bevacizumab.
E42. The method of any one of E32-E41, wherein an initial dose of the vector and an initial dose of the diacylhydrazine ligand are administered concurrently or sequentially.
E43. The method of any one of E32-E42, wherein an initial dose of the diacylhydrazine ligand is administered at a period of time prior to an initial dose of the vector.
E44. The method of any one of E32-E43, where the initial dose of the diacylhydrazine ligand is administered at about 1 to 5 hours prior to the administration of the vector.
E45. The method of any one of E32-E44, wherein one or more subsequent doses of the diacylhydrazine ligand are administered once daily after the administration of the initial dose of the diacylhydrazine ligand.
E46. The method of any one of E32-E45, wherein the subsequent daily doses of the diacylhydrazine ligand are administered for a period of time of about 3-28 days.
E47. The method of E46, wherein the period of time is 14 days.
E48. The method of any one of E32-E47, further comprising administering to the subject a corticosteroid.
E49. The method of E48, wherein the corticosteroid is dexamethasone.
E50. The method of E48 or E49, wherein the cumulative dose of corticosteroid during the administration of diacylhydrazine ligand is less than or equal to about 20 mg.
E51. The method of any one of E48-E50, wherein an initial does of 10 mg of corticosteroid is administered on the same day as the initial dose of the diacylhydrazine ligand.
E52. The method of E48 or E49, wherein the cumulative dose of corticosteroid during the administration of diacylhydrazine ligand is greater than about 20 mg.
E53. The method of any one of E32-E52, wherein the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹ or 1×10¹² or 2×10¹² viral particles (vp).
E54. The method of any one of E32-E53, wherein the vector is administered at a dose of about 2×10¹¹ vp.
E55. The method of any one of E32-E54, wherein the diacylhydrazine ligand is (R)—N′-(3,5-dimethylbenzoyl)-N′-(2,2-dimethylhexan-3-yl)-2-ethyl-3-methoxybenzohydrazide.
E56. The method of any one of E32-E55, wherein the diacylhydrazine ligand is administered at a unit daily dose of about 1 mg to about 120 mg.
E57. The method of any one of E32-E56, wherein the diacylhydrazine ligand is administered at unit daily dose of about 5, 10, 15, 20, 25, 30, 35, 40, 50, 60, 70, 80, 90, 100 or 120 mg.
E58. The method of any one of E32-E57, wherein the diacylhydrazine ligand is administered at a unit daily dose of about 5 mg.
E59. The method of any one of E32-E58, wherein the diacylhydrazine ligand is administered at a unit daily dose of about 10 mg.
E60. The method of any one of E32-E59, wherein the diacylhydrazine ligand is administered at a unit daily dose of about 15 mg.
E61. The method of any one of E32-E60, wherein the diacylhydrazine ligand is administered at a unit daily dose of about 20 mg.
E61. The method of any one of E32-E61, wherein the diacylhydrazine ligand is veledimex.

### Other Embodiments

While the invention has been described in conjunction with the detailed description thereof, the foregoing description is intended to illustrate and not limit the scope of the invention, which is defined by the scope of the appended claims. Other aspects, advantages and modifications are within the scope of the following claims.

1. A method of treating unifocal glioblastoma in a subject in need thereof comprising
a. intratumorally injecting into the subject an adenoviral vector, wherein the vector comprises:
i. a first polynucleotide encoding an IL-12 p40 polypeptide comprising an amino acid sequence at least 85% identical to wild-type human IL-12 p40 polypeptide;
ii. a second polynucleotide encoding an IL-12 p35 polypeptide comprising an amino acid sequence at least 85% identical to wild-type human IL-12 p35 polypeptide;
iii. a third polynucleotide encoding a VP-16 transactivation domain-retinoic acid-X-receptor fusion protein (VP-16-RXR); and
iv. a fourth polynucleotide encoding a Gal4 DNA binding domain and an ecdysone receptor (EcR) binding domain fusion protein (Gal4-EcR), wherein the VP-16-RXR fusion protein and the Gal4-EcR fusion protein form a ligand dependent transcription factor complex; and
b. orally administering to the subject a diacylhydrazine ligand that activates the ligand-dependent transcription factor complex,
thereby treating the unifocal glioblastoma in the subject.

2. (canceled)

3. The method of claim 1,
wherein the survival time of the subject is increased.

4. The method of claim 3, wherein the increase in survival time is at least 1.3 fold higher than survival times in subjects not administered the adenoviral vector.

5. The method of claim 1, wherein
(a) the IL-12 p40 polypeptide is a human IL-12 p40 peptide,
(b) the IL-12 p35 polypeptide is a human IL-12 p35 peptide, or
(c) both (a) and (b).

6. (canceled)

7. The method of claim 1, wherein
(a) the first polynucleotide and the second polynucleotide are joined by a first linker,
(b) the third polynucleotide and the fourth polynucleotide are joined by a second linker, or
(c) both (a) and (b).

8. (canceled)

9. The method of claim 7, wherein the first linker and/or the second linker is an internal ribosome entry site (IRES) sequence.

10. The method of claim 9, wherein the first linker and the second linker are different IRES sequences.

11. (canceled)

12. (canceled)

13. The method of claim 1, wherein the subject has not received a steroid for at least 4 weeks prior to injection of the adenoviral vector.

14. The method of claim 1, wherein the subject has not previously received bevacizumab.

15. The method of claim 1, wherein an initial dose of the vector and an initial dose of the diacylhydrazine ligand are administered concurrently.

16. The method of claim 1, wherein an initial dose of the vector and an initial dose of the diacylhydrazine ligand are administered sequentially.

17. The method of claim 16, wherein an initial dose of the diacylhydrazine ligand is administered at a period of time prior to an initial dose of the vector.

18. (canceled)

19. (canceled)

20. (canceled)

21. (canceled)

22. The method of claim 1, further comprising administering to the subject a corticosteroid.

23. The method of claim 22, wherein the corticosteroid is dexamethasone.

24. (canceled)

25. (canceled)

26. (canceled)

27. (canceled)

28. (canceled)

29. The method of claim 1, wherein the vector is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹, 1×10¹² or 2×10¹² viral particles (vp).

30. (canceled)

31. The method of claim 1, wherein the diacylhydrazine ligand is administered at a unit daily dose of about 1 mg to about 120 mg.

32. (canceled)

33. (canceled)

34. (canceled)

35. (canceled)

36. (canceled)

37. (canceled)

38. (canceled)

39. (canceled)

40. The method of claim 1, further comprising selecting the subject with unifocal glioblastoma before injecting the adenoviral vector or orally administering the diacylhydrazine ligand.

41. (canceled)

42. The method of claim 40,
wherein the diacylhydrazine ligand is veledimex, which is orally administered to the subject daily.

43. (canceled)

44. The method of claim 42,
wherein the subject is also administered dexamethasone at a cumulative dose of less than 20 mg for at least two weeks after veledimex is first administered,
thereby increasing the survival time of the subject.


SEQUENCE LISTING (4 sequences)

SEQ ID NO: 1 - protein, 328 aa, Artificial Sequence
  Human IL 12 p40 polypeptide
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 219 aa, Artificial Sequence
  Human IL-12 p35 polypeptide
MGPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRLRAVTIDRVMSYLNAS

SEQ ID NO: 3 - protein, 324 aa, Artificial Sequence
  VP16-RXR fusion protein
MGPKKKRKVAPPTDVSLGDELHLDGEDVAMAHADALDDFDLDMLGDGDSPGPGFTPHDSAPYGALDMADFEFEQMFTDALGIDEYGGEFEMPVDRILEAELAVEQKSDQGVEGPGGTGGSGSSPNDPVTNICQAADKQLFTLVEWAKRIPHFSSLPLDDQVILLRAGWNELLIASFSHRSIDVRDGILLATGLHVHRNSAHSAGVGAIFDRVLTELVSKMRDMRMDKTELGCLRAIILFNPEVRGLKSAQEVELLREKVYAALEEYTRTTHPDEPGRFAKLLLRLPSLRSIGLKCLEHLFFFRLIGDVPIDTFLMEMLESPSDS

SEQ ID NO: 4 - protein, 488 aa, Artificial Sequence
  GaI4-EcR fusion protein
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFPGIRPECVVPETQCAMKRKEKKAQKEKDKLPVSTTTVDDHMPPIMQCEPPPPEAARIHEVVPRFLSDKLLVTNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL